(19) United States
(12) Patent Application Publication
(10) Pub. No.: US 2024/0101492 A1
(43) Pub. Date: Mar. 28, 2024

(54) FUNGAL ENDOPHYTES FOR IMPROVED CROP YIELDS AND PROTECTION FROM PESTS

(71) Applicant: The Texas A&M University System, College Station, TX (US)

(72) Inventor: Gregory A. SWORD, College Station, TX (US)

(21) Appl. No.: 18/465,931

(22) Filed: Sep. 12, 2023

Related U.S. Application Data
(63) Continuation of application No. 15/853,057, filed on Dec. 22, 2017, now Pat. No. 11,807,586.
(60) Provisional application No. 62/567,113, filed on Oct. 2, 2017.
(60) Provisional application No. 62/546,959, filed on Aug. 17, 2017.
(60) Provisional application No. 62/438,966, filed on Dec. 23, 2016.

(51) Int. Cl. C05F 11/08; A01C 1/06; A01H 17/00; A01N 25/04; A01N 63/30

(52) U.S. Cl. CPC C05F 11/08; A01C 1/06; A01H 17/00; A01N 25/04; A01N 63/30

(57) ABSTRACT

Synthetic compositions comprising a plant element and at least one fungal endophyte are described. The fungal endophyte is capable of improving plant tolerance to biotic stress as compared to a reference plant element not further comprising the endophyte. Examples of biotic stress include the biotic stress caused by a nematode, an aphid, a fleahopper, a Lygus bug, a stink bug, a soy looper, a cabbage looper, or a fungus.

### CROSS REFERENCE TO RELATED APPLICATIONS

This application is a continuation of U.S. application Ser. No. 15/853,057, filed Dec. 22, 2017, now issued U.S. Pat. No. 11,807,586, which claims priority and benefits to Provisional Application No. 62/438,966, filed Dec. 23, 2016; Provisional Application No. 62/546,959, filed Aug. 17, 2017; and Provisional Application No. 62/567,113, filed Oct. 2, 2017, the disclosures of which are incorporated by reference in their entirety.

### SEQUENCE LISTING

The instant application contains a Sequence Listing with 115 sequences which has been submitted via Patent Center and is hereby incorporated by reference in its entirety. Said .xml copy, created on Sep. 8, 2023, is named 32610-56462-US and is 189,836 bytes in size.

### FIELD OF THE INVENTION

The present invention relates to fungal endophytes of agricultural crops for improving yield and/or for protection from pests.

### BACKGROUND OF THE INVENTION

Fungal endophytes are fungi that internally colonize plant tissues without causing evident damage or disease. Particular fungal endophytes, such as mycorrhiza, survive within various host plant tissues, often colonizing the intercellular spaces of host leaves, stems, flowers or roots. The symbiotic endophyte-host relationships can provide several fitness benefits to the host plant, such as enhancement of nutrition, and/or increased drought tolerance. Root-colonizing mycorrhizae survive on photosynthetic carbohydrates from the plant, and in return, aid in the solubilization and uptake of water and minerals to the host, which can lead to the promotion of seed germination and plant growth. Additionally, the association of a fungal endophyte with a host plant can provide tolerance to a variety of biotic and abiotic stresses. Host growth, fitness promotion and protection are thought to be achieved through multiple beneficial properties of the endophyte-host association. For instance, the endophytic organisms may produce growth-regulating substances to induce biomass production and alkaloids or other metabolites. Additionally, fungal endophytes may directly suppress or compete with disease-causing microbes, protecting the plant from potential pathogens.

### SUMMARY OF THE INVENTION

In some embodiments, the invention described herein provides a synthetic composition, comprising a plant element and at least one fungal endophyte selected from Table 3, wherein the fungal endophyte is capable of improving plant tolerance to biotic stress as compared to a reference plant element not further comprising the endophyte.

In some embodiments, the invention described herein provides a synthetic composition, comprising: a) a fungal endophyte comprising at least one endophyte from Table 3; and b) at least one carrier, wherein the fungal endophyte is in contact with the carrier; and wherein the fungal endophyte, when heterologously disposed to a plant element, is capable of improving plant tolerance to biotic stress as compared to a reference plant element not further comprising the endophyte. In some embodiments, the carrier comprises alginic acid, carrageenan, dextrin, dextran, Pelgel™, polyethelene glycol, polyvinyl pyrrolidone, methyl cellulose, polyvinyl alcohol, gelatin, or combinations thereof. In some embodiments, the synthetic composition further comprises water, a detergent, an insecticide, a fungicide, or combinations thereof. In some embodiments, the weight ratio between fungal endophyte and carrier is 1:1-10, 1:10-50, 1:50-100, 1:100-500, 1:500-1000, or 1:1000-5000. In some embodiments, the synthetic composition is a fluid or a powder. In some embodiments of any of the compositions described herein, the composition comprises at least 1, 2, 3, 4, 5, 6, 7, 8, 9, 10, 11, 12, 13, 14, 15, 16, 17, 18, 19, or 20 endophytes. In some embodiments of any of the compositions described herein, the fungal endophyte comprises fungal spores. In some embodiments, the fungal spores are present in about 10², 10³, 10⁴, 10⁵, 10⁶, 10⁷, 10⁸, or 10⁹ colony forming units per gram or spores per gram. In some embodiments of any of the compositions provided herein, the composition further comprises a plant element. In some embodiments, the plant element is a dicot. In some embodiments, the dicot is soybean. In some embodiments, the dicot is cotton. In some embodiments, the plant element is a seed. In some embodiments of any of the synthetic compositions provided herein, the fungal endophyte is heterologously disposed to a seed in a seed coating. In some embodiments, the plant element comprises leaf tissue. In some embodiments of any of the synthetic compositions provided herein, the fungal endophyte is heterologously disposed to a leaf in a foliar spray or powder. In some embodiments, the plant element comprises root tissue. In some embodiments of any of the synthetic compositions provided herein, the fungal endophyte is heterologously disposed to a root in a root drench or soil treatment.

In some embodiments of any of the synthetic compositions provided herein, the at least one fungal endophyte is selected from the group consisting of: Cladosporium, Alternaria, Bipolaris, Chaetomium, Verticillium, Preussia, Pleospora, Epicoccum, or combinations thereof. In some embodiments, the fungal endophyte comprises a nucleic acid sequence that is at least 97% identical to a nucleic acid sequence selected from the group consisting of SEQ ID NOs: SEQ ID NOs: 26-115.

In some embodiments, the invention described herein provides a synthetic composition comprising a fungal endophyte capable of improving plant tolerance to biotic stress, wherein the biotic stress is caused by a nematode, an aphid, a fleahopper, a Lygus bug, a stink bug, a soy looper, a cabbage looper, a fungus, or combinations thereof. In some embodiments, the biotic stress is caused by root knot nematode. In some embodiments, biotic stress is caused by reniform nematode. In some embodiments, biotic stress is caused by a Lepidoptera larvae. In some embodiments, the biotic stress is caused by a Lepidoptera larvae of the family Noctuidae. In some embodiments, the biotic stress is caused by Chrysodeixis includens. In some embodiments, the biotic stress is caused by Trichoplusia ni. In some embodiments, the biotic stress is caused by a Hemiptera insect. In some embodiments, the biotic stress is caused by Nezara viridula. In some embodiments, the biotic stress is caused by Lygus hesperus. In some embodiments, the biotic stress is caused by Aphis gossypii. In some embodiments, the biotic stress is caused by a fungi of the genus Rhizoctonia. In some embodiments, the biotic stress is caused by Rhizoctonia solani. In some embodiments, the biotic stress is caused by a fungi of the genus Fusarium. In some embodiments, the biotic stress is Fusarium virguliforme. In some embodiments, the biotic stress is caused by Fusarium oxysporum. In some embodiments, the biotic stress is caused by a plant pest or pathogen and improved plant tolerance is demonstrated by at least increased emergence, increased stand, increased survival, increased plant height, increased shoot biomass, increased root biomass, decreased disease score, increased leaf area, decreased pest abundance, decreased pest biomass, increased yield, improved vigor, or improved resistance to pathogenic bacteria, fungi or viruses. In some embodiments, the pest is of an order selected from the group consisting of: Lepidoptera, Hemiptera, or Tylenchida. In some embodiments, the pathogen is of a genus selected from the group consisting of: Fusarium or Rhizoctonia.

In some embodiments, the invention described herein provides a method of improving a plant phenotype, comprising inoculating plant elements with a formulation comprising a fungal endophyte heterologously disposed to the plant elements, wherein: a) the fungal endophyte is selected from Table 3; b) a phenotype is improved as compared to plant elements of reference plants not inoculated with the formulation; and c) the plant phenotype is selected from the group consisting of: increased disease resistance, increased pest resistance, increased herbivore resistance, increased resistance to a fungal pathogen, increased resistance to a bacterial pathogen, increased resistance to a viral pathogen, increased resistance to a nematode, increased insect resistance, increased leaf area in the presence of a biotic stressor, increased yield in the presence of a biotic stressor, or combinations thereof. In some embodiments, the plant phenotype is increased yield in the presence of a biotic stressor and the increase of yield is at least about 1%, 2%, 3%, 4%, 5%, 10%, 15%, 20%, or 25%. In some embodiments, the plant phenotype is leaf area is at least about 5%, 15%, 20%, or 25%.

In some embodiments, the invention described herein provides a method for reducing damage due to biotic stress, comprising inoculating plant elements with a formulation comprising a fungal endophyte heterologously disposed to the plant elements, wherein the fungal endophyte comprises a nucleic acid sequence having at least 97% identity to a nucleic acid sequence selected in Table 3, wherein damage due to biotic stress is reduced as compared to plant elements of reference plants not inoculated with the formulation. In some embodiments, the crop is cotton and the reduction of damage comprises reduced boll damage. In some embodiments, the reduction of boll damage comprises a decrease in the loss of bolls of about 1%, 2%, 3%, 4%, 5%, 10%, 15%, 20%, 30%, 40%, or 45%. In some embodiments, the reduction of damage comprises increased leaf area of about 5%, 10%, 15%, 20%, 30%, 40%, or 45%. In some embodiments, the reduction of damage improves yield as compared to reference plants not inoculated with the formulation.

In some embodiments, the invention described herein provides a method for treating biotic stress, comprising inoculating plant elements with a formulation comprising a fungal endophyte heterologously disposed to the plant elements, wherein the fungal endophyte comprises a nucleic acid sequence having at least 97% identity to a nucleic acid sequence selected in Table 3, wherein the fungal endophyte is capable of improving tolerance to biotic stress in the plants comprising or derived from the inoculated plant elements compared to plants comprising or derived from reference plant elements not inoculated with the formulation.

In some embodiments, the invention described herein provides a method for preventing pest infestation, comprising inoculating plant elements with a formulation comprising a fungal endophyte heterologously disposed to the plant elements, wherein the fungal endophyte is selected from Table 3, wherein pests are less abundant on the plants comprising or derived from the inoculated plant elements compared to plants comprising or derived from reference plant elements not inoculated with the formulation.

In some embodiments, the invention described herein provides a method for preventing pest infestation, comprising inoculating plant elements with a formulation comprising a fungal endophyte heterologously disposed to the plant elements, wherein the fungal endophyte is selected from Table 3, wherein pests are smaller on the plants comprising or derived from the inoculated plant elements compared to plants comprising or derived from reference plant elements not inoculated with the formulation.

In some embodiments of any of the methods described herein, the fungal endophyte is selected from the group consisting of: Cladosporium, Alternaria, Bipolaris, Chaetomium, Verticillium, Preussia, Pleospora, Epicoccum, or combinations thereof.

In some embodiments of any of the methods described herein, the fungal endophyte comprises a nucleic acid sequence that is at least 97% identical to a nucleic acid sequence selected from the group consisting of SEQ ID NOs: 26-115. In some embodiments of any of the methods described herein, the formulation comprises at least 1, 2, 3, 4, 5, 6, 7, 8, 9, 10, 11, 12, 13, 14, 15, 16, 17, 18, 19, or 20 endophytes. In some embodiments of any of the methods described herein, the plant element is a seed. In some embodiments of any of the methods described herein, the plant element is a dicot. In some embodiments of any of the methods described herein, the dicot is soybean. In some embodiments of any of the methods described herein, the dicot is cotton. In some embodiments of any of the methods described herein, the method further comprises sterilizing the seeds to remove microorganisms prior to combining the seeds with the endophyte composition.

In some embodiments of any of the methods described herein for treating or preventing biotic stress, reducing plant damage due to biotic stress, or improving a plant phenotype of a plant experiencing biotic stress, the biotic stress is caused by a nematode, an aphid, a fleahopper, a Lygus bug, a stink bug, a soy looper, a cabbage looper, a fungus, or combinations thereof. In some embodiments the biotic stress is caused by root knot nematode. In some embodiments the biotic stress is caused by reniform nematode. In some embodiments the biotic stress is caused by a Lepidoptera larvae. In some embodiments the biotic stress is caused by a Lepidoptera larvae of the family Noctuidae. In some embodiments the biotic stress is caused by Chrysodeixis includens. In some embodiments the biotic stress is caused by Trichoplusia ni. In some embodiments the biotic stress is caused by a Hemiptera insect. In some embodiments the biotic stress is caused by Nezara viridula. In some embodiments the biotic stress is caused by Lygus Hesperus. In some embodiments the biotic stress is caused by Aphis gossypii. In some embodiments the biotic stress is caused by a fungi of the genus Rhizoctonia. In some embodiments the biotic stress is caused by Rhizoctonia solani. In some embodiments the biotic stress is caused by a fungi of the genus Fusarium. In some embodiments the biotic stress is caused by Fusarium virguliforme. In some embodiments the biotic stress is caused by Fusarium oxysporum.

### BRIEF DESCRIPTION OF THE DRAWINGS

FIG. 1. Percentage change in mean number of root-knot nematode galls in endophyte treated cotton plants relative to the control treatment; plants were treated and grown as described in Example 7. Symbol on each bar indicates a significant difference in number of galls relative to the control treatment, * indicates p-value of <0.05.

FIG. 2. Percentage change in mean number of root-knot nematode galls in endophyte treated cotton plants relative to the control treatment; plants were treated and grown as described in Example 7. Symbol on each bar indicates a significant difference in number of galls relative to the control treatment, * indicates p-value of <0.05.

FIG. 3. Kaplan Meier survival curve of output of the Cox proportional hazards model for time to wilt of cotton seedlings treated with fungal endophyte strain MIC-07010. Day 0 represents sowing, plants were watered to saturation at day 7 and day 14, after which time water was withheld and wilting was scored on a daily basis; plants were treated and grown as described in Example 4.

FIG. 4. Kaplan Meier survival curve of output of the Cox proportional hazards model for time to wilt of cotton seedlings treated with fungal endophyte strain MIC-16066. Day 0 represents sowing, plants were watered to saturation at day 7 and day 14, after which time water was withheld and wilting was scored on a daily basis; plants were treated and grown as described in Example 4.

FIG. 5. Kaplan Meier survival curve of output of the Cox proportional hazards model for time to wilt of cotton seedlings treated with fungal endophyte strain MIC-20835. Day 0 represents sowing, plants were watered to saturation at day 7 and day 14, after which time water was withheld and wilting was scored on a daily basis; plants were treated and grown as described in Example 4.

FIG. 6. Kaplan Meier survival curve of output of the Cox proportional hazards model for time to wilt of cotton seedlings treated with fungal endophyte strain MIC-22947. Day 0 represents sowing, plants were watered to saturation at day 7 and day 14, after which time water was withheld and wilting was scored on a daily basis; plants were treated and grown as described in Example 4.

FIG. 7. Kaplan Meier survival curve of output of the Cox proportional hazards model for time to wilt of cotton seedlings treated with fungal endophyte strain MIC-23475. Day 0 represents sowing, plants were watered to saturation at day 7 and day 14, after which time water was withheld and wilting was scored on a daily basis; plants were treated and grown as described in Example 4.

FIG. 8. Kaplan Meier survival curve of output of the Cox proportional hazards model for time to wilt of cotton seedlings treated with fungal endophyte strain MIC-31246. Day 0 represents sowing, plants were watered to saturation at day 7 and day 14, after which time water was withheld and wilting was scored on a daily basis; plants were treated and grown as described in Example 4.

FIG. 9. Kaplan Meier survival curve of output of the Cox proportional hazards model for time to wilt of cotton seedlings treated with fungal endophyte strain MIC-31593. Day 0 represents sowing, plants were watered to saturation at day 7 and day 14, after which time water was withheld and wilting was scored on a daily basis; plants were treated and grown as described in Example 4.

FIG. 10. Kaplan Meier survival curve of output of the Cox proportional hazards model for time to wilt of cotton seedlings treated with fungal endophyte strain MIC-42067. Day 0 represents sowing, plants were watered to saturation at day 7 and day 14, after which time water was withheld and wilting was scored on a daily basis; plants were treated and grown as described in Example 4.

FIG. 11. Kaplan Meier survival curve of output of the Cox proportional hazards model for time to wilt of cotton seedlings treated with fungal endophyte strain MIC-50324. Day 0 represents sowing, plants were watered to saturation at day 7 and day 14, after which time water was withheld and wilting was scored on a daily basis; plants were treated and grown as described in Example 4.

FIG. 12. Kaplan Meier survival curve of output of the Cox proportional hazards model for time to wilt of cotton seedlings treated with fungal endophyte strain MIC-61920. Day 0 represents sowing, plants were watered to saturation at day 7 and day 14, after which time water was withheld and wilting was scored on a daily basis; plants were treated and grown as described in Example 4.

FIG. 13. Kaplan Meier survival curve of output of the Cox proportional hazards model for time to wilt of cotton seedlings treated with fungal endophyte strain MIC-62081. Day 0 represents sowing, plants were watered to saturation at day 7 and day 14, after which time water was withheld and wilting was scored on a daily basis; plants were treated and grown as described in Example 4.

FIG. 14. Kaplan Meier survival curve of output of the Cox proportional hazards model for time to wilt of cotton seedlings treated with fungal endophyte strain MIC-66827. Day 0 represents sowing, plants were watered to saturation at day 7 and day 14, after which time water was withheld and wilting was scored on a daily basis; plants were treated and grown as described in Example 4.

FIG. 15. Kaplan Meier survival curve of output of the Cox proportional hazards model for time to wilt of cotton seedlings treated with fungal endophyte strain MIC-72092. Day 0 represents sowing, plants were watered to saturation at day 7 and day 14, after which time water was withheld and wilting was scored on a daily basis; plants were treated and grown as described in Example 4.

FIG. 16. Kaplan Meier survival curve of output of the Cox proportional hazards model for time to wilt of cotton seedlings treated with fungal endophyte strain MIC-72531. Day 0 represents sowing, plants were watered to saturation at day 7 and day 14, after which time water was withheld and wilting was scored on a daily basis; plants were treated and grown as described in Example 4.

FIG. 17. Kaplan Meier survival curve of output of the Cox proportional hazards model for time to wilt of cotton seedlings treated with fungal endophyte strain MIC-90694. Day 0 represents sowing, plants were watered to saturation at day 7 and day 14, after which time water was withheld and wilting was scored on a daily basis; plants were treated and grown as described in Example 4.

FIG. 18. Kaplan Meier survival curve of output of the Cox proportional hazards model for time to wilt of cotton seedlings treated with fungal endophyte strain MIC-91347. Day 0 represents sowing, plants were watered to saturation at day 7 and day 14, after which time water was withheld and wilting was scored on a daily basis; plants were treated and grown as described in Example 4.

FIG. 19. Kaplan Meier survival curve of output of the Cox proportional hazards model for time to death of cotton seedlings treated with fungal endophyte strain MIC-07010. Day 0 represents sowing, plants were watered to saturation at day 7 and day 14, after which time water was withheld and death was scored on a daily basis; plants were treated and grown as described in Example 4.

FIG. 20. Kaplan Meier survival curve of output of the Cox proportional hazards model for time to death of cotton seedlings treated with fungal endophyte strain MIC-20835. Day 0 represents sowing, plants were watered to saturation at day 7 and day 14, after which time water was withheld and death was scored on a daily basis; plants were treated and grown as described in Example 4.

FIG. 21. Kaplan Meier survival curve of output of the Cox proportional hazards model for time to death of cotton seedlings treated with fungal endophyte strain MIC-26952. Day 0 represents sowing, plants were watered to saturation at day 7 and day 14, after which time water was withheld and death was scored on a daily basis; plants were treated and grown as described in Example 4.

FIG. 22. Kaplan Meier survival curve of output of the Cox proportional hazards model for time to death of cotton seedlings treated with fungal endophyte strain MIC-31593. Day 0 represents sowing, plants were watered to saturation at day 7 and day 14, after which time water was withheld and death was scored on a daily basis; plants were treated and grown as described in Example 4.

FIG. 23. Kaplan Meier survival curve of output of the Cox proportional hazards model for time to death of cotton seedlings treated with fungal endophyte strain MIC-39772. Day 0 represents sowing, plants were watered to saturation at day 7 and day 14, after which time water was withheld and death was scored on a daily basis; plants were treated and grown as described in Example 4.

FIG. 24. Kaplan Meier survival curve of output of the Cox proportional hazards model for time to death of cotton seedlings treated with fungal endophyte strain MIC-48747. Day 0 represents sowing, plants were watered to saturation at day 7 and day 14, after which time water was withheld and death was scored on a daily basis; plants were treated and grown as described in Example 4.

FIG. 25. Kaplan Meier survival curve of output of the Cox proportional hazards model for time to death of cotton seedlings treated with fungal endophyte strain MIC-59232. Day 0 represents sowing, plants were watered to saturation at day 7 and day 14, after which time water was withheld and death was scored on a daily basis; plants were treated and grown as described in Example 4.

FIG. 26. Kaplan Meier survival curve of output of the Cox proportional hazards model for time to death of cotton seedlings treated with fungal endophyte strain MIC-65047. Day 0 represents sowing, plants were watered to saturation at day 7 and day 14, after which time water was withheld and death was scored on a daily basis; plants were treated and grown as described in Example 4.

FIG. 27. Kaplan Meier survival curve of output of the Cox proportional hazards model for time to death of cotton seedlings treated with fungal endophyte strain MIC-72531. Day 0 represents sowing, plants were watered to saturation at day 7 and day 14, after which time water was withheld and death was scored on a daily basis; plants were treated and grown as described in Example 4.

FIG. 28. Kaplan Meier survival curve of output of the Cox proportional hazards model for time to death of cotton seedlings treated with fungal endophyte strain MIC-77047. Day 0 represents sowing, plants were watered to saturation at day 7 and day 14, after which time water was withheld and death was scored on a daily basis; plants were treated and grown as described in Example 4.

FIG. 29. Kaplan Meier survival curve of output of the Cox proportional hazards model for time to death of cotton seedlings treated with fungal endophyte strain MIC-85153. Day 0 represents sowing, plants were watered to saturation at day 7 and day 14, after which time water was withheld and death was scored on a daily basis; plants were treated and grown as described in Example 4.

FIG. 30. Kaplan Meier survival curve of output of the Cox proportional hazards model for time to death of cotton seedlings treated with fungal endophyte strain MIC-87929. Day 0 represents sowing, plants were watered to saturation at day 7 and day 14, after which time water was withheld and death was scored on a daily basis; plants were treated and grown as described in Example 4.

FIG. 31. Kaplan Meier survival curve of output of the Cox proportional hazards model for time to death of cotton seedlings treated with fungal endophyte strain MIC-96038. Day 0 represents sowing, plants were watered to saturation at day 7 and day 14, after which time water was withheld and death was scored on a daily basis; plants were treated and grown as described in Example 4.

FIG. 32. Hazard ratios derived from the Cox proportional hazards model for time to wilt of cotton seedlings treated with fungal endophyte strains. Hazard ratios are displayed in rank order from strains giving the most effective protection from the measured hazard (HR<1) to strains giving no protection from the measured hazard (HR >1).

FIG. 33. Hazard ratios derived from the Cox proportional hazards model for time to death of cotton seedlings treated with fungal endophyte strains. Hazard ratios are displayed in rank order from strains giving the most effective protection from the measured hazard (HR<1) to strains giving no protection from the measured hazard (HR >1).

FIG. 34. Larval survival (%) after 7 days of herbivory by cabbage looper larvae on a soy leaflet grown from seeds treated with fungal endophytes MIC-92234 (TAM00013), MIC-68178 (TAM00032), MIC-62081 (TAM00103), MIC-07010 (TAM00105), MIC-31593 (TAM00189), or MIC-12207 (TAM00296). GH6—This round received 3 larvae per leaflet and larvae were transferred to a fresh leaflet from the same plant after 5 days. n=15 plants per treatment.

FIG. 35. Larval survival (%) after 7 days of herbivory by cabbage looper larvae on a soy leaflet grown from seeds treated with fungal endophytes MIC-92234 (TAM00013), MIC-62081 (TAM00103), MIC-07010 (TAM00105), MIC-39772 (TAM00317), MIC-45943 (TAM00362), or MIC-44512 (TAM00560). GH7—This round received 3 larvae per leaflet and larvae were transferred to a fresh leaflet from the same plant after 5 days. n=15 plants per treatment.

FIG. 36. Average weight of surviving cabbage looper larvae (mg) after 7 days of herbivory on a soy leaflet grown from seeds treated with fungal endophytes MIC-92234 (TAM00013), MIC-62081 (TAM00103), MIC-07010 (TAM00105), MIC-39772 (TAM00317), MIC-45943 (TAM00362), or MIC-44512 (TAM00560). GH7—This round received 3 larvae per leaflet and larvae were transferred to a fresh leaflet from the same plant after 5 days. n=15 plants per treatment.

FIG. 37. Average weight of surviving cabbage looper larvae (mg) after 7 days of herbivory on a soy leaflet grown from seeds treated with fungal endophytes MIC-85555 (TAM00074), MIC-91557 (TAM00463), MIC-42665 (TAM00524), MIC-49739 (TAM00533), MIC-33414 (TAM00554), or MIC-22947 (TAM00559). GH10—This round received 3 larvae per leaflet and larvae were transferred to a fresh leaflet from the same plant after 5 days. n=15 plants per treatment.

FIG. 38. Leaf damage (%) as assessed via visual rating scale after 7 days of herbivory by cabbage looper larvae on a soy leaflet grown from seeds treated with fungal endophytes MIC-76091 (TAM00194), MIC-29439 (TAM00201), MIC-10602 (TAM00248), MIC-80602 (TAM00249), or MIC-51742 (TAM00251). GH3—This round received 3 larvae per leaflet and larvae were transferred to a fresh leaflet from the same plant after 5 days. n=15 plants per treatment. This batch of cabbage loopers had poor overall herbivory performance.

FIG. 39. Leaf damage (%) as assessed via visual rating scale after 7 days of herbivory by cabbage looper larvae on a soy leaflet grown from seeds treated with fungal endophytes MIC-92234 (TAM00013), MIC-62081 (TAM00103), MIC-07010 (TAM00105), MIC-39772 (TAM00317), MIC-45943 (TAM00362), or MIC-44512 (TAM00560). GH7—This round received 3 larvae per leaflet and larvae were transferred to a fresh leaflet from the same plant after 5 days. n=15 plants per treatment.

FIG. 40. Leaf damage (%) as assessed via visual rating scale after 7 days of herbivory by cabbage looper larvae on a soy leaflet grown from seeds treated with fungal endophytes MIC-90694 (TAM00046), MIC-77047 (TAM00100), MIC-63740 (TAM00504), MIC-17815 (TAM00518), MIC-16714 (TAM00531), or MIC-16066 (TAM00536). GH8—This round received 3 larvae per leaflet and larvae were transferred to a fresh leaflet from the same plant after 5 days. n=15 plants per treatment.

FIG. 41. Leaf damage (%) as assessed via visual rating scale after 7 days of herbivory by cabbage looper larvae on a soy leaflet grown from seeds treated with fungal endophytes MIC-85555 (TAM00074), MIC-91557 (TAM00463), MIC-42665 (TAM00524), MIC-49739 (TAM00533), MIC-33414 (TAM00554), or MIC-22947 (TAM00559). GH10—This round received 3 larvae per leaflet and larvae were transferred to a fresh leaflet from the same plant after 5 days. n=15 plants per treatment.

FIG. 42. Leaf area consumed (cm\hat{ }2) as calculated from starting leaf area and percent leaf damage assessed via visual rating scale after 7 days of herbivory by cabbage looper larvae on a soy leaflet grown from seeds treated with fungal endophytes MIC-76091 (TAM00194), MIC-29439 (TAM00201), MIC-10602 (TAM00248), MIC-80602 (TAM00249), or MIC-51742 (TAM00251). GH3—This round received 3 larvae per leaflet and larvae were transferred to a fresh leaflet from the same plant after 5 days. n=15 plants per treatment. This batch of cabbage loopers had poor overall herbivory performance.

FIG. 43. Leaf area consumed (cm\hat{ }2) as calculated from starting leaf area and percent leaf damage assessed via visual rating scale after 7 days of herbivory by cabbage looper larvae on a soy leaflet grown from seeds treated with fungal endophytes MIC-92234 (TAM00013), MIC-62081 (TAM00103), MIC-07010 (TAM00105), MIC-39772 (TAM00317), MIC-45943 (TAM00362), or MIC-44512 (TAM00560). GH7—This round received 3 larvae per leaflet and larvae were transferred to a fresh leaflet from the same plant after 5 days. n=15 plants per treatment.

FIG. 44. Leaf area consumed (cm\hat{ }2) as calculated from starting leaf area and percent leaf damage assessed via visual rating scale after 7 days of herbivory by cabbage looper larvae on a soy leaflet grown from seeds treated with fungal endophytes MIC-90694 (TAM00046), MIC-77047 (TAM00100), MIC-63740 (TAM00504), MIC-17815 (TAM00518), MIC-16714 (TAM00531), or MIC-16066 (TAM00536). GH8—This round received 3 larvae per leaflet and larvae were transferred to a fresh leaflet from the same plant after 5 days. n=15 plants per treatment.

FIG. 45. Leaf area consumed (cm\hat{ }2) as calculated from starting leaf area and percent leaf damage assessed via visual rating scale after 7 days of herbivory by cabbage looper larvae on a soy leaflet grown from seeds treated with fungal endophytes MIC-85555 (TAM00074), MIC-91557 (TAM00463), MIC-42665 (TAM00524), MIC-49739 (TAM00533), MIC-33414 (TAM00554), or MIC-22947 (TAM00559). GH10—This round received 3 larvae per leaflet and larvae were transferred to a fresh leaflet from the same plant after 5 days. n=15 plants per treatment.

FIG. 46. Leaf area consumed (cm\hat{ }2) as assessed normalized by average larval weight (mg) after 7 days of herbivory by cabbage looper larvae on a soy leaflet grown from seeds treated with fungal endophytes MIC-50989 (TAM00490), MIC-26952 (TAM00494), MIC-90504 (TAM00497), or MIC-50414 (TAM00534). GH2—This round received 3 larvae per leaflet and larvae were transferred to a fresh leaflet from the same plant after 5 days. n=15 plants per treatment.

FIG. 47. Leaf area consumed (cm\hat{ }2) as assessed normalized by average larval weight (mg) after 7 days of herbivory by cabbage looper larvae on a soy leaflet grown from seeds treated with fungal endophytes MIC-76091 (TAM00194), MIC-29439 (TAM00201), MIC-10602 (TAM00248), MIC-80602 (TAM00249), or MIC-51742 (TAM00251). GH3—This round received 3 larvae per leaflet and larvae were transferred to a fresh leaflet from the same plant after 5 days. n=15 plants per treatment. This batch of cabbage loopers had poor overall herbivory performance.

FIG. 48. Leaf area consumed (cm\hat{ }2) as assessed normalized by average larval weight (mg) after 7 days of herbivory by cabbage looper larvae on a soy leaflet grown from seeds treated with fungal endophytes MIC-92234 (TAM00013), MIC-68178 (TAM00032), MIC-62081 (TAM00103), MIC-07010 (TAM00105), MIC-31593 (TAM00189), or MIC-12207 (TAM00296). GH6—This round received 3 larvae per leaflet and larvae were transferred to a fresh leaflet from the same plant after 5 days. n=15 plants per treatment.

FIG. 49. Leaf area consumed (cm\hat{ }2) as assessed normalized by average larval weight (mg) after 7 days of herbivory by cabbage looper larvae on a soy leaflet grown from seeds treated with fungal endophytes MIC-92234 (TAM00013), MIC-62081 (TAM00103), MIC-07010 (TAM00105), MIC-39772 (TAM00317), MIC-45943 (TAM00362), or MIC-44512 (TAM00560). GH7—This round received 3 larvae per leaflet and larvae were transferred to a fresh leaflet from the same plant after 5 days. n=15 plants per treatment.

FIG. 50. Leaf area consumed (cm\hat{ }2) as assessed normalized by average larval weight (mg) after 7 days of herbivory by cabbage looper larvae on a soy leaflet grown from seeds treated with fungal endophytes MIC-90694 (TAM00046), MIC-77047 (TAM00100), MIC-63740 (TAM00504), MIC-17815 (TAM00518), MIC-16714 (TAM00531), or MIC-16066 (TAM00536). GH8—This round received 3 larvae per leaflet and larvae were transferred to a fresh leaflet from the same plant after 5 days. n=15 plants per treatment.

FIG. 51. Leaf area consumed (cm\hat{ }2) as assessed normalized by average larval weight (mg) after 7 days of herbivory by cabbage looper larvae on a soy leaflet grown from seeds treated with fungal endophytes MIC-20571 (TAM00160), MIC-12927 (TAM00193), MIC-39233 (TAM00323), MIC-45943 (TAM00362), MIC-77538 (TAM00439), MIC-32308 (TAM00473), or MIC-31246 (TAM00501). GH9—This round received 3 larvae per leaflet and larvae were transferred to a fresh leaflet from the same plant after 5 days. n=15 plants per treatment.

FIG. 52. Leaf area consumed (cm\hat{ }2) as assessed normalized by average larval weight (mg) after 7 days of herbivory by cabbage looper larvae on a soy leaflet grown from seeds treated with fungal endophytes MIC-85555 (TAM00074), MIC-91557 (TAM00463), MIC-42665 (TAM00524), MIC-49739 (TAM00533), MIC-33414 (TAM00554), or MIC-22947 (TAM00559). GH10—This round received 3 larvae per leaflet and larvae were transferred to a fresh leaflet from the same plant after 5 days. n=15 plants per treatment.

FIG. 53. Relationship between cabbage looper larval weight and leaf area consumed, data from all rounds of the assay are shown.

FIG. 54. Larval survival (%) after 7 days of herbivory by soy looper larvae on a soy leaflet grown from seeds treated with fungal endophytes MIC-62081 (TAM00103), MIC-68178 (TAM00032), MIC-62081 (TAM00103), MIC-07010 (TAM00105), MIC-31593 (TAM00189), or MIC-12207 (TAM00296). GH6—This round received 3 larvae per leaflet and larvae were transferred to a fresh leaflet from the same plant after 5 days. n=15 plants per treatment.

FIG. 55. Larval survival (%) after 7 days of herbivory by soy looper larvae on a soy leaflet grown from seeds treated with fungal endophytes MIC-92234 (TAM00013), MIC-62081 (TAM00103), MIC-07010 (TAM00105), MIC-39772 (TAM00317), MIC-45943 (TAM00362), or MIC-44512 (TAM00560). GH7—This round received 3 larvae per leaflet and larvae were transferred to a fresh leaflet from the same plant after 5 days. n=15 plants per treatment.

FIG. 56. Average weight of surviving larvae (mg) after 7 days of herbivory by soy looper larvae on a soy leaflet grown from seeds treated with fungal endophytes MIC-76091 (TAM00194), MIC-29439 (TAM00201), MIC-10602 (TAM00248), MIC-80602 (TAM00249), or MIC-51742 (TAM00251). GH3—This round received 3 larvae per leaflet and larvae were transferred to a fresh leaflet from the same plant after 5 days. n=15 plants per treatment.

FIG. 57. Average weight of surviving larvae (mg) after 7 days of herbivory by soy looper larvae on a soy leaflet grown from seeds treated with fungal endophytes MIC-92234 (TAM00013), MIC-62081 (TAM00103), MIC-07010 (TAM00105), MIC-39772 (TAM00317), MIC-45943 (TAM00362), or MIC-44512 (TAM00560). GH7—This round received 3 larvae per leaflet and larvae were transferred to a fresh leaflet from the same plant after 5 days. n=15 plants per treatment.

FIG. 58. Average weight of surviving larvae (mg) after 7 days of herbivory by soy looper larvae on a soy leaflet grown from seeds treated with fungal endophytes MIC-85555 (TAM00074), MIC-91557 (TAM00463), MIC-42665 (TAM00524), MIC-49739 (TAM00533), MIC-33414 (TAM00554), or MIC-22947 (TAM00559). GH10—This round received 3 larvae per leaflet and larvae were transferred to a fresh leaflet from the same plant after 5 days. n=15 plants per treatment.

FIG. 59. Leaf damage (%) as assessed via visual rating scale after 7 days of by soy looper larvae herbivory on a soy leaflet grown from seeds treated with fungal endophytes MIC-76091 (TAM00194), MIC-29439 (TAM00201), MIC-10602 (TAM00248), MIC-80602 (TAM00249), or MIC-51742 (TAM00251). GH3—This round received 3 larvae per leaflet and larvae were transferred to a fresh leaflet from the same plant after 5 days. n=15 plants per treatment.

FIG. 60. Leaf damage (%) as assessed via visual rating scale after 7 days of by soy looper larvae herbivory on a soy leaflet grown from seeds treated with fungal endophytes MIC-92234 (TAM00013), MIC-68178 (TAM00032), MIC-62081 (TAM00103), MIC-07010 (TAM00105), MIC-31593 (TAM00189), or MIC-12207 (TAM00296). GH6—This round received 3 larvae per leaflet and larvae were transferred to a fresh leaflet from the same plant after 5 days. n=15 plants per treatment.

FIG. 61. Leaf damage (%) as assessed via visual rating scale after 7 days of by soy looper larvae herbivory on a soy leaflet grown from seeds treated with fungal endophytes MIC-92234 (TAM00013), MIC-62081 (TAM00103), MIC-07010 (TAM00105), MIC-39772 (TAM00317), MIC-45943 (TAM00362), or MIC-44512 (TAM00560). GH7—This round received 3 larvae per leaflet and larvae were transferred to a fresh leaflet from the same plant after 5 days. n=15 plants per treatment.

FIG. 62. Leaf damage (%) as assessed via visual rating scale after 7 days of by soy looper larvae herbivory on a soy leaflet grown from seeds treated with fungal endophytes MIC-90694 (TAM00046), MIC-77047 (TAM00100), MIC-63740 (TAM00504), MIC-17815 (TAM00518), MIC-16714 (TAM00531), or MIC-16066 (TAM00536). GH8—This round received 3 larvae per leaflet and larvae were transferred to a fresh leaflet from the same plant after 5 days. n=15 plants per treatment.

FIG. 63. Leaf damage (%) as assessed via visual rating scale after 7 days of by soy looper larvae herbivory on a soy leaflet grown from seeds treated with fungal endophytes MIC-85555 (TAM00074), MIC-91557 (TAM00463), MIC-42665 (TAM00524), MIC-49739 (TAM00533), MIC-33414 (TAM00554), or MIC-22947 (TAM00559). GH10—This round received 3 larvae per leaflet and larvae were transferred to a fresh leaflet from the same plant after 5 days. n=15 plants per treatment.

FIG. 64. Leaf area consumed (cm\hat{ }2) as calculated from starting leaf area and percent leaf damage assessed via visual rating scale after 7 days of herbivory by soy looper larvae on a soy leaflet grown from seeds treated with fungal endophytes MIC-92234 (TAM00013), MIC-62081 (TAM00103), MIC-07010 (TAM00105), MIC-39772 (TAM00317), MIC-45943 (TAM00362), or MIC-44512 (TAM00560). GH7—This round received 3 larvae per leaflet and larvae were transferred to a fresh leaflet from the same plant after 5 days. n=15 plants per treatment.

FIG. 65. Leaf area consumed (cm\hat{ }2) as calculated from starting leaf area and percent leaf damage assessed via visual rating scale after 7 days of herbivory by soy looper larvae on a soy leaflet grown from seeds treated with fungal endophytes MIC-90694 (TAM00046), MIC-77047 (TAM00100), MIC-63740 (TAM00504), MIC-17815 (TAM00518), MIC-16714 (TAM00531), or MIC-16066 (TAM00536). GH8—This round received 3 larvae per leaflet and larvae were transferred to a fresh leaflet from the same plant after 5 days. n=15 plants per treatment.

FIG. 66. Leaf area consumed (cm\hat{ }2) as calculated from starting leaf area and percent leaf damage assessed via visual rating scale after 7 days of herbivory by soy looper larvae on a soy leaflet grown from seeds treated with fungal endophytes MIC-85555 (TAM00074), MIC-91557 (TAM00463), MIC-42665 (TAM00524), MIC-49739 (TAM00533), MIC-33414 (TAM00554), or MIC-22947 (TAM00559). GH10—This round received 3 larvae per leaflet and larvae were transferred to a fresh leaflet from the same plant after 5 days. n=15 plants per treatment.

FIG. 67. Leaf area consumed (cm\hat{ }2) as assessed normalized by average larval weight (mg) after 7 days of herbivory by soy looper larvae on a soy leaflet grown from seeds treated with fungal endophytes MIC-50989 (TAM00490), MIC-26952 (TAM00494), MIC-90504 (TAM00497), or MIC-50414 (TAM00534). GH2—This round received 3 larvae per leaflet and larvae were transferred to a fresh leaflet from the same plant after 5 days. n=15 plants per treatment.

FIG. 68. Leaf area consumed (cm\hat{ }2) as assessed normalized by average larval weight (mg) after 7 days of herbivory by soy looper larvae on a soy leaflet grown from seeds treated with fungal endophytes MIC-76091 (TAM00194), MIC-29439 (TAM00201), MIC-10602 (TAM00248), MIC-80602 (TAM00249), or MIC-51742 (TAM00251). GH3—This round received 3 larvae per leaflet and larvae were transferred to a fresh leaflet from the same plant after 5 days. n=15 plants per treatment.

FIG. 69. Leaf area consumed (cm\hat{ }2) as assessed normalized by average larval weight (mg) after 7 days of herbivory by soy looper larvae on a soy leaflet grown from seeds treated with fungal endophytes MIC-92234 (TAM00013), MIC-68178 (TAM00032), MIC-62081 (TAM00103), MIC-07010 (TAM00105), MIC-31593 (TAM00189), or MIC-12207 (TAM00296). GH6—This round received 3 larvae per leaflet and larvae were transferred to a fresh leaflet from the same plant after 5 days. n=15 plants per treatment.

FIG. 70. Leaf area consumed (cm\hat{ }2) as assessed normalized by average larval weight (mg) after 7 days of herbivory by soy looper larvae on a soy leaflet grown from seeds treated with fungal endophytes MIC-90694 (TAM00046), MIC-77047 (TAM00100), MIC-63740 (TAM00504), MIC-17815 (TAM00518), MIC-16714 (TAM00531), or MIC-16066 (TAM00536). GH8—This round received 3 larvae per leaflet and larvae were transferred to a fresh leaflet from the same plant after 5 days. n=15 plants per treatment.

FIG. 71. Leaf area consumed (cm\hat{ }2) as assessed normalized by average larval weight (mg) after 7 days of herbivory by soy looper larvae on a soy leaflet grown from seeds treated with fungal endophytes MIC-20571 (TAM00160), MIC-12927 (TAM00193), MIC-39233 (TAM00323), MIC-45943 (TAM00362), MIC-77538 (TAM00439), MIC-32308 (TAM00473), or MIC-31246 (TAM00501). GH9—This round received 3 larvae per leaflet and larvae were transferred to a fresh leaflet from the same plant after 5 days. n=15 plants per treatment.

FIG. 72. Leaf area consumed (cm\hat{ }2) as assessed normalized by average larval weight (mg) after 7 days of herbivory by soy looper larvae on a soy leaflet grown from seeds treated with fungal endophytes MIC-85555 (TAM00074), MIC-91557 (TAM00463), MIC-42665 (TAM00524), MIC-49739 (TAM00533), MIC-33414 (TAM00554), or MIC-22947 (TAM00559). GH10—This round received 3 larvae per leaflet and larvae were transferred to a fresh leaflet from the same plant after 5 days. n=15 plants per treatment.

FIG. 73. Relationship between soy looper larval weight and leaf area consumed, data from all rounds of the assay are shown.

FIG. 74. Exemplary photographs showing disease rating at a 0-3 scale (3 denotes strong disease symptoms) using the split-root scoring system at 45 days post planting are shown. FIG. 74A shows an exemplary photo of a healthy root showing no disease symptoms which would receive a “0” rating. FIG. 74B shows an exemplary photo of a root showing mild disease symptoms which would receive a “1” rating. FIG. 74C shows an exemplary photo of a root showing mild disease symptoms which would receive a “2” rating. FIG. 74D shows an exemplary photo of a root showing strong disease symptoms which would receive a “3” rating.

FIG. 75. This figure shows the mean distance moved by Southern Green Stink Bugs (Nezara viridula) when in closed containers with cotton bolls collected from endophyte treated and reference (“control”) plants, as captured during the 6 hour observation period in video behavior assays described in Example 11. Stink bugs in the presence of cotton bolls from plants treated with MIC-92234 (TAM00013) and MIC-68178 (TAM00032), on average, moved shorter distances during the observation period than did stink bugs in the presence of untreated reference plants.

FIG. 76. This figure shows the mean distance of Southern Green Stink Bugs (Nezara viridula) from the boll when in closed containers with cotton bolls collected from endophyte treated and reference (“control”) plants, as captured during the 6 hour observation period in video behavior assays described in Example 11. Stink bugs in the presence of cotton bolls from plants treated with MIC-92234 (TAM00013) and MIC-90694 (TAM00046), on average, stayed farther away from the boll than stink bugs in the presence of untreated reference plants.

FIG. 77. This figure shows the mean frequency of Southern Green Stink Bugs (Nezara viridula) coming in contact with or in direct proximity (within 0.5 cm) to the boll when in closed containers with cotton bolls collected from endophyte treated and reference (“control”) plants, as captured during the 6 hour observation period in video behavior assays described in Example 11. Stink bugs in the presence of cotton bolls from plants treated with MIC-92234 (TAM00013), MIC-68178 (TAM00032) and MIC-90694 (TAM00046), on average, had less frequent contact with the boll zone than stink bugs in the presence of untreated reference plants.

FIG. 78. This figure shows the mean time until first contact with the boll by Southern Green Stink Bugs (Nezara viridula) when in closed containers with cotton bolls collected from endophyte treated and reference (“control”) plants, as captured during the 6 hour observation period in video behavior assays described in Example 11. Stink bugs in the presence of cotton bolls from plants treated with MIC-92234 (TAM00013) and MIC-90694 (TAM00046), on average, took more time to make first contact with the boll than stink bugs in the presence of untreated reference plants. Each 1 increment on the y-axis represents 20 seconds.

FIG. 79. This figure shows the mean percent of the 6 hour observation period that Southern Green Stink Bugs (Nezara viridula) spent in contact with or in direct proximity of the boll when in closed containers with cotton bolls collected from endophyte treated and reference (“control”) plants, as captured during the video behavior assays described in Example 11. Stink bugs in the presence of cotton bolls from plants treated with MIC-92234 (TAM00013), on average, spent less than 20% of the observation period on or in the direct proximity of the boll. In contrast, stink bugs in the presence of untreated reference plants spent, on average, more than 35% of the observation period on or in the direct proximity of the boll. Insects in direct proximity of the boll are within the region directly surrounding the boll referred to as the “boll zone”; the boll zone is depicted in FIG. 80 B.

FIG. 80. This figure shows a schematic petri dish “arena” as used in the video behavior assays described in Example 11. A cotton boll (labeled C) is visible at the center of the plate. The region surrounding the boll is represented by the shaded region labeled B; this region is referred to as the boll zone. The boll zone is the region in direct proximity to the cotton boll. The outer area with the label A shows the region of the plate which is not in direct proximity to the cotton boll. A Southern Green Stink Bug (Nezara viridula) is depicted at the lower right edge of the arena and is labeled D.

FIG. 81. This figure shows an exemplary photo of 20 petri plate arenas as used in the video behavior assays described in Example 11. A cotton boll is visible at the enter of each arena. The other dark mass in each arena is a Southern Green Stink Bug (Nezara viridula). The lines within each arena represent the output of the video tracking software and are a visualization of the path over which the insect in that arena has traveled over the observation period.

FIG. 82 exemplifies the total number of aphids on each plant 7 days after infestation, for cottons grown from seeds treated with fungal endophytes TAM00452 (MIC-26235), TAM00514 (MIC-39051), TAM00317 (MIC-39772), TAM00129 (MIC-39830), MIC-39959 (TAM00333), TAM00560 (MIC-44512), TAM00089 (MIC-67271), TAM00340 (MIC-87502), MIC-96038 (TAM00505) or treatment controls; plants were treated and grown as described in Example 10.

FIG. 83 exemplifies the total number of aphids on each plant 7 days after infestation, for cottons grown from seeds treated with fungal endophytes TAM00248 (MIC-10602), TAM00565 (MIC-20835), (MIC-23475), TAM00501 (MIC-31246), TAM00474 (MIC-34220), SYM02486 (MIC-42067), TAM00110 (MIC-66827), TAM00179 (MIC-86713), TAM00340 (MIC-87502), or treatment controls; plants were treated and grown as described in Example 10.

FIG. 84 exemplifies the total number of aphids on each plant 7 days after infestation, for cottons grown from seeds treated with fungal endophytes TAM00193 (MIC-12927), TAM00424 (MIC-21610), TAM00416 (MIC-31674), TAM00190 (MIC-42406), TAM00413 (MIC-50324), TAM00508 (MIC-72092), TAM00517 (MIC-72531), TAM00415 (MIC-87929), TAM00169 (MIC-91347) or treatment controls; plants were treated and grown as described in Example 10.

FIG. 85 exemplifies the total number of aphids on each plant 7 days after infestation, for cottons grown from seeds treated with fungal endophytes TAM00304 (MIC-17794), TAM00201 (MIC-29439), TAM00489 (MIC-38632), TAM00529 (MIC-61920), TAM00057 (MIC-65885), TAM00512 (MIC-67609), TAM00032 (MIC-68178), TAM00497 (MIC-90504), TAM00526 (MIC-95013) or treatment controls; plants were treated and grown as described in Example 10.

FIG. 86 exemplifies the total number of alates on each plant 7 days after infestation, for cottons grown from seeds treated with fungal endophytes TAM00193 (MIC-12927), TAM00424 (MIC-21610), TAM00416 (MIC-31674), TAM00190 (MIC-42406), TAM00413 (MIC-50324), TAM00508 (MIC-72092), TAM00517 (MIC-72531), TAM00415 (MIC-87929), TAM00169 (MIC-91347) or treatment controls; plants were treated and grown as described in Example 10.

### DETAILED DESCRIPTION

### Definitions

In the description and tables herein, a number of terms are used. In order to provide a clear and consistent understanding of the specification and claims, the following definitions are provided. Unless otherwise noted, terms are to be understood according to conventional usage by those of ordinary skill in the relevant art.

When a term is provided in the singular, the inventors also contemplate aspects of the invention described by the plural of that term. The singular form “a,” “an,” and “the” include plural references unless the context clearly dictates otherwise. For example, the term “a cell” includes one or more cells, including mixtures thereof.

The term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and agriculturally acceptable carriers. “Consisting of” shall mean excluding more than trace elements of other ingredients and substantial method steps for applying the compositions of this invention. Embodiments defined by each of these transition terms are within the scope of this invention.

Biological control: the term “biological control” and its abbreviated form “biocontrol,” as used herein, is defined as control of a pest, pathogen, or insect or any other undesirable organism by the use of at least one endophyte.

As used herein, an “agricultural seed” is a seed used to grow plants in agriculture (an “agricultural plant”). The seed may be of a monocot or dicot plant, and is planted for the production of an agricultural product, for example grain, food, fiber, etc. As used herein, an agricultural seed is a seed that is prepared for planting, for example, in farms for growing. Agricultural seeds are distinguished from commodity seeds in that the former is not used to generate products, for example commodity plant products.

A “plant element” is intended to generically reference either a whole plant or a plant component, including but not limited to plant tissues, parts, and cell types. A plant element is preferably one of the following: whole plant, seedling, meristematic tissue, ground tissue, vascular tissue, dermal tissue, seed, leaf, root, shoot, stem, flower, fruit, stolon, bulb, tuber, corm, keikis, shoot, bud.

As used herein, a “commodity plant product” refers to any composition or product that is comprised of material derived from a plant, seed, plant cell, or plant part of the present invention. Commodity plant products may be sold to consumers and can be viable or nonviable. Nonviable commodity products include but are not limited to nonviable seeds and grains; processed seeds, seed parts, and plant parts; dehydrated plant tissue, frozen plant tissue, and processed plant tissue; seeds and plant parts processed for animal feed for terrestrial and/or aquatic animal consumption, oil, meal, flour, flakes, bran, fiber, and any other food for human or animal consumption; and biomasses and fuel products. Any such commodity plant product that is derived from the plants of the present invention may contain at least a detectable amount of the specific and unique DNA corresponding to the endophytes described herein. Any standard method of detection for polynucleotide molecules may be used, including methods of detection disclosed herein.

As used herein, the phrase “agronomically elite plants” refers to a genotype or cultivar with a phenotype adapted for commercial cultivation. Traits comprised by an agronomically elite plant may include biomass, carbohydrate, and/or seed yield; biotic or abiotic stress resistance, including drought resistance, insect resistance, fungus resistance, virus resistance, bacteria resistance, cold tolerance, and salt tolerance; improved standability, enhanced nutrient use efficiency, and reduced lignin content.

In certain embodiments, cotton agronomically elite plants include, for example, known cotton varieties AM 1550 B2RF, NG 1511 B2RF, NG 1511 B2RF, FM 1845LLB2, FM 1944GLB2, FM 1740B2F, PHY 499 WRF, PHY 375 WRF, PHY 367 WRF, PHY 339 WRF, PHY 575 WRF, DP 1252 B2RF, DP 1050 B2RF, DP 1137 B2RF, DP 1048 B2RF, and/or DP 1137 B2RF.

As used herein, the phrase “culture filtrate” refers to broth or media obtained from cultures inoculated with a strain of fungi and allowed to grow. The media is typically filtered to remove any suspended cells, leaving the nutrients, hormones, or other chemicals.

As used herein, the term “endophyte” refers to an organism capable of living within a plant or plant tissue. An endophyte may comprise a fungal organism that may confer an increase in yield, biomass, resistance, or fitness in its host plant. Fungal endophytes may occupy the intracellular or extracellular spaces of plant tissue, including the leaves, stems, flowers, or roots.

The phrase “pest resistance” refers to inhibiting or reducing attack from pests. Pest resistance provides at least some increase in pest resistance over that which is already possessed by the plant. In some embodiments, a pest is of an order selected from the group consisting of: Lepidoptera, Hemiptera, or Tylenchida.

As used herein, the term “genotypes” refers to the genetic constitution of a cell or organism.

As used herein, the term “phenotype” refers to the detectable characteristics of a cell or organism, which characteristics are either the direct or indirect manifestation of gene expression.

As used herein, the phrase “host plant” refers to any plant that an endophytic fungi colonizes. In certain embodiments, the host plant comprises progeny of colonized plant.

As used herein, the phrase “increased yield” refers to an increase in biomass or seed weight, seed or fruit size, seed number per plant, seed number per unit area, bushels per acre, tons per acre, kilo per hectare, carbohydrate yield, or cotton yield. Such increased yield is relative to a plant or crop that has not been inoculated with the endophyte. In certain embodiments, the increase yield is relative to other commonly used pest treatments or other methods of addressing the biotic or abiotic stress.

As used herein, the phrase “biomass” means the total mass or weight (fresh or dry), at a given time, of a plant tissue, plant tissues, an entire plant, or population of plants, usually given as weight per unit area. The term may also refer to all the plants or species in the community (community biomass).

As used herein, an “agriculturally acceptable” excipient or carrier is one that is suitable for use in agriculture without undue adverse side effects to the plants, the environment, or to humans or animals who consume the resulting agricultural products derived therefrom commensurate with a reasonable benefit/risk ratio.

In some embodiments, a treatment is applied to a plant or plant element by heterologously disposing the treatment to the plant or plant element. A treatment is “heterologously disposed” when mechanically or manually applied, artificially inoculated or disposed onto or into a plant element, seedling, plant or onto or into a plant growth medium or onto or into a treatment formulation so that the treatment exists on or in the plant element, seedling, plant, plant growth medium, or formulation in a manner not found in nature prior to the application of the treatment, e.g., said combination which is not found in nature in that plant variety, at that time in development, in that tissue, in that abundance, or in that growth condition (for example drought).

In some embodiments, a treatment is applied mechanically or manually or artificially inoculated to a plant element in a seed treatment, root wash, seedling soak, foliar application, soil inocula, in-furrow application, sidedress application, soil pre-treatment, wound inoculation, drip tape irrigation, vector-mediation via a pollinator, injection, osmopriming, hydroponics, aquaponics, aeroponics, and combinations thereof. Application to the plant may be achieved, for example, as a powder for surface deposition onto plant leaves, as a spray to the whole plant or selected plant element, as part of a drip to the soil or the roots, or as a coating onto the plant element prior to or after planting. Such examples are meant to be illustrative and not limiting to the scope of the invention.

A “synthetic composition” comprises one or more endophytes combined by human endeavor with a heterologously disposed plant element or a treatment formulation, said combination which is not found in nature. In some embodiments, the term “synthetic composition” means one or more plant elements or formulation components combined by human endeavor with an isolated, purified endophyte composition. In some embodiments, said purified endophyte composition is mechanically or manually applied, artificially inoculated or disposed on a plant element in a manner that is not found on or in the plant element before application of the purified endophyte composition, e.g., said combination or association which is not found in nature. In some embodiments, “synthetic composition” is used to refer to a treatment formulation comprising an isolated, purified population of endophytes heterologously disposed to a plant element. In some embodiments, “synthetic composition” refers to a purified population of endophytes in a treatment formulation comprising additional compositions with which said endophytes are not found in nature.

A “treatment formulation” refers to a mixture of chemicals that facilitate the stability, storage, and/or application of the endophyte composition(s). Treatment formulations may comprise any one or more agents such as: surfactant, a buffer, a tackifier, a microbial stabilizer, a fungicide, an anticomplex agent, an herbicide, a nematicide, an insecticide, a plant growth regulator, a rodenticide, a desiccant, a nutrient, an excipient, a wetting agent, a salt.

In some embodiments, an “agriculturally compatible carrier” or “carrier” can be used to formulate an agricultural formulation or other composition that includes a purified endophyte preparation. As used herein an “agriculturally compatible carrier” refers to any material, that can be added to a plant element without causing or having an adverse effect on the plant element (e.g., reducing seed germination) or the plant that grows from the plant element, or the like. In some embodiments, the weight ratio between fungal endophyte and a carrier is 1:1-10, 1:10-50, 1:50-100, 1:100-500, 1:500-1000, or 1:1000-5000. As used herein, a carrier may be a “sticker”. A sticker is a compound to enhance binding of spores to the seed surface, non-limiting examples of such compounds are alginic acid, carrageenan, dextrin, dextran, Pelgel™, polyethelene glycol, polyvinyl pyrrolidone, methyl cellulose, polyvinyl alcohol, or gelatin. In some embodiments, a composition comprising a carrier further comprises water, a detergent, an insecticide, a fungicide, or combinations thereof.

The present invention contemplates the use of “isolated” microbe. As used herein, an isolated microbe is a microbe that is isolated from its native environment, and carries with it an inference that the isolation was carried out by the hand of man. An isolated microbe is one that has been separated from at least some of the components with which it was previously associated (whether in nature or in an experimental setting) or occurs at a higher concentration, viability, or other functional aspect than occurring in its native environment. Therefore, an “isolated” microbe is partially or completely separated from any other substance(s) as it is found in nature or as it is cultured, propagated, stored or subsisted in naturally or non-naturally occurring environments. Specific examples of isolated microbes include partially pure microbes, substantially pure microbes and microbes cultured in a medium that is non-naturally occurring.

As used herein, a microbe is considered to be “native” to a plant or a portion of the plant, and is said to be “natively” present in the plant or a portion of plant, if that plant or portion of the plant contains the microbe, for example, in the absence of any contacting with the microbe preparation, or contains the microbe at much lower concentrations than the contacting with the microbe preparation would provide.

Some of the methods described herein allow the colonization of plant seeds by microbes. As used herein, a microbe is said to “colonize” a plant or seed when it can exist in a symbiotic or non-detrimental relationship with the plant in the plant environment, for example on, in close proximity to or inside a plant, including the seed. The terms “percent colonization”, “percentage of colonization”, and derivations thereof are used interchangeably and as used herein refer to the percent of individual plants sampled within each experimental treatment that exhibited evidence of positive colonization. Similarly, the term “colonization frequency” and derivations thereof, as used herein, refer to the number of individual plants sampled within each experimental treatment that exhibited evidence of positive colonization. Methods of determining positive colonization are well known in the art and include, for example: sequencing, microscopy and culture based methods.

A “population” of plants, as used herein, refers to a plurality of plants that were either grown from the seeds treated with the endophytes as described herein, or are progeny of a plant or group of plants that were subjected to the inoculation methods. The plants within a population are typically of the same species, and/or typically share a common genetic derivation.

A “reference plant”, “reference plant element”, “reference agricultural plant” or “reference seed” a similarly situated plant or seed of the same species, strain, or cultivar to which a treatment, formulation, composition or endophyte preparation as described herein is not administered/contacted. A reference plant, therefore, is identical to the treated plant except for the presence of the active ingredient to be tested and can serve as a control for detecting the effects of the treatment conferred to the plant. A plurality of reference plants may be referred to as a “reference population”.

### Endophytes

Endophytic fungi are ubiquitous in nature, infecting virtually all plants in both natural and agronomic ecosystems. Plants commonly harbor a diversity of fungi living within their tissues as asymptomatic endophytes that can provide protection from a range of biotic and abiotic stressors. The present disclosure describes certain fungal endophytes that can be pathogens, parasites or antagonists to plant pathogens, insects, and nematode pests, thereby providing health and performance benefits to crop plants. The symbiotic endophyte-host relationships can provide several general health and fitness benefits to the host plant, such as enhancement of nutrition, increased drought tolerance and/or chemical defense from potential herbivores and often enhanced biomass production. Root-colonizing mycorrhizae survive on photosynthetic carbohydrates from the plant, and in return, aid in the solubilization and uptake of water and minerals to the host, which can lead to the promotion of seed germination and plant growth. Additionally, the association of a fungal endophyte with a host plant often provides protection from pathogens or tolerance to a variety of biotic and abiotic stresses, such as insect infestation, grazing, water or nutrient deficiency, heat stress, salt or aluminum toxicity, and freezing temperatures. Host growth and fitness promotion and protection are thought to be achieved through multiple beneficial properties of the endophyte-host association.

These fungal endophytes provided in Table 3 were originally collected as fungal endophytes of cotton. These endophytic fungi can be inoculated to live within cotton using either seed, soil or foliar applications and exhibited surprisingly beneficial effects by providing protection from pest infestation. Pests can be nematode and/or insect pests.

Described is the application of beneficial fungi to establish endophytically within crop plants to improve plant performance and yield while conferring protection against insect and nematode pests. In this regard, the present invention overcomes the limitations of the prior art such as the susceptibility of the fungi to degradation by UV light, desiccation or heat after exposure to the environment following application as an inundative soil or foliar biopesticide. Inoculation and endophytic establishment of the fungi within the plant protects the fungi from UV light, desiccation, and unfavorable temperatures, while harboring the fungi in the very plant tissues they are intended to protect. Introducing fungi to live endophytically within plants requires no genetic modification of the plant or microorganisms, and the fungi themselves can be a source for natural products. In various embodiments, the fungal inoculant can be formulated and applied, for example, as treatment of seeds, in furrow applications, before or during planting, or as foliar application after plant germination, and after inoculation, the fungal endophytes provide season-long protective effects and higher crop yields (approximately 25% higher). In certain embodiments, the increase of yield is about 1%, 2%, 3%, 4%, 5%, 10%, 15%, 20%, 30%, 40%, 45%, 50%, or greater than 50% relative to a crop to which no endophyte composition has been applied. In further embodiments, the increase of yield is the result of reduction of loss that comprises reduction of loss due to insect infestation or drought and the loss is less than 50%, 40%, 30%, 20%, 10%, 5%, or 5% relative to a crop to which no endophyte composition has been applied. In certain embodiments, the crop is cotton and the reduction of loss comprises reduced boll damage.

The fungal endophyte may be present in intercellular spaces within plant tissue, such as the root. Its presence may also occur or may also be maintained within a plant or plant population by means of grafting or other inoculation methods such as treating seeds, plants or parts thereof with endophyte mycelia, or endophyte spores. In certain embodiments, the plant, part of the plant, roots, seed, or leaves are sterilized to remove microorganisms before applying the endophyte. In particular embodiments, seeds are sterilized to remove microorganisms prior to combining the seeds with the endophyte compositions herein described. In certain aspects, the ability of the seed to germinate is not affected by the sterilization. In particular embodiments, the plant surface is sterilized to remove microorganisms prior to applying a foliar treatment with the endophyte compositions herein described.

The invention also provides methods for detecting the presence of the fungal endophyte of the present invention within a host plant. This may be accomplished, for instance, by isolation of total DNA from tissues of a potential plant-endophyte combination, followed by PCR, or alternatively, Southern blotting, western blotting, or other methods known in the art, to detect the presence of specific nucleic or amino acid sequences associated with the presence of a fungal endophyte strain of the present invention. Alternatively, biochemical methods such as ELISA, HPLC, TLC, or fungal metabolite assays may be utilized to determine the presence of an endophyte strain of the present invention in a given sample of crop tissue. Additionally, methods for identification may include microscopic analysis, such as root staining, or culturing methods, such as grow out tests or other methods known in the art (Deshmukh et al. 2006). In particular embodiments, the roots of a potential plant-endophyte combination may be stained with fungal specific stains, such as WGA-Alexa 488, and microscopically assayed to determine fungal root associates.

Metabolomic differences between the plants can be detected using methods known in the art. For example, a biological sample (whole tissue, exudate, phloem sap, xylem sap, root exudate, etc.) from the endophyte-associated and reference agricultural plants can be analyzed essentially as described in Fiehn et al., (2000) Nature Biotechnol., 18, 1157-1161, or Roessner et al., (2001) Plant Cell, 13, 11-29. Such metabolomic methods can be used to detect differences in levels in hormones, nutrients, secondary metabolites, root exudates, phloem sap content, xylem sap content, heavy metal content, and the like.

In another embodiment, the present invention contemplates methods of coating the seed of a plant with a plurality of endophytes, as well as seed compositions comprising a plurality of endophytes on and/or in the seed. In some embodiments, a seed coating comprises at least 1, 2, 3, 4, 5, 6, 7, 8, 9, 10, 11, 12, 13, 14, 15, 16, 17, 18, 19, or 20 endophytes. The methods according to this embodiment can be performed in a manner similar to those described herein for single endophyte coating. In one example, multiple endophytes can be prepared in a single preparation that is coated onto the seed. The endophytes can be from a common origin (i.e., a same plant). Alternatively, the endophytes can be from different plants.

Where multiple endophytes are coated onto the seed, any or all of the endophytes may be capable of conferring a beneficial trait onto the host plant. In some cases, all of the endophytes are capable of conferring a beneficial trait onto the host plant. The trait conferred by each of the endophytes may be the same (e.g., both improve the host plant's tolerance to a particular biotic stress), or may be distinct (e.g., one improves the host plant's tolerance to drought, while another improves phosphate utilization). In other cases, the conferred trait may be the result of interactions between the endophytes.

In certain embodiments, the agronomic qualities may be selected from the group consisting of: increased disease resistance, increased pest resistance, increased herbivore resistance, increased resistance to a fungal pathogen, increased resistance to a bacterial pathogen, increased resistance to a viral pathogen, increased resistance to a nematode, increased insect resistance, increased leaf area in the presence of a biotic stressor, increased yield in the presence of a biotic stressor, or combinations thereof, each of these qualities being rated in comparison to otherwise identical plants grown under the same conditions, and differing only with respect to the presence or absence of a fungal endophyte. The synthetic combinations and methods of the present invention may be applied to respond to actual or anticipated stresses.

Plant-parasitic nematodes are distributed worldwide and parasitize almost all higher plants. They feed and reproduce on living plant cells in roots, and induce formation of giant cells and galls, which leads to disrupted plant water and nutrient uptake that can damage crops and reduce yields. External symptoms due to nematode infection include various degrees of stunting and wilting. In some embodiments, secondary infection by other pathogens may lead to decay of nematode-infected tissues. Non-limiting examples of nematode pests include root knot nematode (Meloidogyne incognita) and Reniform nematode (Rotylenchulus reniformis).

Current nematode control practices include chemical and cultural control with some use of host plant resistance. Increasing awareness of environmental and human safety has greatly reduced the amount of chemical usage and number of new nematicides approved for use. Studies using nematophagous microbes as biological control agents for nematode management have received more attention as the withdrawal of several nematicides (e.g. methyl bromide, dichloropropene, aldicarb and phenamiphos) from market increases the need for new nematode control strategies. An alternative to the application of fungal biological control agents to the soil for nematode control is the manipulation of the presence of fungal endophytes within the plant.

The present disclosure provides, in one embodiment, fungal endophytes selected from those in Table 3 that negatively affect the reproduction of plant parasitic nematodes attacking roots below ground, including knot nematodes (Meloidogyne incognita) and reniform nematodes (Rotylenchulus reniformis). Increased resistance to root knot nematodes was demonstrated in cotton, for example, employing Chaetomium globosum as an endophyte in greenhouse trials. In some embodiments, improved plant performance and yields in endophyte treated versus control plants can be observed in field trials. In some embodiments, the endophyte treatment is applied to a seed. In some embodiments, the endophyte treatment is a foliar treatment. In some embodiments, the endophyte treatment is a root drench. In some embodiments, an endophyte treatment comprises at least 1, 2, 3, 4, 5, 6, 7, 8, 9, 10, 11, 12, 13, 14, 15, 16, 17, 18, 19, or 20 endophytes. In some embodiments, an endophyte treatment comprises culture filtrate.

The present disclosure provides, in one embodiment, fungal endophytes selected from those in Table 3 that negatively affect the abundance and size of plant pests of the Order Lepidoptera also known as “chewing” insects. The larval stages of several Lepidopteran insects can cause serious to agricultural crops, particularly dicots including cotton and soybean. Defoliation due to excessive herbivory reduces the photosynthetic capacity of crops and is associated with reduced fruit and seed yield. Non-limiting examples of such of Lepidopteran insects include soybean looper (Chrysodeixis includens or Pseudoplusia includens) and cabbage looper (Trichoplusia ni). Increased resistance to soybean and cabbage looper in endophyte treated plants can be demonstrated by increased yield, improved vigor, improved resistance to fungal pathogens, or increased leaf area as compared to a reference plant element not further comprising the endophyte. In some embodiments, improved plant performance and yields in endophyte treated versus control plants can be observed in field trials. In some embodiments, fungal endophytes capable of improving plant performance under chewing insect pressure are selected from the genera Cladosporium, Alternaria, Bipolaris, Chaetomium, Verticillium, Preussia, Pleospora, or Epicoccum. In some embodiments, fungal endophytes capable of improving plant performance under chewing insect pressure comprises a nucleic acid sequence that is at least 97% identical to a nucleic acid sequence selected from the group consisting of SEQ ID NOs: 26-115.

The present disclosure provides, in one embodiment, fungal endophytes selected from those in Table 3 that negatively affect the affinity of piercing-sucking insects for endophyte treated plant tissue or plants derived from treated seeds or treated plants. Many piercing-sucking insects are of the Order Hemiptera and feed on plants. Non-limiting example of a piercing-sucking insects include aphids, thrips, fleahoppers, Lygus bugs (members of the genus Lygus), and stink bugs including the brown marmorated stink bug (Halyomorpha halys) and southern green stink bugs (Nezara viridula). In some embodiments, treatment of a plant with one or more fungal endophytes affects piercing-sucking insect behavior by decreasing the amount of time insects spend on plants or plant elements including their reproductive tissue (for example, cotton bolls), decreasing the number of times an insect approaches a plant or plant element, decreasing the number of insects that contact a plant or plant element, or increasing the amount of time before an insect approaches a plant or plant element, compared to a reference plant or plant element not further comprising the endophyte. In some embodiments, reducing the affinity of a piercing-sucking insect for a plant or plant element reduces the damage to the plant or plant element by insect feeding or infection by pathogenic bacteria, fungi or viruses. In some embodiments, reduced damage by piercing-sucking insects can be demonstrated by increased yield, improved vigor, or improved resistance pathogenic bacteria, fungi or viruses. In some embodiments, improved vigor includes a reduction in yellowing, wilting, deformation or stunting of plant tissue as compared to a reference plant tissue. In some embodiments, fungal endophytes capable of improving plant performance under piercing-sucking insect pressure are selected from the genera Cladosporium, Alternaria, Bipolaris, Chaetomium, Verticillium, Preussia, Pleospora, or Epicoccum. In some embodiments, fungal endophytes capable of improving plant performance under piercing-sucking insect pressure comprises a nucleic acid sequence that is at least 97% identical to a nucleic acid sequence selected from the group consisting of SEQ ID NOs: 26-115.

In some embodiments, the methods of preventing or treating a pest infestation provide a benefit to the treated plant by reducing the abundant of pests on the plants. In some embodiments, the reduced the abundant of pests on the plants is measured by counting the number of immature pests or pest eggs on the endophyte treated plant tissue. In some embodiments, the reduction in pest abundance is due to decreased survival of pests feeding on endophyte treated plants. In some embodiments, the reduction in pest abundance is due to the decreased attractiveness of endophyte treated plants to pests. In some embodiments, the decreased attractiveness of endophyte treated plants to pests is measured by, as non-limiting examples: decreased movement of pests, increased time of pests to move toward endophyte treated plants, decreased frequency of visits by the pest to the plant, or decreased time spent on or feeding on endophyte treated plants. In some embodiments, the methods of preventing or treating a pest infestation provide a benefit to the treated plant by reducing the biomass of feeding pests. In some embodiments, the pests on endophyte treated plants are visibly smaller. In some embodiments, the pests on endophyte treated plants are smaller as determined by measuring the pests' biomass.

A method for preventing pest infestation, comprising inoculating plant elements with a formulation comprising a fungal endophyte heterologously disposed to the plant elements, wherein the fungal endophyte is selected from Table 3, wherein pests are smaller on the plants comprising or derived from the inoculated plant elements compared to plants comprising or derived from reference plant elements not inoculated with the formulation.

In some embodiments, treatment or prevention of a biotic stress condition in a plant caused by a nematode, insect, fungi or bacteria with a fungal endophyte may reduce the frequency or rate of application of chemical nematocides, insecticides, fungicides or bactericides by 1%, 2%, 3%, 4%, 5%, 10%, 15%, 20%, 30%, 40%, 50%, 60%, 70%, 80%, 90% or 100%.

As used herein, a nucleic acid has “homology” or is “homologous” to a second nucleic acid if the nucleic acid sequence has a similar sequence to the second nucleic acid sequence. The terms “identity”, “percent identity”, “percent sequence identity” or “identical” in the context of nucleic acid sequences refer to the nucleotides in the two sequences that are the same when aligned for maximum correspondence. There are different algorithms known in the art that can be used to measure nucleotide sequence identity. Nucleotide sequence identity can be measured by a local or global alignment, preferably implementing an optimal local or optimal global alignment algorithm. For example, a global alignment may be generated using an implementation of the Needleman-Wunsch algorithm (Needleman, S. B. & Wunsch, C. D. (1970) Journal of Molecular Biology. 48(3):443-53). For example, a local alignment may be generated using an implementation of the Smith-Waterman algorithm (Smith T. F & Waterman, M. S. (1981) Journal of Molecular Biology. 147(1):195-197). Optimal global alignments using the Needleman-Wunsch algorithm and optimal local alignments using the Smith-Waterman algorithm are implemented in USEARCH, for example USEARCH version v8.1.1756_i86osx32.

A gap is a region of an alignment wherein a sequence does not align to a position in the other sequence of the alignment. In global alignments, terminal gaps are discarded before identity is calculated. For both local and global alignments, internal gaps are counted as differences. A terminal gap is a region beginning at the end of a sequence in an alignment wherein the nucleotide in the terminal position of that sequence does not correspond to a nucleotide position in the other sequence of the alignment and extending for all contiguous positions in that sequence wherein the nucleotides of that sequence do not correspond to a nucleotide position in the other sequence of the alignment. An internal gap is a gap in an alignment which is flanked on the 3′ and 5′ end by positions wherein the aligned sequences are identical.

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 76%, 80%, 85%, or at least about 90%, or at least about 95%, 96%, at least 97%, 98%, 99% or 100% of the positions of the alignment, wherein the region of alignment is at least about 50%, 60%, 70%, 75%, 85%, or at least about 90%, or at least about 95%, 96%, 97%, 98%, 99% or 100% of the length of the query sequence. In a preferred embodiment, inference of homology from a sequence alignment is make where the region of alignment is at least 85% of the length of the query sequence. In a preferred embodiment, the region of alignment contains at least 100 positions inclusive of any internal gaps. In some embodiments, the region of alignment comprises at least 100 nucleotides of the query sequence. In some embodiments, the region of alignment comprises at least 200 nucleotides of the query sequence. In some embodiments, the region of alignment comprises at least 300 nucleotides of the query sequence. In some embodiments, the region of alignment comprises at least 400 nucleotides of the query sequence. In some embodiments, the region of alignment comprises at least 500 nucleotides of the query sequence. In some embodiments, the query sequence is selected from the SEQ ID Nos in Table 3.

Historical taxonomic classification of fungi has been according to morphological presentation. Beginning in the mid-1800's, it was recognized that some fungi have a pleomorphic life cycle, and that different nomenclature designations were being used for different forms of the same fungus. With the development of genomic sequencing, it became evident that taxonomic classification based on molecular phylogenetics did not align with morphological-based nomenclature (Shenoy B D, Jeewon R, Hyde K D. Impact of DNA sequence-data on the taxonomy of anamorphic fungi. Fungal Diversity 26(10) 1-54. 2007). Systematics experts have not aligned on common nomenclature for all fungi, nor are all existing databases and information resources inclusive of updated taxonomies. As such, many fungi referenced herein may be described by their anamorph form but it is understood that based on identical genomic sequencing, any pleomorphic state of that fungus may be considered to be the same organism. In some cases, fungal genera have been reassigned due to various reasons, and it is understood that such nomenclature reassignments are within the scope of any claimed taxonomic classification.

For example, the genus Bipolaris and the genus Curvularia are closely related, but separate anamorphs, although the genus Cochliobolus has been described as the teleomorph for both. It is understood that the genus Acremonium is also reported in the literature as genus Sarocladium as well as genus Tilachilidium (Summerbell R. C., C. Gueidan, H-J. Schroers, G. S. de Hoog, M. Starink, Y. Arocha Rosete, J. Guarro and J. A. Scott. Acremonium phylogenetic overview and revision of Gliomastix, Sarocladium, and Trichothecium. Studies in Mycology 68: 139-162. 2011). Further, it is understood that the genus Cladosporium is an anamorph of the teleomorph genus Davidiella (Bensch K, Braun U, Groenewald J Z, Crous P W. The genus Cladosporium. Stud Mycol. 2012 Jun. 15; 72(1): 1-401), and is understood to describe the same organism. Stemphylium herbarum has been reported in the literature as the anamorph of Pleospora herbarum (Simmons, E. G. (1985). Perfect states of Stemphylium II.—Sydowia 38: 284-293). Additionally the literature has suggested that Verticillium nigrescens be reassigned to the genus Gibellulopsis (Zane, Rasoul & Gams, Walter & Starink-Willemse, Mieke & Summerbell, Richard. (2007). Gibellulopsis, a suitable genus for Verticillium nigrescens, and Musicillium, a new genus for V theobromae. Nova Hedwigia. 85. 463-489. 10.1127/0029-5035/2007/0085-0463).

### EXAMPLES

### Example 1: Isolation of Endophytic Fungi

Endophytic fungi were obtained from cotton plants as described (Ek-Ramos et al. 2013, PLoS ONE 8(6): e66049. doi:10.1371/journal.pone.0066049), except Beauveria bassiana which was cultured from a commercially obtained strain (available from Botanigard, BioWorks). Persons of ordinary skill in the art can obtain endophytes suitable for performing the various embodiments of the present invention by performing the procedures described therein. In short, plant samples were rinsed in tap water and surface sterilized by immersion in 70% ethanol for 5 min, 10% bleach solution for 3 min, and rinsed twice with autoclaved distilled water. Samples were blotted dry using autoclaved paper towels. Five individual surface sterilized leaves, squares and bolls (N=15 total samples) were randomly selected and imprinted onto fresh potato dextrose agar (PDA) and V8 media as a way to monitor surface sterilization efficiency. For endophyte isolation, leaves were cut in small fragments of approximately 1 square cm. Squares and bolls were cut in six pieces. Any fiber present was removed and cut into six smaller pieces. Leaf fragments were placed upside down on PDA and V8 medium plates in triplicate. Each plate contained 3 leaf fragments for a total of 9 fragments assayed per plant. For squares collected early in the season, 3 slices per square were plated on PDA and V8 media as with the leaf fragments. Because of similarity in size and location within a plant, when collected later in the season, squares and bolls from a given plant were plated together on petri dishes containing two square slices, two boll slices and two pieces of fiber. Antibiotics Penicillin G (100 Units/mL) and Streptomycin (100 μg/mL) (Sigma, St Louis, MO, USA) were added to the media to suppress bacterial growth. All plates were incubated in the dark at room temperature for, in average, two weeks until growth of fungal endophyte hyphae from plant tissues was detected.

An inclusive combination of morphological and molecular fungal endophyte identification was employed for identification. Once fungal hyphae were detected growing from the plant material, samples were taken to obtain pure fungal isolates. Genomic DNA was extracted from mycelium of each isolated fungal strain using DNeasy DNA extraction kit (Qiagen, Hilden, Germany) according to the manufacturer's instructions. The endophytes were characterized by the sequences of genomic regions, these sequences are SEQ ID NOs: 26-115. Primers that amplify genomic regions of the endophytes of the present invention are listed in Table 1 (SEQ ID NOs: 1-25). IUPAC nucleotide ambiguity codes are used in the nucleic acid sequences of the present invention.

TABLE 1
Primer sequences useful in identifying
microbes of the present invention

| Primers | Genomic locus |
|---|---|
| ITS_1 (5′-CTTGGTCATTTAGAGGAAGTAA-3′) | ITS |
| (SEQ ID NO: 1) | |
| LR5 (5′-TCCTGAGGGAAACTTCG-3′) | |
| (SEQ ID NO: 4) | |
| LR0R (5′-ACCCGCTGAACTTAAGC-3′) | LSU |
| (SEQ ID NO: 18) | |
| LR5 (5′-TCCTGAGGGAAACTTCG-3′) | |
| (SEQ ID NO: 4) | |
| ITS_2 (5′-GCTGCGTTCTTCATCGATGC-3′) | ITS |
| (SEQ ID NO: 2) | |
| ITS_3 (5′-GCATCGATGAAGAACGCAGC-3′) | |
| (SEQ ID NO: 3) | |
| 60S-506F (5′-CTTVAVYTGGAACTTGATGGT-3′) | 60S ribosomal |
| (SEQ ID NO: 12) | protein L 10 |
| 60S-908R (5′-GHGACAAGCGTTTCTCNGG-3′) | |
| (SEQ ID NO: 13) | |
| PGK (5′-GTYGAYTTCAAYGTYCC-3′) | Phosphoglycerate |
| (SEQ ID NO: 10) | kinase |
| PGK (5′-ACACCDGGDGGRCCGTTCCA-3′) | |
| (SEQ ID NO: 11) | |
| RPB1-Af, largest subunit of RNA polymerase | largest subunit |
| II, primer - amplicon F (5′-GARTGYC | of RNA polymerase |
| CDGGDCAYTTYGG-3′) (SEQ ID NO: 24) | II |
| RPB1-Cr, largest subunit of RNA polymerase | |
| II, primer - amplicon R (5′-CCNGCDAT | |
| NTCRTTRTCCATRTA-3′) (SEQ ID NO: 25) | |
| fRPB2-5F (5′-GAYGAYMGWGATCAYTTYGG-3′) | second largest |
| (SEQ ID NO: 9) | subunit of RNA |
| fRPB2-7R (5′-CCCATWGCYTGCTTMCCCAT-3′) | polymerase II |
| (SEQ ID NO: 8) | |
| bRPB2-7.1R (5′-CCCATRGCYTGYTTMCCCATDGC-3′) | |
| (SEQ ID NO: 7) | |
| Btub2Fd (5′-GTBCACCTYCARACCGGYCARTG-3′) | Partial beta- |
| (SEQ ID NO: 14) | tubulin II |
| Btub4Rd (5′-CCRGAYTGRCCRAARACRAAGTTGTC-3′) | |
| (SEQ ID NO: 15) | |
| ACT512f (5′-ATGTGCAAGGCCGGTTTCG-3′) | Actin |
| (SEQ ID NO: 16) | |
| ACT783r (5′-TACGAGTCCTTCTGGCCCAT-3′) | |
| (SEQ ID NO: 17) | |
| SSU_NS4 (5′-CTTCCGTCAATTCCTTTAAG-3′) | Partial SSU, small |
| (SEQ ID NO: 19) | subunit rRNA gene |
| SSU_NS1 (5′-GTAGTCATATGCTTGTCTC-3′) | |
| (SEQ ID NO: 20) | |
| SSU_NS4 (5′-CTTCCGTCAATTCCTTTAAG-3′) | Partial SSU, small |
| (SEQ ID NO: 19) | subunit rRNA gene |
| SSU SR1R (5′-TACCTGGTTGATCCTGCCAGT-3′) | |
| (SEQ ID NO: 21) | |
| MIC-76091 (5′-GGTGAATCGCACATGCTAGA-3′) | unique genomic |
| (SEQ ID NO: 5) | region |
| MIC-76091 (5′-CGACCAGACAGAGCGTATGA-3′) | |
| (SEQ ID NO: 6) | |
| MIC-68178 (5′-CTCCTCCTCCTCCTCCTGAT-3′) | unique genomic |
| (SEQ ID NO: 22) | region |
| MIC-68178 (5′-TCACAGAGCTACGCGACTTG-3′) | |
| (SEQ ID NO: 23) | |

### Example 2. Identification of Endophytes Using Marker Gene Sequences

Classification of the Fungal Strain Using Marker Gene Sequences Other than ITS

The fungal endophytes of the present invention can be identified by the sequence of one or more of the following loci: second largest subunit of RNA polymerase II (RPB2), 60S ribosomal protein L 10, phosphoglycerate kinase (PGK). PCR amplification of the gene encoding second largest subunit of RNA polymerase II (RPB2) using primer sequences fRPB2-5F (SEQ ID NO: 9) and fRPB2-7.1R (SEQ ID NO: 7) is described in Riess K, Oberwinkler F, Bauer R, Garnica S. High genetic diversity at the regional scale and possible speciation in Sebacina epigaea and S. incrustans. BMC Evolutionary Biology. 2013; 13:102. doi:10.1186/1471-2148-13-102. PCR amplification of the gene encoding second largest subunit of RNA polymerase II (RPB2) using primer sequences fRPB2-5F (SEQ ID NO: 9) and fRPB2-7R (SEQ ID NO: 8) is described in Liu Y, Whelen S, Hall B. Phylogenetic relationships among ascomycetes: evidence from an RNA polymerase II subunit. Mol. Biol. Evol. 1999. 16(12): 1799-1808. PCR amplification of the gene encoding 60S ribosomal protein L 10 using primer sequences 605-506F (SEQ ID NO: 12) and 605-908R (SEQ ID NO: 13) is described in Stielow et al. (2015) One fungus, which genes? Development and assessment of universal primers for potential secondary fungal DNA barcodes, Persoonia 35: 242-263. PCR amplification of the gene encoding Beta-tubulin 2 using primer sequences Btub2Fd (SEQ ID NO: 14) and Btub4Rd (SEQ ID NO: 15) is descriebd in Stielow et al. (2015). PCR amplification of the gene encoding phosphoglycerate kinase using primer sequences PGK_533-F (SEQ ID NO: 10) and PGK_533-R (SEQ ID NO: 11) is described in Stielow et al. (2015). PCR amplification of the SSU using primer sequences SR1R (SEQ ID NO: 21) and NS4 (SEQ ID NO: 19) is described in Zhu et al. (2016) Helminthosporium velutinum and H. aquaticum sp. nov. from aquatic habitats in Yunnan Province, China. Phytotaxa 253 (3): 179-190. PCR amplification of the SSU using primer sequences NS1 (SEQ ID NO: 20) and NS4 (SEQ ID NO: 19) is described in White T. J.; Bruns T.; Lee S. H.; Taylor J. W. PCR protocols: a guide to methods and application. San Diego 1990, 315-32210.1016/B978-0-12-372180-8.50042-1. PCR amplification of Actin using primer sequences ACT512f (SEQ ID NO: 16) and ACT783r (SEQ ID NO: 17) is described in Carbone, I. & Kohn, L. M. (1999) A method for designing primer sets for speciation studies in filamentous ascomycetes. Mycologia, 91(3):552-556. PCR amplification of the largest subunit of RNA polymerase 1 (RPB1) using primer sequences RPB1-Af (SEQ ID NO: 24) and RPB1-Cr (SEQ ID NO: 25) is described in Cendejas-Bueno E, Kolecka A, Alastruey-Izquierdo A, et al. Reclassification of the Candida haemulonii Complex as Candida haemulonii (C. haemulonii Group I), C. duobushaemulonii sp. nov. (C. haemulonii Group II), and C. haemulonii var. vulnera var. nov.: Three Multiresistant Human Pathogenic Yeasts. Journal of Clinical Microbiology. 2012; 50(11):3641-3651.

MIC-76091 can be identified by sequence homology to one or more of the following sequences: second largest subunit of RNA polymerase II (SEQ ID NOs: 53, 55), phosphoglycerate kinase (SEQ ID NO: 54), 60S ribosomal protein L 10 (SEQ ID NO: 56), and a unique genomic region (SEQ ID NO: 57). MIC-67271 can be identified by sequence homology to one or more of the following sequences: second largest subunit of RNA polymerase II (SEQ ID NO: 42), 60S ribosomal protein L 10 (SEQ ID NO: 44), beta-tubulin II (SEQ ID NO: 43), and actin (SEQ ID NO: 45). MIC-68178 can be identified by sequence homology to one or more of the following: beta-tubulin II (SEQ ID NO: 48) and a unique genomic region (SEQ ID NO: 49). MIC-07010 can be identified by sequence homology to SEQ ID NO: 75 which is a partial sequence of the gene encoding phosphoglycerate kinase. MIC-31593 can be identified by sequence homology to one or more of the following: second largest subunit of RNA polymerase II (SEQ ID NO: 79), beta-tubulin II (SEQ ID NO: 80), and a unique genomic region (SEQ ID NO: 81). MIC-96038 can be identified by sequence homology to one or more of the following: actin (SEQ ID NO: 88), beta-tubulin II (SEQ ID NO: 89), second largest subunit of RNA polymerase II (SEQ ID NOs: 90), largest subunit of RNA polymerase II (SEQ ID NO: 91), and a unique genomic region (SEQ ID NO: 92). MIC-33414 can be identified by sequence homology to one or more of the following: actin (SEQ ID NO: 99), large-subunit rRNA (LSU) (SEQ ID NO: 100), largest subunit of RNA polymerase II (SEQ ID NO: 101), small-subunit rRNA (SSU) (SEQ ID NOS:102, 103), beta-tubulin II (SEQ ID NO: 104), and a unique genomic region (SEQ ID NO: 105).

### Classification of the Fungal Strain Using ITS Sequences

Total genomic DNA was extracted from individual fungal isolates, using the DNeasy Plant Mini Kit (Qiagen, Germantown, MD). Polymerase Chain Reaction (PCR) was used to amplify a genomic region including the nuclear ribosomal internal transcribed spacers (ITS) using a primer pair ITS_1 (5′-CTTGGTCATTTAGAGGAAGTAA-3′) (SEQ ID NO: 1) and LR5 (5′-TCCTGAGGGAAACTTCG-3′) (SEQ ID NO: 4). Each 25 microliter-reaction mixture included 22.5 microliters of Invitrogen Platinum Taq supermix, 0.5 microliter of each primer (10 uM), and 1.5 microliters of DNA template (˜2-4 ng). Cycling reactions were run with MJ Research PTC thermocyclers and consisted of 94° C. for 5 min, 35 cycles of 94° C. for 30 s, 54° C. for 30 s, and 72° C. for 1 min, and 72° C. for 10 min. Sanger sequencing of was performed at Genewiz (South Plainfield, NJ) using primers: ITS_1 (5′-CTTGGTCATTTAGAGGAAGTAA-3′) (SEQ ID NO: 1), ITS_2 (5′-GCTGCGTTCTTCATCGATGC-3′) (SEQ ID NO: 2), ITS_3 (5′-GCATCGATGAAGAACGCAGC-3′) (SEQ ID NO: 3), and LR5 (5′-TCCTGAGGGAAACTTCG-3′) (SEQ ID NO: 4). Sequencing primers were chosen so that overlapping regions were sequenced. Raw chromatograms were converted to sequences, and corresponding quality scores were assigned using TraceTuner v3.0.6beta (U.S. Pat. No. 6,681,186). These sequences were quality filtered, aligned and a consensus sequence generated using Geneious v 8.1.8 (Biomatters Limited, Auckland NZ).

Taxonomic classifications were assigned to the sequences using the highest probability of assignment based on the results of industry standard taxonomic classification tools: LCA (runs USEARCH (Edgar, R. C. (2010) Bioinformatics. 26(19):2460-2461) with option search_global, then for all best match hits, returns lowest taxonomic rank shared by all best hits for a query), SPINGO (Allard et al. (2015) BMC Bioinformatics. 16: 324), and UTAX (Edgar, R. C., 2016), using the WARCUP Fungal ITS trainset 1 (Deshpande et al. (2016) Mycologia 108(1):1-5) and UNITE (Koljalg et al. (2013) Molecular Ecology, 22: 5271-5277). The classifier and database combinations listed in Table 2 were used to assign taxonomy to fungal sequences. Taxonomic assignments for endophytes of the present invention are listed in Table 3.

TABLE 2
The classifier and database combinations
used to classify ITS sequences

| Classifier | Database |
|---|---|
| LCA | UNITE, Fungal ITS trainset Jul. 4, 2014 |
| RDP | UNITE, Fungal ITS trainset Jul. 4, 2014 |
| | WARCUP, Fungal ITS trainset 1 |
| SPINGO | UNITE, Fungal ITS trainset Jul. 4, 2014 |
| UTAX | UNITE, Fungal ITS trainset Jul. 4, 2014 |
| | WARCUP, Fungal ITS trainset 1 |

TABLE 3
Exemplary taxonomy and microbe identifiers for fungal endophytes useful in the present invention.

| SEQ | | | | | | | | |
|---|---|---|---|---|---|---|---|---|
| ID | MIC ID | TAMU ID | Phylum | Class | Order | Family | Genus | Species |
| 26, | MIC-91347 | TAM00169 | Ascomycota | Dothideomycetes | Capnodiales | Cladosporiaceae | Cladosporium | cladosporioides |
| 27 | | | | | | | | |
| 28 | MIC-12927 | TAM00193 | Ascomycota | Dothideomycetes | Capnodiales | Cladosporiaceae | Cladosporium | cladosporioides |
| 29 | MIC-31246 | TAM00501 | Ascomycota | Dothideomycetes | Capnodiales | Cladosporiaceae | Cladosporium | |
| 30 | MIC-72531 | TAM00517 | Ascomycota | Dothideomycetes | Capnodiales | Cladosporiaceae | Cladosporium | cladosporioides |
| 31 | MIC-80602 | TAM00249 | Ascomycota | Dothideomycetes | Capnodiales | Cladosporiaceae | Cladosporium | gossypiicola |
| 32 | MIC-42406 | TAM00190 | Ascomycota | Dothideomycetes | Capnodiales | Cladosporiaceae | Cladosporium | herbarum |
| 33 | MIC-87929 | TAM00415 | Ascomycota | Dothideomycetes | Capnodiales | Cladosporiaceae | Cladosporium | herbarum |
| 34 | MIC-50414 | TAM00534 | Ascomycota | Dothideomycetes | Capnodiales | Cladosporiaceae | Cladosporium | oxysporum |
| 35 | MIC-29439 | TAM00201 | Ascomycota | Dothideomycetes | Capnodiales | Cladosporiaceae | Cladosporium | |
| 36 | MIC-10602 | TAM00248 | Ascomycota | Dothideomycetes | Capnodiales | Cladosporiaceae | Cladosporium | |
| 37 | MIC-17794 | TAM00304 | Ascomycota | Dothideomycetes | Capnodiales | Cladosporiaceae | Cladosporium | |
| 38 | MIC-91557 | TAM00463 | Ascomycota | Dothideomycetes | Capnodiales | Cladosporiaceae | Cladosporium | |
| 39 | MIC-26952 | TAM00494 | Ascomycota | Dothideomycetes | Capnodiales | Cladosporiaceae | Cladosporium | |
| 40 | MIC-63740 | TAM00504 | Ascomycota | Dothideomycetes | Capnodiales | Mycosphaerellaceae | Sphaerulina | pseudovirgaureae |
| 41, | MIC-67271 | TAM00089 | Ascomycota | Dothideomycetes | Pleosporales | Didymellaceae | Epicoccum | nigrum |
| 42, | | | | | | | | |
| 43, | | | | | | | | |
| 44, | | | | | | | | |
| 45 | | | | | | | | |
| 46, | MIC-68178 | TAM00032 | Ascomycota | Dothideomycetes | Pleosporales | Didymellaceae | Epicoccum | nigrum |
| 47, | | | | | | | | |
| 48, | | | | | | | | |
| 49 | | | | | | | | |
| 50 | MIC-85555 | TAM00074 | Ascomycota | Dothideomycetes | Pleosporales | Didymellaceae | Epicoccum | nigrum |
| 51 | MIC-77047 | TAM00100 | Ascomycota | Dothideomycetes | Pleosporales | Didymellaceae | Epicoccum | nigrum |
| 52 | MIC-62081 | TAM00103 | Ascomycota | Dothideomycetes | Pleosporales | Didymellaceae | Epicoccum | nigrum |
| 53, | MIC-76091 | TAM00194 | Ascomycota | Dothideomycetes | Pleosporales | Didymellaceae | Epicoccum | nigrum |
| 54, | | | | | | | | |
| 55, | | | | | | | | |
| 56, | | | | | | | | |
| 57, | | | | | | | | |
| 58 | | | | | | | | |
| 59 | MIC-90504 | TAM00497 | Ascomycota | Dothideomycetes | Pleosporales | Didymellaceae | Epicoccum | nigrum |
| 60 | MIC-16066 | TAM00536 | Ascomycota | Dothideomycetes | Pleosporales | Didymellaceae | Epicoccum | nigrum |
| 61 | MIC-90694 | TAM00046 | Ascomycota | Dothideomycetes | Pleosporales | Didymellaceae | | |
| 62 | MIC-20571 | TAM00160 | Ascomycota | Dothideomycetes | Pleosporales | Massarinaceae | Stagonospora | |
| 63 | MIC-26235 | TAM00452 | Ascomycota | Dothideomycetes | Pleosporales | Pleosporaceae | Alternaria | eichhorniae |
| 64 | MIC-39830 | TAM00129 | Ascomycota | Dothideomycetes | Pleosporales | Pleosporaceae | Alternaria | eichhorniae |
| 65 | MIC-31674 | TAM00416 | Ascomycota | Dothideomycetes | Pleosporales | Pleosporaceae | Alternaria | eichhorniae |
| 66 | MIC-61920 | TAM00529 | Ascomycota | Dothideomycetes | Pleosporales | Pleosporaceae | Alternaria | eichhorniae |
| 67 | MIC-39233 | TAM00323 | Ascomycota | Dothideomycetes | Pleosporales | Pleosporaceae | Alternaria | planifunda |
| 68 | MIC-92234 | TAM00013 | Ascomycota | Dothideomycetes | Pleosporales | Pleosporaceae | Bipolaris | spicifera |
| 69 | MIC-77538 | TAM00439 | Ascomycota | Dothideomycetes | Pleosporales | Pleosporaceae | Bipolaris | spicifera |
| 70 | MIC-45943 | TAM00362 | Ascomycota | Dothideomycetes | Pleosporales | Pleosporaceae | Stemphylium | herbarum |
| 71 | MIC-32308 | TAM00473 | Ascomycota | Dothideomycetes | Pleosporales | Pleosporaceae | Stemphylium | herbarum |
| 72 | MIC-12207 | TAM00296 | Ascomycota | Dothideomycetes | Pleosporales | Pleosporaceae | | |
| 73 | MIC-17815 | TAM00518 | Ascomycota | Dothideomycetes | Pleosporales | Sporormiaceae | Preussia | africana |
| 74, | MIC-07010 | TAM00105 | Ascomycota | Dothidiomycetes | Pleosporales | Pleosporaceae | Curvularia | protuberata |
| 75, | | | | | | | | |
| 76 | | | | | | | | |
| 77, | MIC-31593 | TAM00189 | Ascomycota | Dothideomycetes | Pleosporales | Pleosporaceae | Curvularia | spicifera |
| 78, | | | | | | | | |
| 79, | | | | | | | | |
| 80, | | | | | | | | |
| 81 | | | | | | | | |
| 82 | MIC-50324 | TAM00413 | Ascomycota | Eurotiomycetes | Eurotiales | Aspergillaceae | Penicillium | |
| 83 | MIC-95013 | TAM00526 | Ascomycota | Sordariomycetes | Diaporthales | Valsaceae | Phomopsis | liquidambari |
| 84 | MIC-42665 | TAM00524 | Ascomycota | Sordariomycetes | Glomerellales | Plectosphaerellaceae | Gibellulopsis | nigrescens |
| 85 | MIC-16714 | TAM00531 | Ascomycota | Sordariomycetes | Glomerellales | Plectosphaerellaceae | Gibellulopsis | nigrescens |
| 86 | MIC-49739 | TAM00533 | Ascomycota | Sordariomycetes | Glomerellales | Plectosphaerellaceae | Gibellulopsis | nigrescens |
| 87, | MIC-96038 | TAM00505 | Ascomycota | Sordariomycetes | Hypocreales | Hypocreaceae | Acremonium | alternatum |
| 88, | | | | | | | | |
| 89, | | | | | | | | |
| 90, | | | | | | | | |
| 91, | | | | | | | | |
| 92 | | | | | | | | |
| 93 | MIC-87502 | TAM00340 | Ascomycota | Sordariomycetes | Hypocreales | Nectriaceae | Fusarium | |
| 94 | MIC-21610 | TAM00424 | Ascomycota | Sordariomycetes | Hypocreales | Ophiocordycipitaceae | Purpureocillium | lavendulum |
| 95 | MIC-50989 | TAM00490 | Ascomycota | Sordariomycetes | Sordariales | Cephalothecaceae | Phialemonium | inflatum |
| 96 | MIC-39959 | TAM00333 | Ascomycota | Sordariomycetes | Sordariales | Chaetomiaceae | Chaetomium | coarctatum |
| 97, | MIC-33414 | TAM00554 | Ascomycota | Sordariomycetes | Sordariales | Chaetomiaceae | Chaetomium | globosum |
| 98, | | | | | | | | |
| 99, | | | | | | | | |
| 100, | | | | | | | | |
| 101, | | | | | | | | |
| 102, | | | | | | | | |
| 103, | | | | | | | | |
| 104, | | | | | | | | |
| 105 | | | | | | | | |
| 106 | MIC-22947 | TAM00559 | Ascomycota | Sordariomycetes | Sordariales | Chaetomiaceae | Chaetomium | globosum |
| 107 | MIC-44512 | TAM00560 | Ascomycota | Sordariomycetes | Sordariales | Chaetomiaceae | Chaetomium | globosum |
| 108 | MIC-51742 | TAM00251 | Ascomycota | Sordariomycetes | Sordariales | Chaetomiaceae | Chaetomium | piluliferum |
| 109 | MIC-39772 | TAM00317 | Ascomycota | Sordariomycetes | Sordariales | Chaetomiaceae | Chaetomium | |
| 110 | MIC-72092 | TAM00508 | | | | | | |
| 111 | MIC-67609 | TAM00512 | Basidiomycota | Tremellomycetes | Tremellales | Incertae sedis | Cryptococcus | |
| 112 | MIC-39051 | TAM00514 | Basidiomycota | Tremellomycetes | Tremellales | Incertae sedis | Cryptococcus | |
| 113 | MIC-38632 | TAM00489 | | | | | | |
| 114 | MIC-48747 | TAM00244 | Ascomycota | Dothideomycetes | Capnodiales | Cladosporiaceae | Cladosporium | |
| 115 | MIC-20835 | TAM00565 | Ascomycota | Dothideomycetes | Capnodiales | Cladosporiaceae | Cladosporium | |

### Example 3: Preparation of Fungal Biomass and Seed Treatment

Fungal biomass production and heterologous disposition on seeds: Agar plugs of each fungal endophyte (5×5 mm) were transferred to 400 mL Potato Dextrose Broth (PDB; penicillin 10 IU mL⁻¹, streptomycin sulfate 0.1 mg mL⁻¹) in 1 liter flasks placed onto a rotary shaker at 150 rpm under 25-27° C. for two to three weeks. Fungal biomass was harvested from the liquid culture media by straining through several layers of sterile cheesecloth and transferring to 50 mL conical tubes. Fresh biomass was lyophilized under −85° C. using the Labconco® FreeZone 6 (Kansas City, MO, USA) plus for at least 48 hrs. Dry biomass was then manually ground using autoclaved mortar and pestle with dry ice and then kept refrigerated at 4° C.

Dry powdered biomass (50 mg mL⁻¹) was mixed with 1 mL methylcellulose solution (2%) as a sticker and applied to seeds at a rate of 1 mL per 200 seeds. Seeds were air-dried on aluminum trays in a laminar flow hood, occasionally mixed to ensure homogeneous coating on each seeds, and then coated with 1 g talc per 200 seeds to prevent sticking. Formulation control seeds were similarly treated, but without the addition of fungal biomass.

Fungal spore production and heterologous disposition on seeds: Fungal isolates were grown on potato dextrose agar (PDA) for four days, 2 plugs were macerated in 0.05% Silwet with 2-3 3 mm glass beads and the resulting suspension plated onto malt extract agar (MEA) slants in 50 mL conical tubes which were then incubated in a 26° C. growth chamber with 16 hour daylight for 17 days. Spores were harvested by scraping cultures flooded with 0.05% Silwet and filtering the resulting suspension through a 60 μm nylon membrane. Spores were quantified with a CytoFlex Flow Cytometer and serial dilutions of the spore suspension were plated onto PDA to quantify the proportion of viable spores.

Fungal spore suspensions were added to seeds at a normalized dose rate of 6×10\hat{ }4 spores per seed. Treated seeds were then coated with a flowability polymer. Control seeds received 0.05% Silwet solution and flowability polymer without spores. On surface spore viability was assessed by agitating treated seeds in 40 mM sodium phosphate buffer and plating serial dilutions of the resulting suspension onto PDA.

### Example 4: Greenhouse Cotton Time to Wilt and Time to Death

Relatively small increases of one or two days in seedling time to wilt or time to death under water stress have a substantial and agronomically relevant impact on seedling establishment and cotton stand. Among other things, this example describes a greenhouse assay that mimics environmental conditions of extended water stress during the seedling stage of plant development in field production of cotton. Among other things, this example describes strains of fungal endophytes that provide an improved response to water stress to treated cotton plants.

Seed inoculation: Black cotton seeds of varieties Phytogen 499WRF and Delta Pine 1321B2RF were treated with fungal endophyte biomass prepared as described in Example 3. Dry powdered biomass (50 mg mL⁻¹) was mixed with 1 mL methylcellulose solution (2%) as a sticker and applied to seeds at a rate of 1 mL per 200 seeds. Seeds were air-dried on aluminum trays in a laminar flow hood, occasionally mixed to ensure homogeneous coating on each seeds, and then coated with 1 g talc per 200 seeds to prevent sticking. Formulation control seeds were similarly treated, but without the addition of fungal biomass.

Plant production: Seeds of each treatment combination (inoculated or control seeds) were planted individually in seedling germination trays. Each cell pot measured 4 cm top diameter×6 cm deep and was filled with nonsterile Metro-Mix® 900 soil (Sun Gro Horticulture, Agawam, MA; ingredients: bark, vermiculite, peat moss, perlite, dolomitic limestone) watered to saturation prior to planting. Plants were grown in a controlled temperature room at 25° C. under constant overhead illumination (EnviroGro T5 High Output Fluorescent Lighting Systems). Equal amounts of water corresponding to the pot saturation volume were applied to each plant at 7 and 14 days after planting (DAP) by which time they had reached the early 1^{st} true leaf stage. Water was then withheld from all the endophyte-treated and control plants which were monitored daily for the onset of wilting and day of death. Both the time to event, i.e. the day within the evaluation period at which either wilting or death occurred, and the event status, i.e. a binary tally of whether or not the event occurred, were recorded. Tray positions were randomized and rotated daily to control for potential position effects.

Data analysis: The survival package (v. 2.40-1) (Therneau T (2015). A Package for Survival Analysis in S. version 2.38, available online at CRAN.R-project.org/package=survival; Terry M. Therneau and Patricia M. Grambsch (2000). Modeling Survival Data: Extending the Cox Model. Springer, New York. ISBN 0-387-98784-3.) in R (v. 3.2.2) (R Development Core Team (2008). R: A language and environment for statistical computing. R Foundation for Statistical Computing, Vienna, Austria. ISBN 3-900051-07-0, available online at R-project.org.) was used to run the Cox proportional hazards model to generate hazard ratios (HR) and associated p-values for each strain compared to the formulation controls within the same experiment. Both the strain used as a seed treatment and the crop variety were included in the model: coxph(Surv(time to event, event status)˜strain+variety)

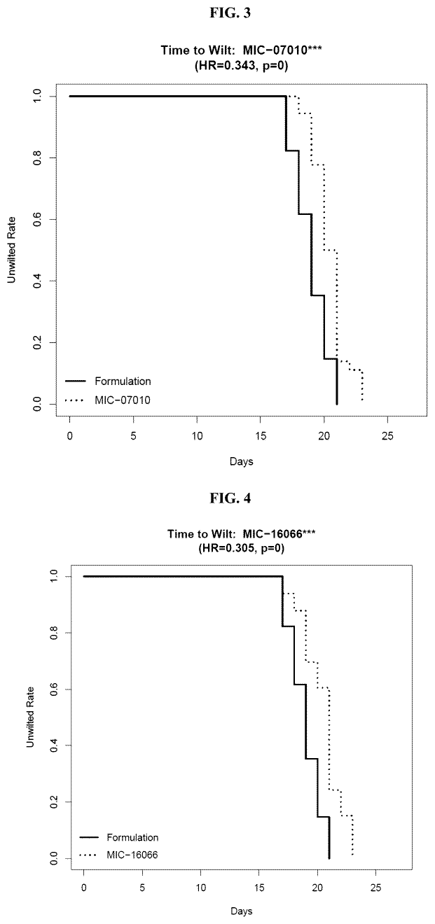

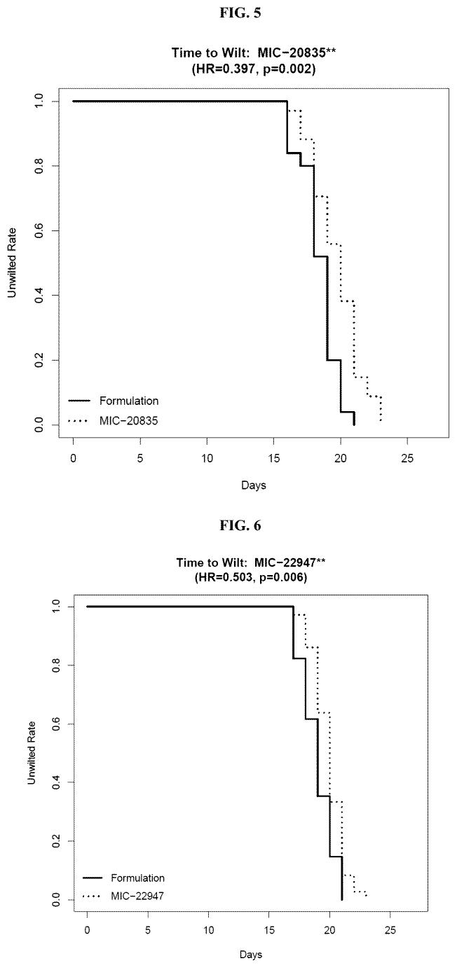

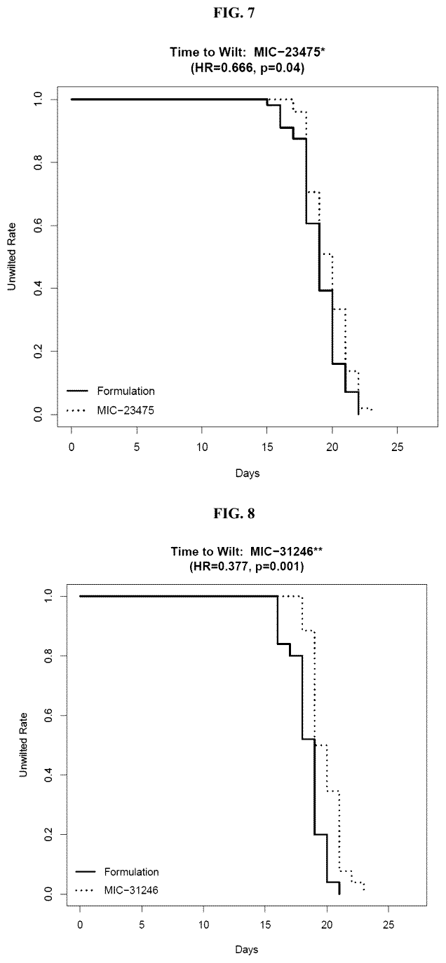

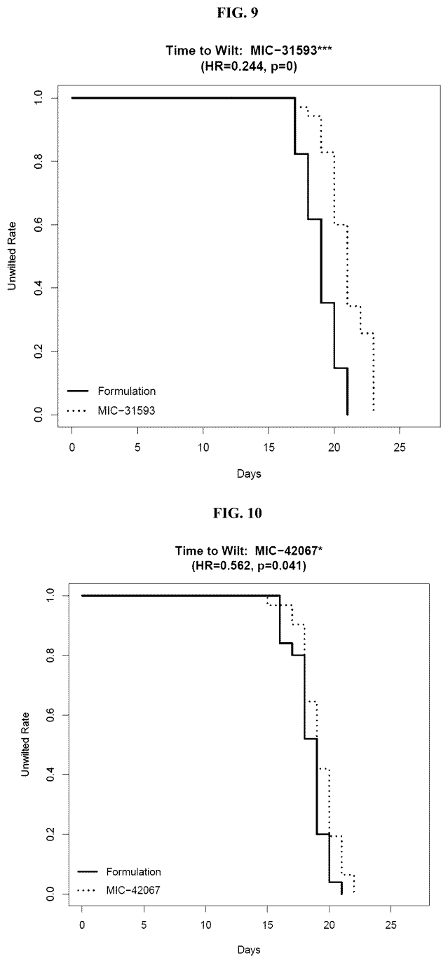

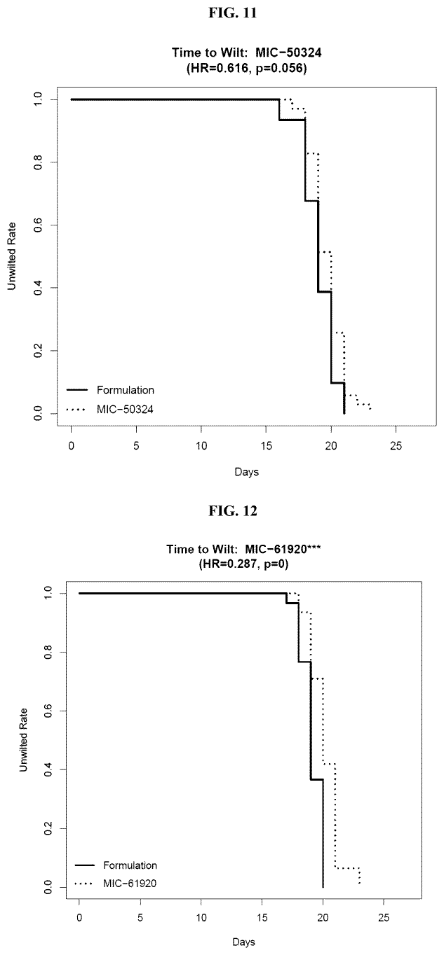

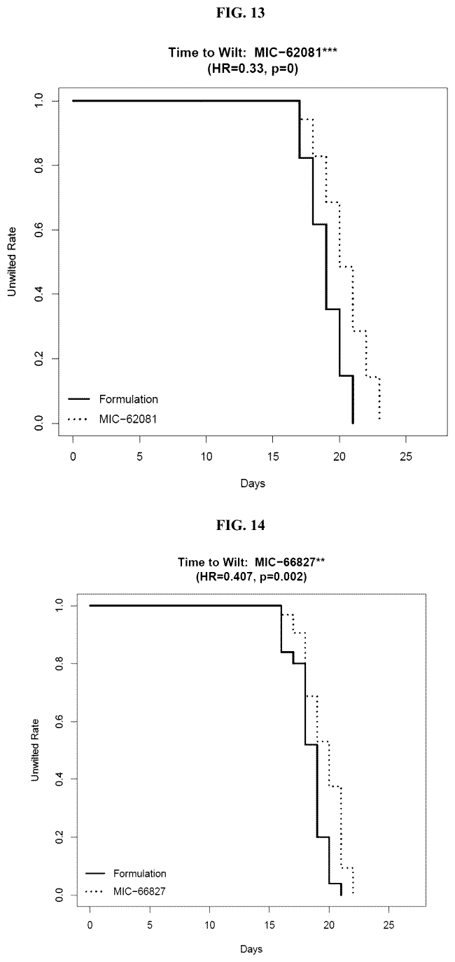

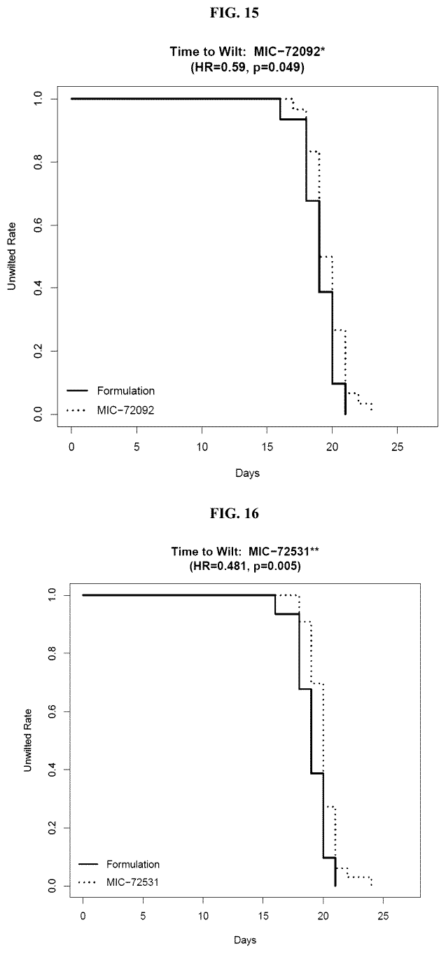

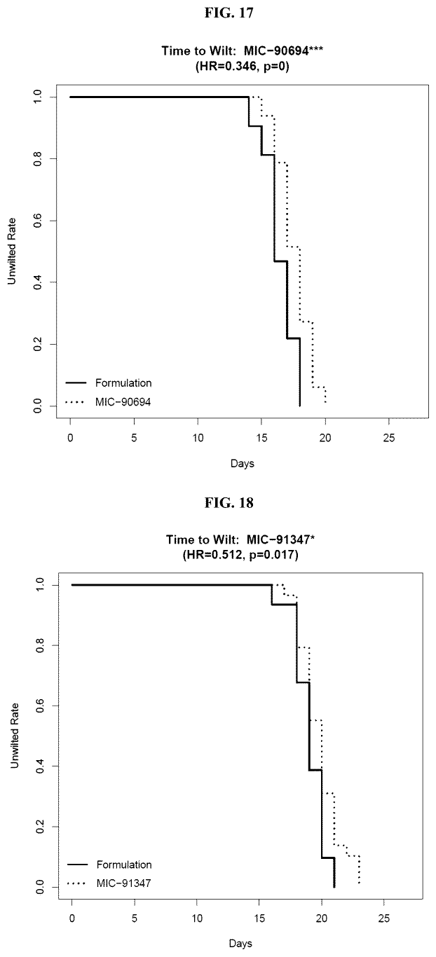

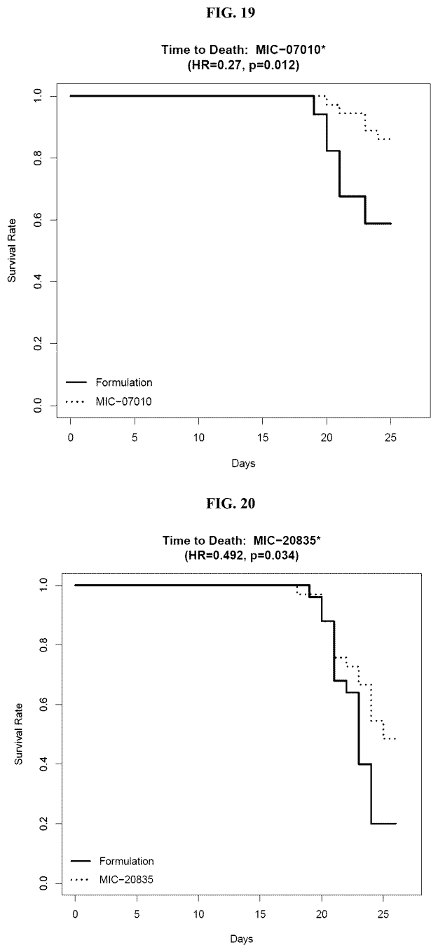

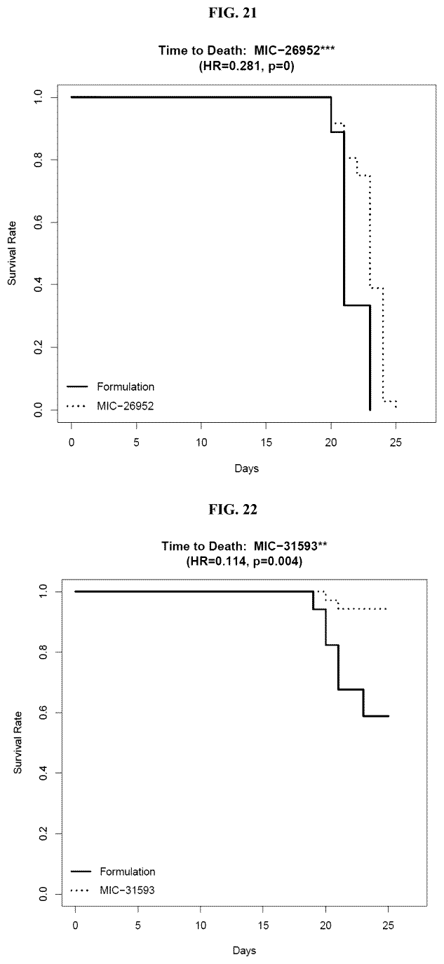

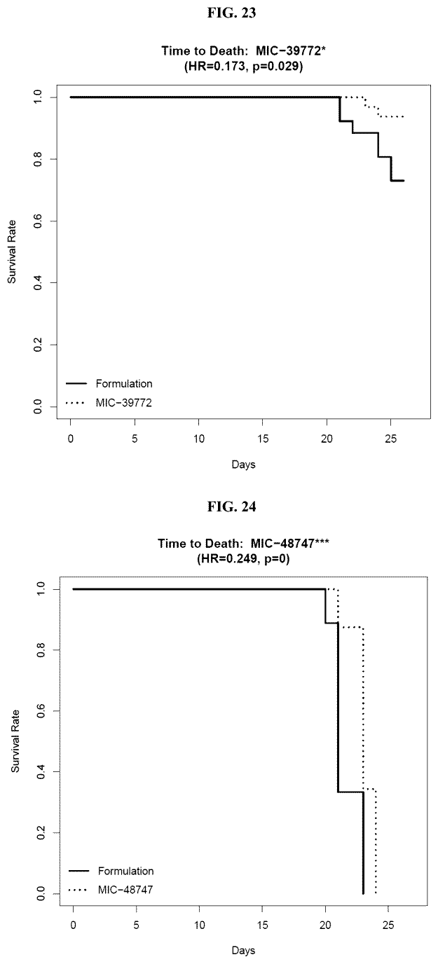

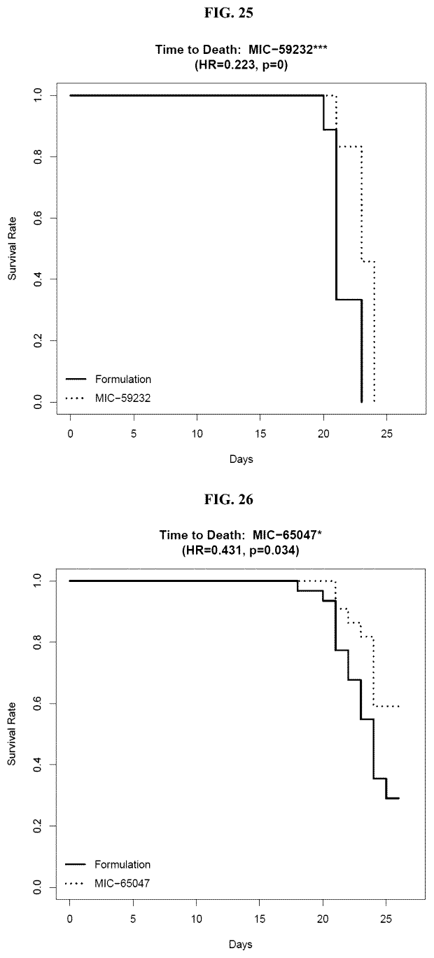

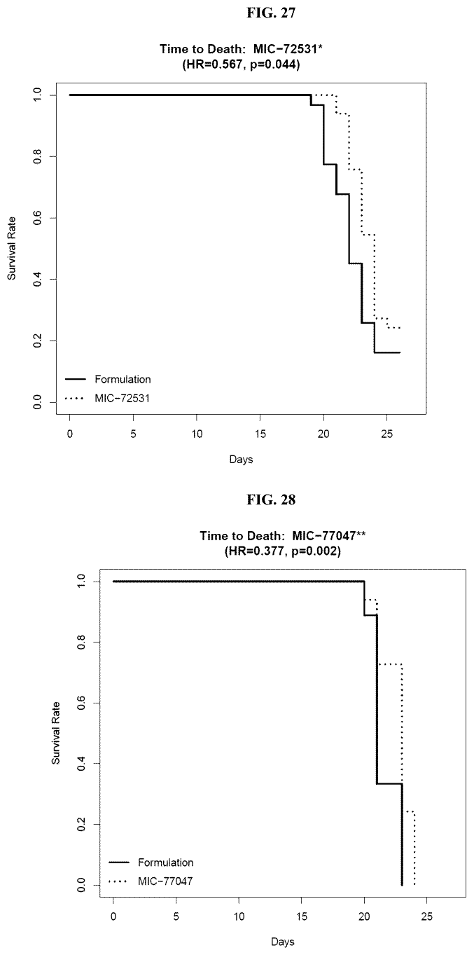

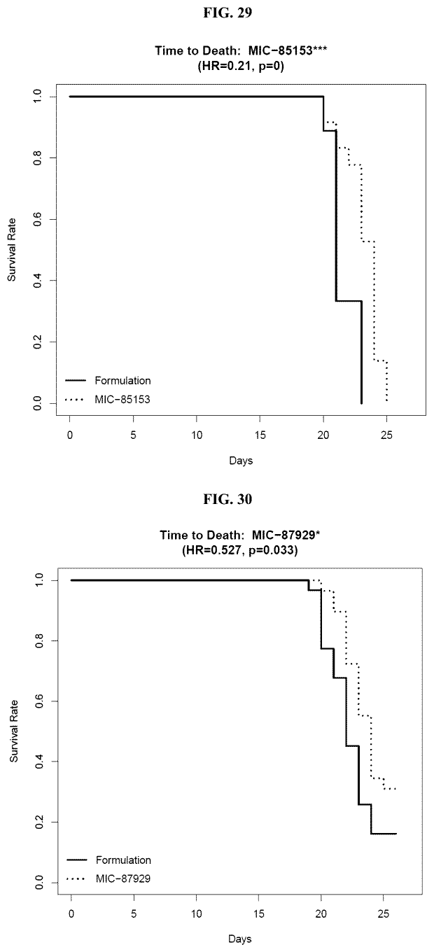

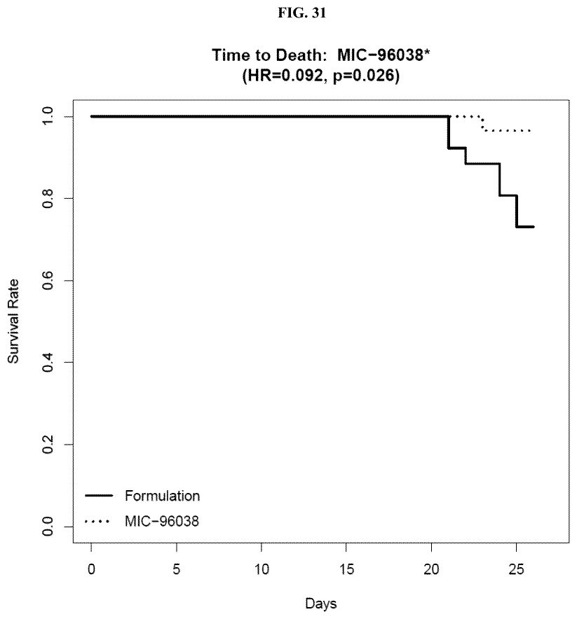

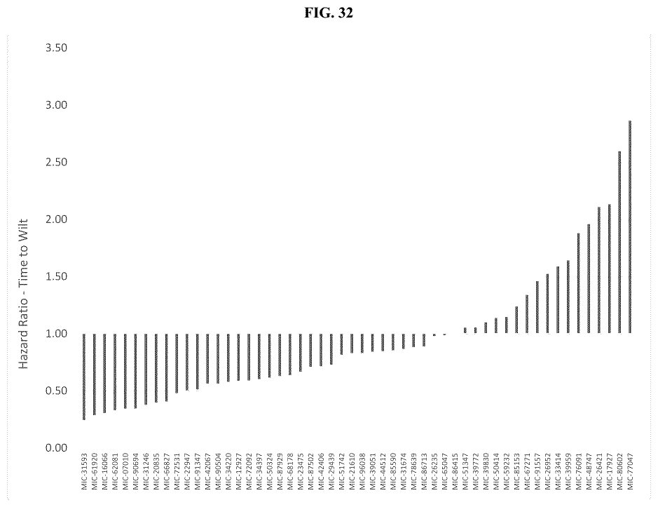

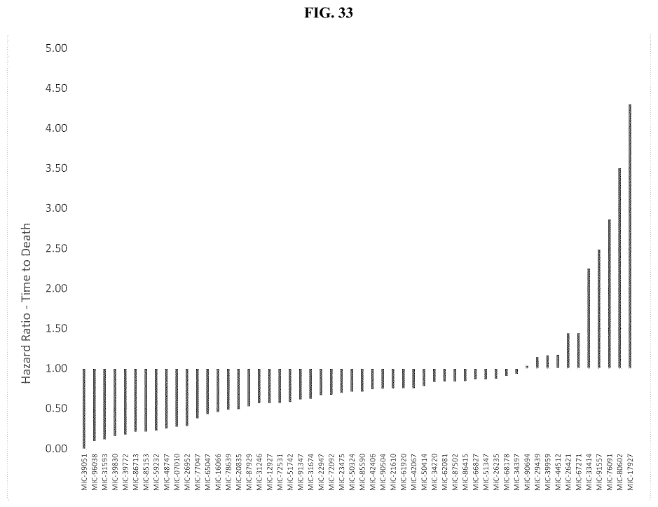

Hazard ratios >1 indicate that the endophyte treated plants experience a higher risk of the modeled hazard (wilt or death) and a lower survival rate under water stress compared to formulation controls. Conversely, hazard ratios <1 indicate that the endophyte treated plants experience a lower risk of the modeled hazard (wilt or death) and a higher survival rate under water stress compared to formulation controls (FIGS. 32-34). Kaplan Meier survival curves were generated for each strain using the rms package (v. 5.0-0) (Frank E Harrell Jr (2016). rms: Regression Modeling Strategies. R package version 5.0-0. available online at CRAN.R-project.org/package=rms) in R. Exemplary Kaplan Meier survial curves for exemplary endophyte treated plants relative to formulation controls are shown in FIGS. 3-31, hazard ratios are summarized in FIGS. 32-34.

Results: For time to wilt, 15 of 54 strains evaluated showed a statistically significant increase in time to wilt under water stress compared to formulation control treated plants (Table 4). For time to death, 18 of 54 strains evaluated showed a statistically significant increase in time to death under water stress compared to formulation control treated plants (Table 5); these endophytes may be identified by sequence homology to one or more sequence selected from the group consisting of SEQ ID NOs: 30, 33, 39, 51, 64, 74, 75, 76, 77, 78, 79, 80, 81, 87, 88, 89, 90, 91, 92, 109, and 114.

TABLE 4
Of 54 strains evaluated as seed treatments for two varieties of cotton,
15 showed a statistically significant increase in time to wilt under
water stress compared to formulation control treated plants.

| | | | Pr (>\|z\|) |
|---|---|---|---|
| Taxonomic ID | Strain ID | HR Wilt | Wilt |
| Didymellaceae | MIC-90694 | 0.35 | 0.000173 |
| Epicoccum nigrum | MIC-62081 | 0.33 | 6.63E−05 |
| Curvularia protuberata | MIC-07010 | 0.34 | 3.90E−05 |
| Chaetomium sp. | MIC-66827 | 0.41 | 0.002422 |
| Chaetomium globosum | MIC-23475 | 0.67 | 0.040085 |
| Diaporthe sp. | MIC-42067 | 0.56 | 0.040736 |
| Cladosporium cladosporioides | MIC-91347 | 0.51 | 0.016866 |
| Alternaria sp. | MIC-86713 | 0.46 | 0.006176 |
| Curvularia spicifera | MIC-31593 | 0.24 | 1.11E−06 |
| Cladosporium sp. | MIC-31246 | 0.38 | 0.001339 |
| Cladosporium cladosporioides | MIC-72531 | 0.48 | 0.004828 |
| Alternaria eichhorniae | MIC-61920 | 0.29 | 0.000123 |
| Epicoccum nigrum | MIC-16066 | 0.31 | 2.73E−05 |
| Chaetomium globosum | MIC-22947 | 0.50 | 0.005774 |
| Cladosporium cladosporioides | MIC-20835 | 0.40 | 0.002042 |

TABLE 5
Of 54 strains evaluated as seed treatments for two varieties of cotton,
15 showed a statistically significant increase in time to death under
water stress compared to formulation control treated plants.

| Taxonomic ID | Strain ID | HR Death | Pr(>\|z\|) Death |
|---|---|---|---|
| Epicoccum nigrum | MIC-77047 | 0.38 | 0.002237 |
| Curvularia protuberata | MIC-07010 | 0.27 | 0.012087 |
| Epicoccum nigrum | MIC-65047 | 0.43 | 0.033795 |
| Alternaria eichorniae | MIC-39830 | 0.15 | 2.71E−06 |
| Alternaria sp. | MIC-86713 | 0.21 | 0.000604 |
| Curvularia spicifera | MIC-31593 | 0.11 | 0.004091 |
| Cladosporium sp. | MIC-48747 | 0.25 | 3.94E−05 |
| Chaetomium sp. | MIC-39772 | 0.17 | 0.028894 |
| Chaetomium globosum | MIC-59232 | 0.22 | 9.32E−05 |
| Cladosporium herbarum | MIC-87929 | 0.53 | 0.032516 |
| Epicoccum nigrum | MIC-85153 | 0.21 | 1.39E−05 |
| Cladosporium sp. | MIC-26952 | 0.28 | 0.000146 |
| Acremonium alternatum | MIC-96038 | 0.09 | 0.02573 |
| Cladosporium cladosporioides | MIC-72531 | 0.57 | 0.044338 |
| Cladosporium cladosporioides | MIC-20835 | 0.49 | 0.033986 |

### Example 5. In Vitro Antibiosis

This example describes an exemplary method of in vitro antibiosis screenings of microbes against the crop pathogen Fusarium oxysporum, using the non-pathogenic Fusarium oxysporum Fo47 (ATCC, MYA-1198). Caspofungin diacetate (Sigma, SML0425-5MG) is a compound with antifungal activity that is used as a positive control. Caspofungin inhibits 13-1,3-D-glucan synthase and thereby disrupting fungal cell wall integrity. Amphotericin B is a compound with antifungal activity that is used as a positive control. All stock compounds are prepared in DMSO at a concentration of 5,120 μg/ml.

### Preparation of Fo47 Spores

Fo47 is cultured on 2% potato dextrose agar (PDA) plates for 14 days at room temperature in a weak light condition. Three ml of 0.05% Silwett L-77 in 1× phosphate buffered saline (PBS) is added to each plate, then mycelium are scraped off and filtered through glass wool into a new 50 ml Falcon tube. Spores are then counted using a hemocytometer and adjusted to 5×106 CFU/ml with sterile 1×PBS.

### Preparation of Endophytic Fungal Culture

Five glass beads (3 mm) are added to each well of a 24-deep well plate (VWR, Cat. No. 89080-534) and autoclaved. Fungal cultures are started by adding 5 μl of spore suspension normalized to 1×106 cfu/ml into 3 ml PDB culture into each well. The plates are incubated for 3 days at room temperature with vigorous shaking at 500 rpm.

### Antibiosis Assay

Prepare PDA plates: PDA with 1% agar are autoclaved in a liquid cycle for 20 minutes with a magnetic stir bar in the flask and kept in a 50° C. water bath. When ready the PDA flask is taken to a sterile environment such as a biosafety cabinet and cooled at room temperature for 15-20 min. Then 2 ml of the prepared Fusarium spores are added per 1 liter of PDA. OmniTrays (ThermoFisher, Cat. No. 264728) are filled with 60 ml of the PDA/spore mixture. After the plates solidify, the plates are air dried for 30 min before covering with the lid.

For each OmniTray, 24 wells are drilled at once using the liquid handling system, BioMek Fx with the following setting: load podl (96 pin head) with 24 200-μl wide bore barrier tips (Beckman Coulter, Cat. No. B01110-AA), draw 165 μl well contents using the “Bacterial culture 100 μl technique” at 1.5 mm from the bottom of OmniTray using the “override technique”, dispense tips contents to reservoir plate using the “Reservoir technique” at 6 mm from bottom of OmniTray using “override technique”.

For each OmniTray, 7.5 μl of the prepared bacterial cultures are added into each of the 24 wells using BioMek Fx system, 3 replicated plates are prepared. A negative control (nothing added), a medium control, a DMSO control, a positive compound control (e.g. Caspofungin diacetate, or Amphotericin B) and a positive biological control of the same volume are included on each plate. The plates are then incubated at room temperature in sterile conditions for 4 days. Photographs are taken of each plate and the zone of inhibition between the cultures and Fusarium growth are qualitatively scored using a 0-3 scale (3 denotes a strong inhibition) and quantitatively measured using the ImageJ program.

### Example 6. Greenhouse Assessment of Improved Plant Characteristics Under Biotic Stress

This example describes an exemplary method of greenhouse screening of microbes against a crop pathogen Rhizoctonia solani, one of the causal agents of seedling damping off disease.

Preparation of Rhizoctonia R9 inoculum

A permanent stock of R9 is maintained on corn meal agar slants at room temperature. R9 is sub-cultured in a PDA plate for a week, then 5 plugs of mycelium are transferred into one liter of PDB broth in a 3-liter flask. The culture is grown at room temperature with vigorous shaking for 5 days. The entire one liter of the culture is poured into and mixed well 4 pounds of doubly autoclaved millet seeds. The mixture is sealed in a large plastic bag and incubated for 2 weeks at room temperature with gentle mixing every other day followed by a 2-day air drying inside a biosafety cabinet. Dried infected millet seeds are aliquoted into smaller bags and are usually used to set up disease assay in greenhouse within a week.

### Greenhouse Assay Setup

This greenhouse assay is conducted in 6.5 inch diameter plastic pots. The pots are first filled with 400 cc of mildly moistured Sunshine potting mix, followed by another layer of 400 cc potting mix uniformly blended with 2 tablespoons of R9-infected millet seeds. The pots are generally two third full with 800 cc of potting mix. The pots are left sitting at room temperature under dark condition for two nights before placing seeds to ensure a thick layer of aggressively grown pathogen mycelium in the soil.

This greenhouse assay is conducted using chemically treated soy seeds coated with fungal endophytes described herein and formulation control (no endophyte) and untreated controls (no endophyte and no formulation) as described in Example 3. Five seeds are evenly placed onto each pot on top of the inoculum layer and the pots are filled up with another 400 cc potting mix. Ten replicated pots of each treatment are set up and placed on a greenhouse bench using a random block design. The following growth and vigor metrics are measured: percentage emergence at Day 7 and percentage standing at Days 14 and 21, top view images at Day 7 and side view images of pulled and washed seedlings at Day 21, plant height at Day 21, plant dry weight at Day 21, and root crown disease rating at Day 21 using a 0-5 scale with 5 denotes the strongest necrosis and collapse of stem at the root crown region.

At Day 21 post planting, seedlings are gently pulled off the pot, washed with tap water to remove dirt, and kept in plastic bags at 4° C. for further data measurement. The severity of root crown necrosis is first independently rated by multiple persons using the scale described above, followed by plant height measurement before being laid on to a fluted plastic board for side view imaging. After side view imaging, seedlings from the same pot are put into a paper bag and dried in an oven. Plant dry weight of each individual seedling is recorded.

### Example 7: Treatment of Nematode Infestation

A two-tiered approach was used to evaluate the efficacy and repeatability of 56 strains of fungi originally isolated as foliar endophytes from cotton (Gossypium hirsutum) for antagonistic effects on root-knot nematodes (Meloidogyne incognita). All fungi were inoculated to cotton using a seed treatment. A majority of the fungi tested had negative effects on root-knot nematode galling three weeks after egg inoculation of cotton seedlings. Across replicated greenhouse assays, 40% percent of the strains exhibited consistent statistically significant negative effects. Strains with consistent negative effects belonged to the genera Chaetomium, Cladosporium, Epicoccum, and Phomopsis. Three strains in the genera Bipolaris, Chaetomium, and Phomopsis had an opposite effect and significantly increased gall numbers. This example describes that a large proportion of cotton fungal endophytes are capable of conferring some degree of resistance to the plant from root-knot nematode infection.

Seed inoculation: Seeds of a nematode susceptible cotton cultivar PhytoGen PHY499WRF were treated with fungal endophyte biomass prepared as described in Example 3. 50 mg of ground dry-biomass was mixed with 1 mL of 2% Methyl cellulose (MC) solution (Sigma-Aldrich®, M7140-250G, 15 cP viscosity), which was finalized to the concentration of 10⁵ CFUs mL⁻¹. Approximately 200 seeds (delinted black seed without fungicides or insecticides) were coated using 1 mL of either the sticker solution alone (Control) or the fungus-containing sticker solution, and then dried at room temperature and finished with talc powder (Sigma-Aldrich®, Prod. No. 18654) to prevent sticking.

Host plants: Inoculated seeds were planted and germinated in pasteurized sand (steamed for eight hours at 72° C.) in seed starter trays (each cell pot measured 4 cm top diameter×6 cm deep) in a plant growth facility at 24° C. (12L: 12D photoperiod) until first true-leaf stage.

Nematode preparation and infection: M. incognita eggs were extracted from infected tomato plants by agitating the roots in 0.6% NaOCl for 4 min, and collected on a sieve with a pore size of 25 μm (Hussey and Barker, 1973). Egg concentration in the extraction solution was quantified under a microscope using a Neubauer hemocytometer (a modified method of Gordon and Whitlock (1939)). Cotton seedlings at the first true-leaf stage were inoculated by pipetting a volume of egg suspension containing approximately 2000 eggs directly to the soil at the base of the plant.

Evaluation of nematode infection: Plants were maintained in the greenhouse for three weeks after nematode inoculation (WAI), then carefully removed from pots and washed free of soil from the roots. Root fresh weight was measured and the total number of galls per root system was quantified for each plant. A total of 15 replicate plants per treatment group were sampled.

A two-tiered approach was used to evaluate the repeatability of observed negative effects on nematode galling. First, an initial series of assays was performed as described herein on all 56 fungal strains. A second series of replicate follow-up assays was then performed on a reduced endophyte set consisting only of strains that exhibited statistically significant reductions in nematode galls in the first assay.

Bioassays were conducted across eight different rounds, each with a corresponding control treatment grown at the same time, in order to cycle all strains through the assay. All comparisons between treatment and control plants were made only among plants grown within the same bioassay round. The strains tested within each round are listed in Table 6.

Statistical analysis: All statistical analyses were performed using IMP® Pro, Version 12.0.1 (SAS Institute Inc., Cary, NC, USA). All data were tested for normality and equality of variances. One-way ANOVA was performed to analyze the impact of endophyte treatment on gall numbers per gram of root tissue (α=0.05). If a significant overall treatment effect was detected, post-hoc Dunnett's tests were used to compare the mean of the control against all the other treatments (α=0.05).

Significant overall effects of fungal treatments on nematode on gall numbers were found within each of the eight separate rounds of bioassays conducted (ANOVA Round 1: F_{4, 70}=7.63, p<0.0001; Round 2: F_{5, 84}=7.10, p<0.0001; Round 3: F_{12, 182}=4.84, p<0.0001; Round 4: F_{10, 154}=10.38, p<0.0001; Round 5: F_{10, 154}=8.93, p<0.0001; Round 6: F_{15, 224}=4.05, p<0.0001; Round 7: F_{11, 168}=16.75, p<0.0001; Round 8: F_{11, 168}=17.38, p<0.0001). Result of pairwise comparisons between the treatment and control groups are reported separately in Table 1 for each of the eight rounds of bioassays conducted.

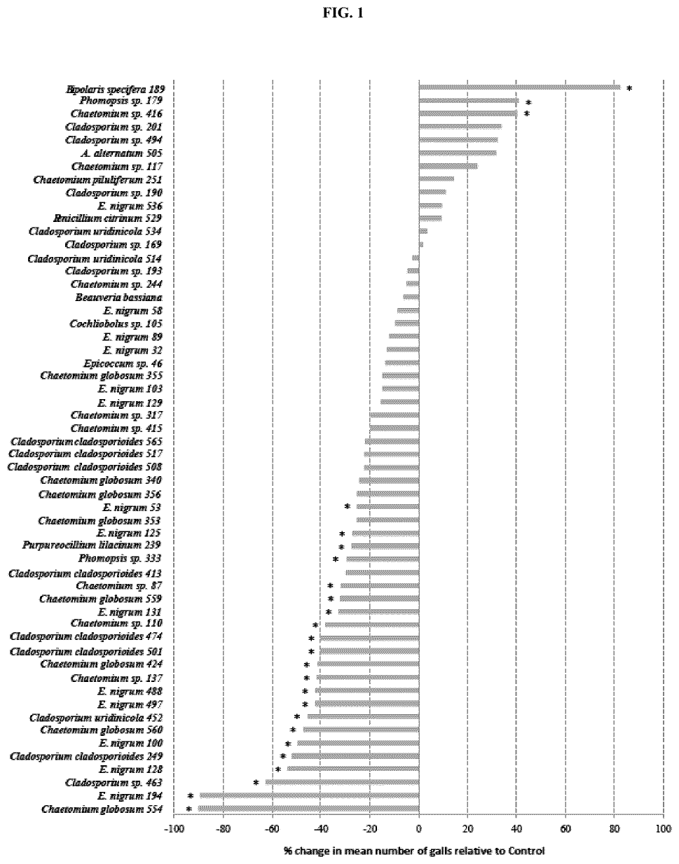

Endophytic fungi from cotton exhibited repeatable negative effects on nematode galling. A majority of the fungi endophytes reduced the number of galls in treated relative to control plants in the first round of assays (FIG. 1). The number of strains with negative effects on nematodes (77%) was significantly higher than would have been expected under the null hypothesis of no effect of the fungal treatments (50%) (Fisher's exact test, p=0.0029). Of the 56 strains initially assayed, 22 (39%) exhibited statistically significant reductions in nematode galling (FIG. 1; Table 6). These 22 strains were selected for further evaluation in the second series of replicate follow-up assays.

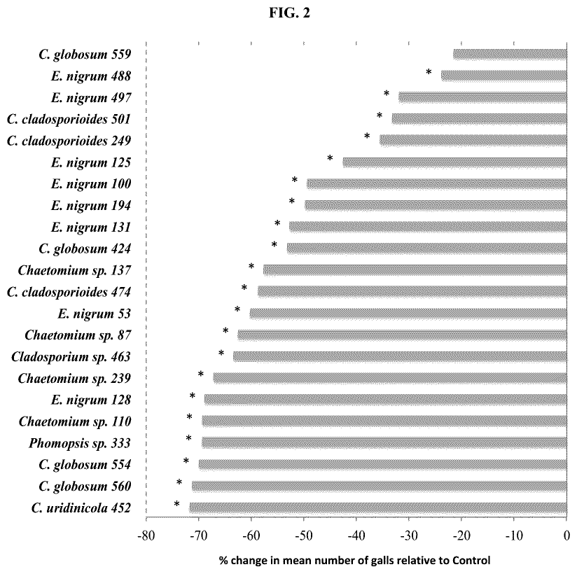

The reductions in nematode galling observed in the first series of assays were highly repeatable. All of strains selected for follow up evaluation in the second series of assays reduced galling relative to the control plants. Of the 22 strains tested, 21 (95%) significantly reduced root-knot nematode galling across both replicate trials (FIG. 2; Table 6). Examples of endophytes which consistently reduced root-knot nematode galling include, those identified by sequence homology to one or more sequence selected from the group consisting of SEQ ID NOs: 29, 31, 38, 51, 53, 54, 55, 56, 57, 58, 59, 63, 94, 96, 97, 98, 99, 100, 101, 102, 103, 104, 105, and 107.

A taxonomic summary of the observed negative and positive effects on nematode galling based on the genera of fungi tested is provided in Table 7.

This example describes that cotton fungal endophytes are capable of conferring some degree of resistance to the plant from root-knot nematode infection. In some embodiments, endophytic fungal strains described herein reduced root-knot nematode galling of cotton seedlings. Importantly, this effect was highly repeatable, with 95% of the isolates that exhibited a significant reduction in galling in the first assay, again doing so in a second replicate assay. To our knowledge, this study provides the first examples negative effects on root-knot nematodes by endophytic isolates of Epicoccum and Phomopsis fungi.

TABLE 6
Number of galls produced by root-knot nematode per
gram of root tissue (mean ± SE) in each treatment.
Statistical differences between treatments and the Control
group were compared using Dunnett's test (α = 0.05)

| Bioassay | Treatment | Mean ± S.E. | P - value |
|---|---|---|---|
| Round 1 | Control | 28.02 ± 2.81 | — |
| | Curvularia spicifera TAMU189 (MIC-31593) | 51.19 ± 6.03 | 0.0002 |
| | Acremonium alternatum TAMU505 (MIC-96038) | 36.97 ± 3.51 | 0.29 |
| | Cladosporium oxysporum TAMU534 (MIC-50414) | 29.01 ± 2.24 | 1.00 |
| | Curvularia protuberata TAMU105 (MIC-07010) | 25.23 ± 3.29 | 0.96 |
| Round 2 | Control | 81.17 ± 14.90 | — |
| | Didymellaceae TAMU46 (MIC-90694) | 69.96 ± 23.48 | 0.94 |
| | Cladosporium gossypiicola TAMU249 (MIC-80602) | 39.01 ± 3.64 | 0.047 |
| | Cladosporium sp. TAMU463 (MIC-91557) | 30.27 ± 2.37 | 0.011 |
| | Epicoccum nigrum TAMU194 (MIC-76091) | 8.47 ± 1.28 | 0.0001 |
| | Chaetomium globosum TAMU554 (MIC-33414) | 8.00 ± 1.24 | 0.0001 |
| Round 3 | Control | 54.76 ± 5.31 | — |
| | Epicoccum nigrum TAMU89 (MIC-67271) | 48.02 ± 4.16 | 0.74 |
| | Epicoccum nigrum TAMU103 (MIC-62081) | 46.57 ± 3.42 | 0.51 |
| | Alternaria sp. TAMU53 (MIC-34397) | 40.77 ± 8.09 | 0.042 |
| | Epicoccum nigrum TAMU125 (MIC-51347) | 39.81 ± 1.96 | 0.024 |
| | Purpureocillium lilacinum TAMU239 (MIC-86415) | 39.71 ± 2.81 | 0.022 |
| | Chaetomium coarctatum TAMU333 (MIC-39959) | 38.48 ± 4.59 | 0.010 |
| | Chaetomium sp. TAMU87 (MIC-78639) | 37.32 ± 3.44 | 0.0047 |
| | Epicoccum nigrum TAMU131 (MIC-85590) | 36.74 ± 2.41 | 0.0031 |
| | Diaporthe sp. TAMU137 (MIC-42067) | 31.76 ± 3.61 | <0.0001 |
| | Epicoccum nigrum TAMU497 (MIC-90504) | 31.54 ± 3.71 | <0.0001 |
| | Alternaria eichhorniae TAMU452 (MIC-26235) | 29.85 ± 2.37 | <0.0001 |
| | Chaetomium globosum TAMU560 (MIC-44512) | 28.94 ± 3.24 | <0.0001 |
| Round 4 | Control | 42.18 ± 4.32 | — |
| | Chaetomium globosum TAMU117 (MIC-23475) | 52.32 ± 4.64 | 0.20 |
| | Chaetomium piluliferum TAMU251 (MIC-51742) | 48.23 ± 3.30 | 0.76 |
| | Beauveria bassiana | 39.49 ± 3.08 | 1.00 |
| | Epicoccum nigrum TAMU58 (MIC-26421) | 38.41 ± 2.95 | 0.98 |
| | Alternaria eichorniae TAMU129 (MIC-39830) | 35.54 ± 4.51 | 0.67 |
| | Chaetomium globosum TAMU356 (MIC-59232) | 31.50 ± 3.36 | 0.16 |
| | Chaetomium globosum TAMU559 (MIC-22947) | 28.57 ± 1.98 | 0.035 |
| | Epicoccum nigrum TAMU488 (MIC-85153) | 24.30 ± 1.58 | 0.0020 |
| | Epicoccum nigrum TAMU100 (MIC-77047) | 21.19 ± 2.88 | 0.0002 |
| | Epicoccum nigrum TAMU128 (MIC-65047) | 19.43 ± 2.66 | <0.0001 |
| Round 5 | Control | 53.08 ± 4.27 | — |
| | Alternaria sp. TAMU179 (MIC-86713) | 74.84 ± 6.00 | 0.018 |
| | Alternaria eichhorniae TAMU416 (MIC-31674) | 74.42 ± 5.62 | 0.021 |
| | Cladosporium sp. TAMU494 (MIC-26952) | 70.22 ± 5.09 | 0.10 |
| | Epicoccum nigrum TAMU536 (MIC-16066) | 58.21 ± 5.61 | 0.99 |
| | Alternaria eichhorniae TAMU529 (MIC-61920) | 57.99 ± 5.38 | 1.00 |
| | Cryptococcus sp. TAMU514 (MIC-39051) | 51.61 ± 5.89 | 1.00 |
| | Cladosporium sp. TAMU244 (MIC-48747) | 50.35 ± 3.57 | 1.00 |
| | Epicoccum nigrum TAMU32 (MIC-68178) | 45.99 ± 4.80 | 0.92 |
| | Chaetomium sp. TAMU110 (MIC-66827) | 32.61 ± 3.56 | 0.030 |
| | Cladosporium cladosporioides TAMU474 (MIC-34220) | 31.86 ± 3.56 | 0.022 |
| Round 6 | Control | 57.47 ± 3.25 | — |
| | Cladosporium sp. TAMU201 (MIC-29439) | 76.87 ± 8.38 | 0.13 |
| | Cladosporium herbarum TAMU190 (MIC-42406) | 63.82 ± 7.13 | 1.00 |
| | Cladosporium cladosporioides TAMU169 (MIC-91347) | 58.46 ± 6.34 | 1.00 |
| | Cladosporium cladosporioides TAMU193 (MIC-12927) | 54.84 ± 8.24 | 1.00 |
| | Nectriaceae TAMU355 (MIC-17927) | 48.91 ± 4.72 | 0.95 |
| | Chaetomium sp. TAMU317 (MIC-39772) | 46.16 ± 3.98 | 0.75 |
| | Cladosporium herbarum TAMU415 (MIC-87929) | 45.99 ± 2.96 | 0.75 |
| | Cladosporium cladosporioides TAMU565 (MIC-20835) | 44.70 ± 6.76 | 0.60 |
| | Cladosporium cladosporioides TAMU517 (MIC-72531) | 44.65 ± 4.36 | 0.60 |
| | TAMU508 (MIC-72092) | 44.60 ± 4.17 | 0.60 |
| | Fusarium sp. TAMU340 (MIC-87502) | 43.40 ± 5.62 | 0.48 |
| | Chaetomium globosum (TAMU353) | 42.75 ± 4.36 | 0.42 |
| | Penicillium sp. TAMU413 (MIC-50324) | 40.24 ± 5.52 | 0.24 |
| | Cladosporium sp. TAMU501 (MIC-31246) | 34.34 ± 3.46 | 0.038 |
| | Purpureocillium lavendulum TAMU424 (MIC-21610) | 33.60 ± 5.41 | 0.029 |
| Round 7 | Control | 44.49 ± 3.88 | — |
| | Chaetomium globosum TAMU559 (MIC-22947) | 34.86 ± 2.08 | 0.056 |
| | Epicoccum nigrum TAMU488 (MIC-85153) | 33.86 ± 1.93 | 0.026 |
| | Epicoccum nigrum TAMU497 (MIC-90504) | 30.26 ± 2.28 | 0.0008 |
| | Cladosporium gossypiicola TAMU249 (MIC-80602) | 28.69 ± 2.54 | 0.0001 |
| | Epicoccum nigrum TAMU100 (MIC-77047) | 22.49 ± 2.59 | <0.0001 |
| | Epicoccum nigrum TAMU194 (MIC-76091) | 22.35 ± 2.63 | <0.0001 |
| | Diaporthe sp. TAMU137 (MIC-42067) | 18.82 ± 2.70 | <0.0001 |
| | Cladosporium sp. TAMU463 (MIC-91557) | 16.27 ± 2.05 | <0.0001 |
| | Epicoccum nigrum TAMU128 (MIC-65047) | 13.85 ± 1.95 | <0.0001 |
| | Chaetomium globosum TAMU560 (MIC-44512) | 12.79 ± 2.89 | <0.0001 |
| | Alternaria eichhorniae. TAMU452 (MIC-26235) | 12.55 ± 1.62 | <0.0001 |
| Round 8 | Control | 47.03 ± 3.57 | — |
| | Cladosporium sp. TAMU501 (MIC-31246) | 31.37 ± 2.70 | <0.0001 |
| | Epicoccum nigrum TAMU125 (MIC-51347) | 27.00 ± 2.16 | <0.0001 |
| | Epicoccum nigrum TAMU131 (MIC-85590) | 22.25 ± 2.43 | <0.0001 |
| | Purpureocillium lavendulum TAMU424 (MIC-21610) | 22.03 ± 3.14 | <0.0001 |
| | Cladosporium cladosporioides TAMU474 (MIC-34220) | 19.41 ± 1.83 | <0.0001 |
| | Alternaria sp. TAMU53 (MIC-34397) | 18.66 ± 2.39 | <0.0001 |
| | Chaetomium sp. TAMU87 (MIC-78639) | 17.60 ± 1.94 | <0.0001 |
| | Purpureocillium lilacinum TAMU239 (MIC-86415) | 15.45 ± 1.64 | <0.0001 |
| | Chaetomium sp. TAMU110 (MIC-66827) | 14.44 ± 1.31 | <0.0001 |
| | Chaetomium coarctatum TAMU333 (MIC-39959) | 14.40 ± 1.55 | <0.0001 |
| | Chaetomium globosum TAMU554 (MIC-33414) | 14.14 ± 1.57 | <0.0001 |

TABLE 7
The effects of 56 endophytic fungal isolates from cotton on
root-knot nematode gall production summarized by genera.
Increased resistance was defined as a statistically significant
decrease in the number of galls produced by root-knot nematode
per gram of root tissue compared to the corresponding control
treatment. Decreased resistance was defined as a statistically
significant increase in the number of galls produced by root-knot
nematode per gram of root tissue compared to the corresponding
control treatment. Where an endophyte treatement was tested in
more than one round of the experiment, increased or decreased
resitance was counted in this table only if results were
consistent in direction and significance between the rounds.

| Fungal | Increased | | Decreased | |
|---|---|---|---|---|
| Taxonomic ID | Resistance | No effect | Resistance | Sub-Total |
| Acremonium | 0 | 1 | 0 | 1 |
| Alternaria | 2 | 2 | 2 | 6 |
| Beauveria | 0 | 1 | 0 | 1 |
| Chaetomium | 5 | 6 | 0 | 11 |
| Cladosporium | 4 | 10 | 0 | 14 |
| Cryptococcus | 0 | 1 | 0 | 1 |
| Curvularia | 0 | 1 | 1 | 2 |
| Diaporthe | 1 | 0 | 0 | 1 |
| Didymellaceae | 0 | 1 | 0 | 1 |
| Epicoccum | 7 | 5 | 0 | 12 |
| Fusarium | 0 | 1 | 0 | 1 |
| Nectriaceae | 0 | 1 | 0 | 1 |
| Penicillium | 0 | 1 | 0 | 1 |
| Purpureocillium | 2 | 0 | 0 | 2 |
| Unknown | 0 | 1 | 0 | 1 |
| Total | 21 | 32 | 3 | 56 |

### Example 8: Fungal Endophytes Reduce Nematode Reproduction

Plants are germinated from treated and untreated control seeds in an environment chamber and then transplanted to soil in pots 11 days after planting. Two replicate seedlings per treatment are sampled to examine the endophyte colonization efficiency by surface sterilization and plating on PDA agar. Nematode treatment group seedlings are treated with either 2,000 or 10,000 eggs/plant at day six after transplanting. Plants are harvested and processed 6 weeks after nematode inoculation. The numbers of galls per gram of root tissue and total egg numbers in the population for each plant are quantified to compare nematode performance between endophyte-treated and untreated (control) plants.

### Example 9: Herbivory Assays: Soy and Cabbage Looper on Soybean Plants

Endophyte treatments and untreated controls (no endophyte) were prepared as described in Example 3. Thirty-six fungal endophyte isolates were screened with the detached leaf herbivory assay.

Plant management: Two soybean seeds were planted in each 4 cm top diameter×6 cm deep pot, with 15 pots for each treatment. Potting media consists of bark, vermiculite, peat moss, perlite, and dolomitic limestone (non-sterile Metro-Mix® 900 soil, Sun Gro Horticulture, Agawam, MA). Soybean seedlings in individual pot were thinned to one plant per plot after the unifoliate leaves have unfolded. Plants were caged and maintained in the greenhouse.

Detached leaf assay: The first trifoliate leaves were collected from each soybean plant when fully expanded. The two lateral leaflets were separated and distributed to 1.5% agar plates for insect infestation, with one leaflet per Petri dish per insect species; soybean looper (Chrysodeixis includens) and cabbage looper (Trichoplusia ni). Both the plants and plates were labeled to ensure the insects received leaf tissues from the same plant throughout each assay. The eggs of both soybean loopers and cabbage loopers were hatched in zipper bags in an incubating room (under 25±3° C. with 12 hours light: 12 hours dark). Three neonates of each species were transferred onto each 1.5% agar plate with one piece of dissected leaflet. Petri dishes were sealed and stored in a Thermo incubator at 27.5±0.5° C.

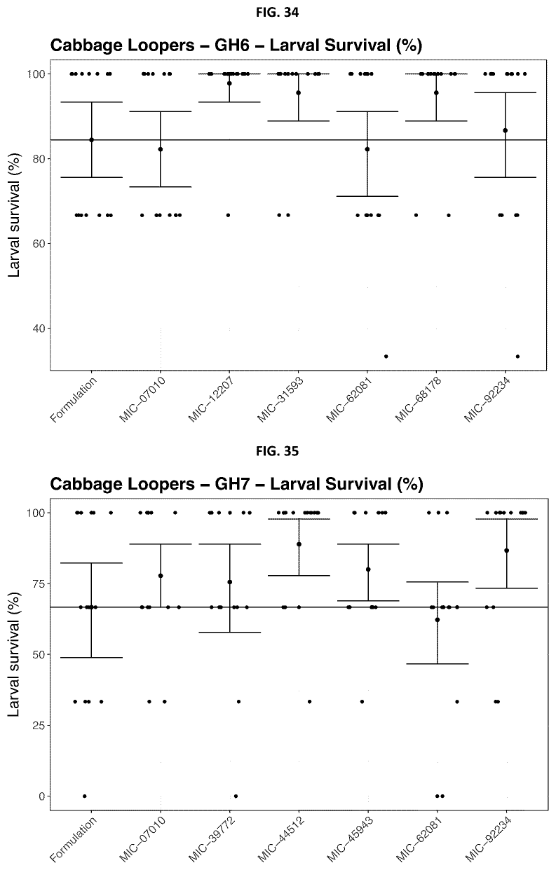

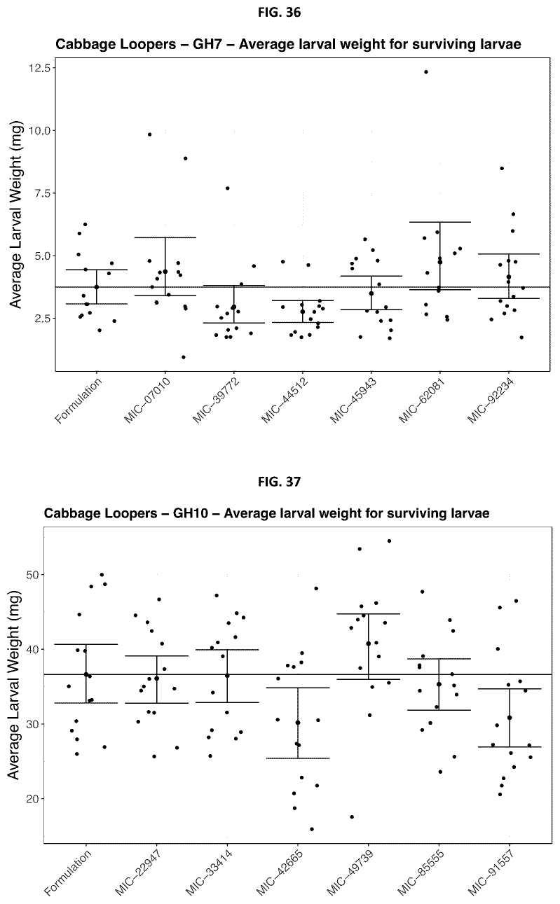

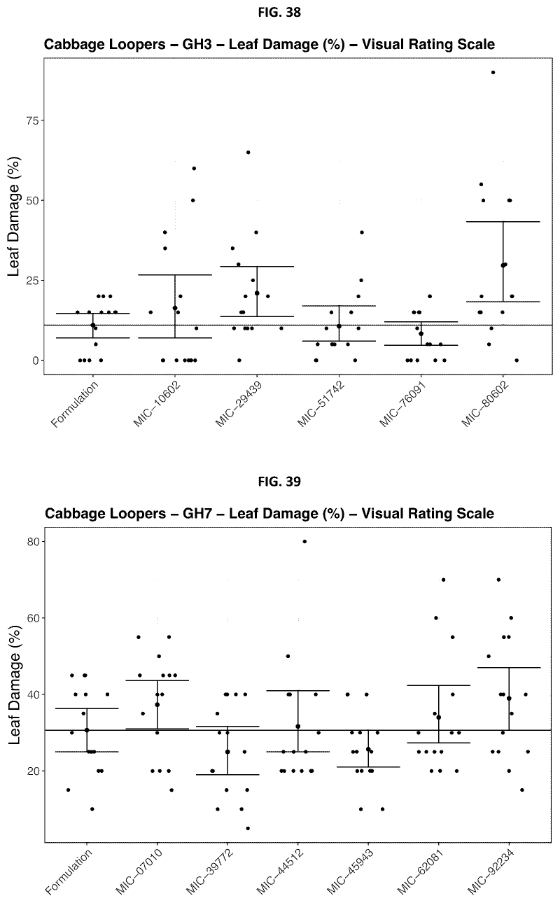

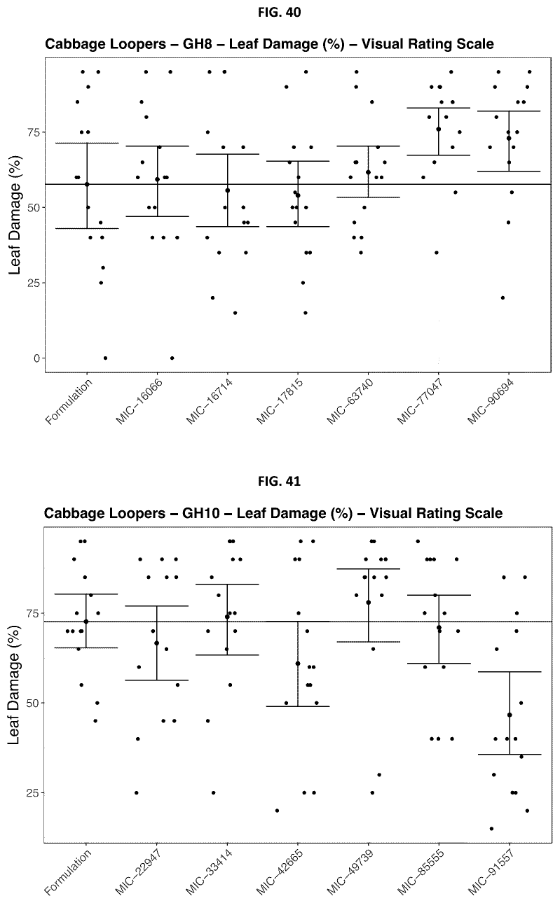

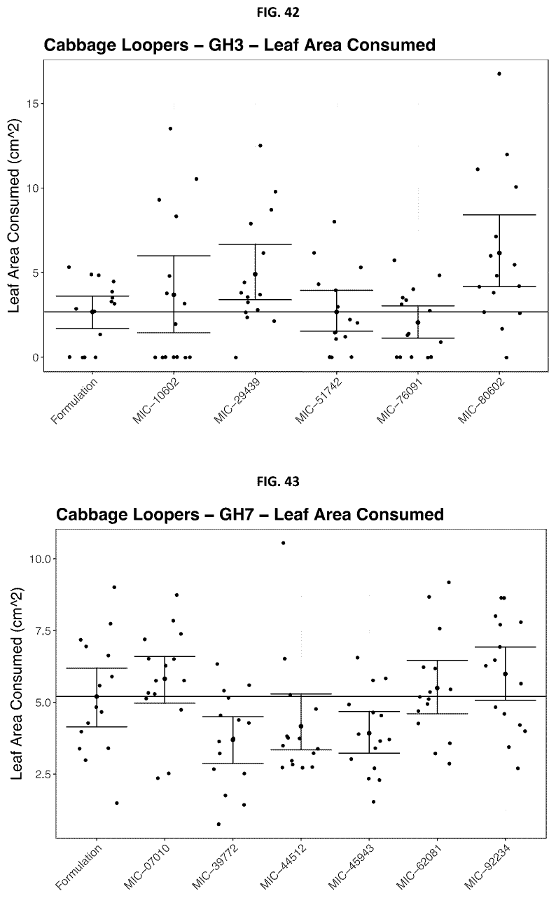

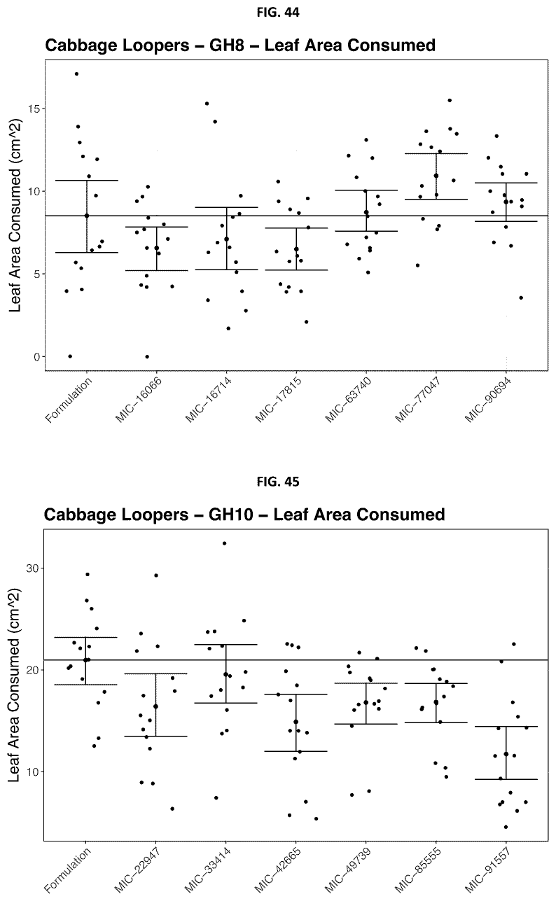

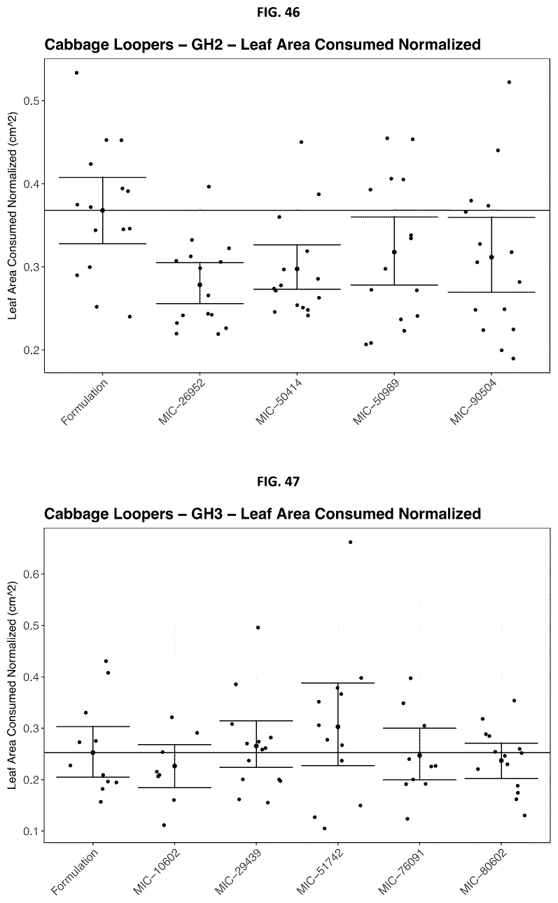

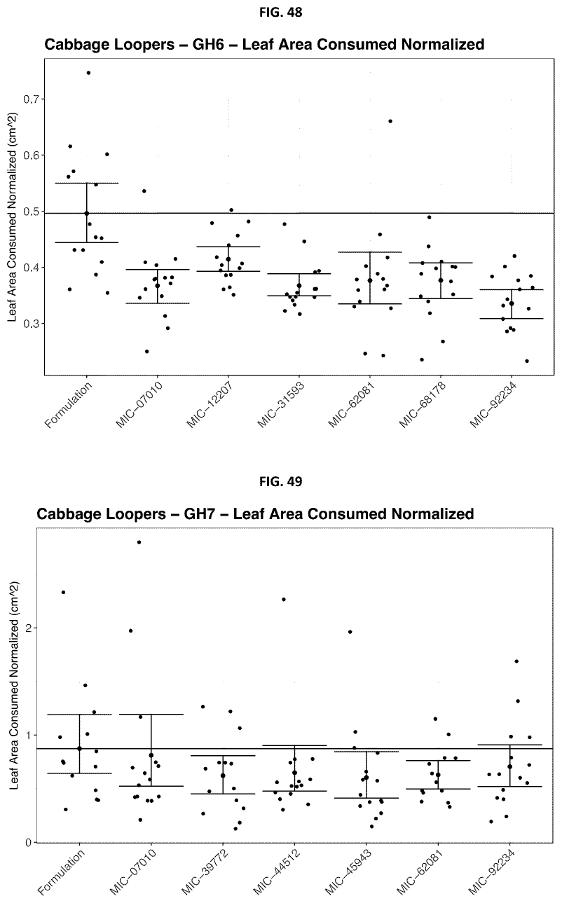

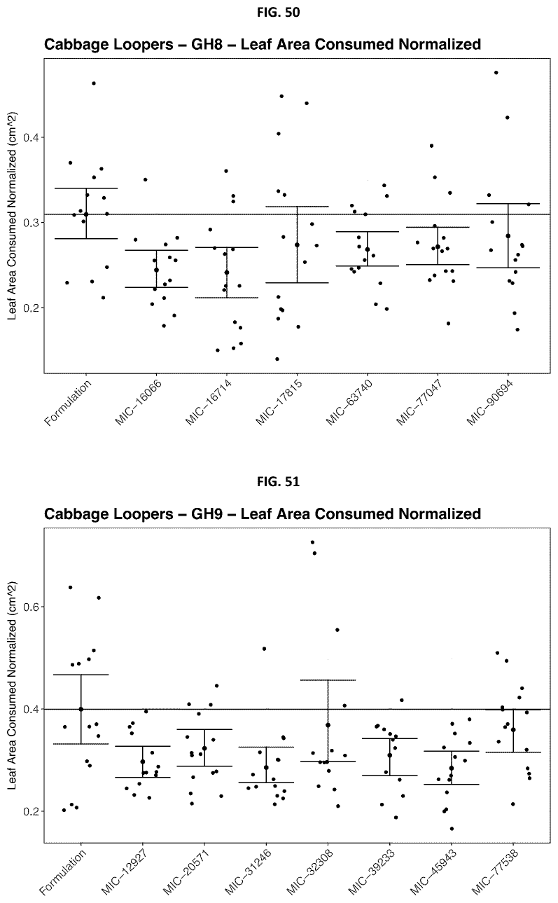

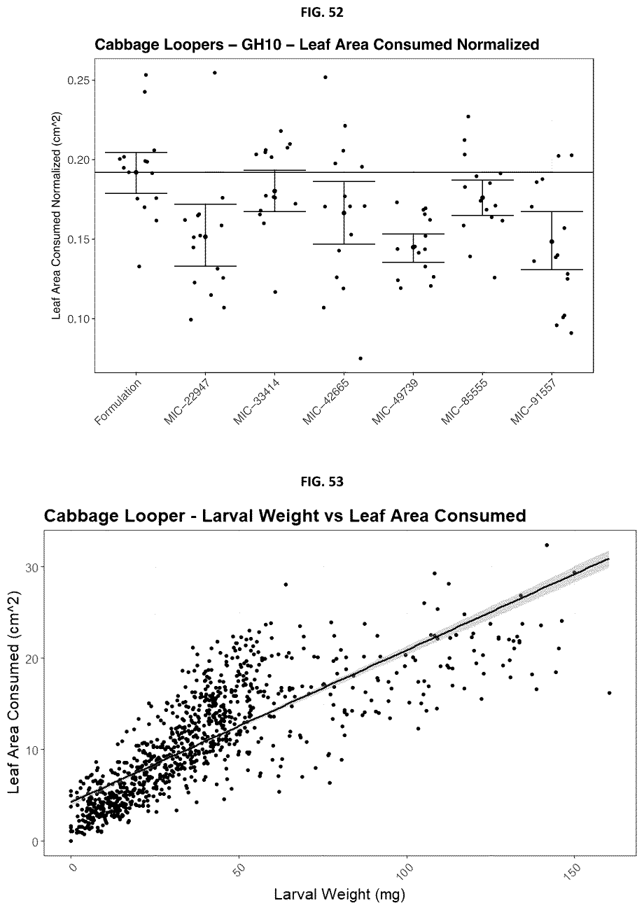

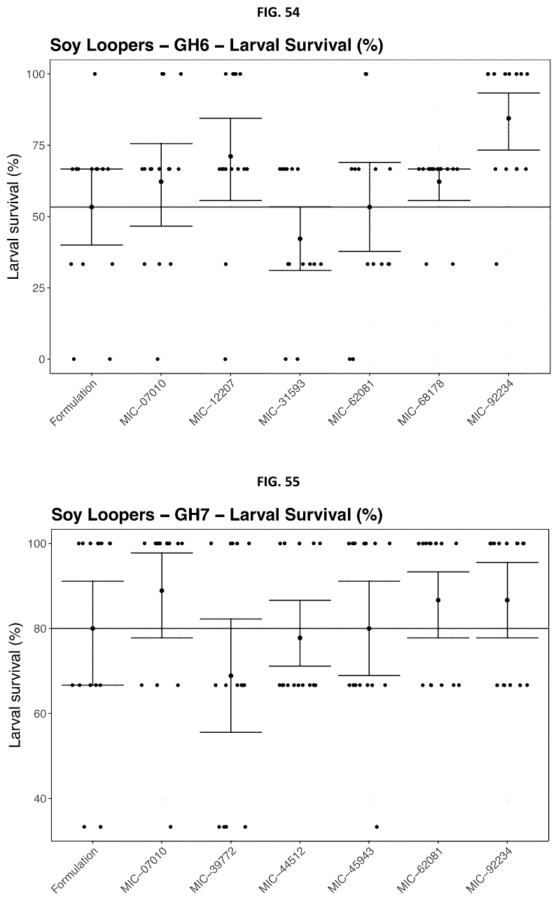

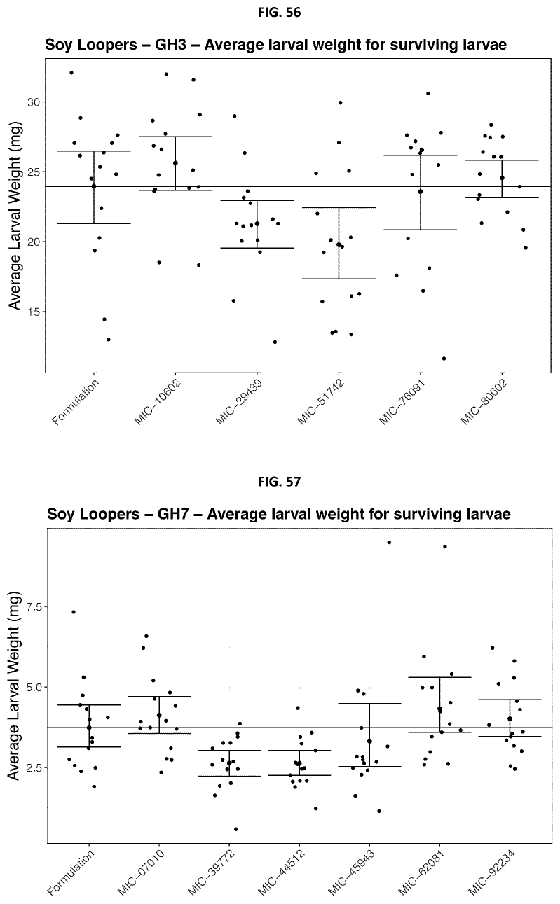

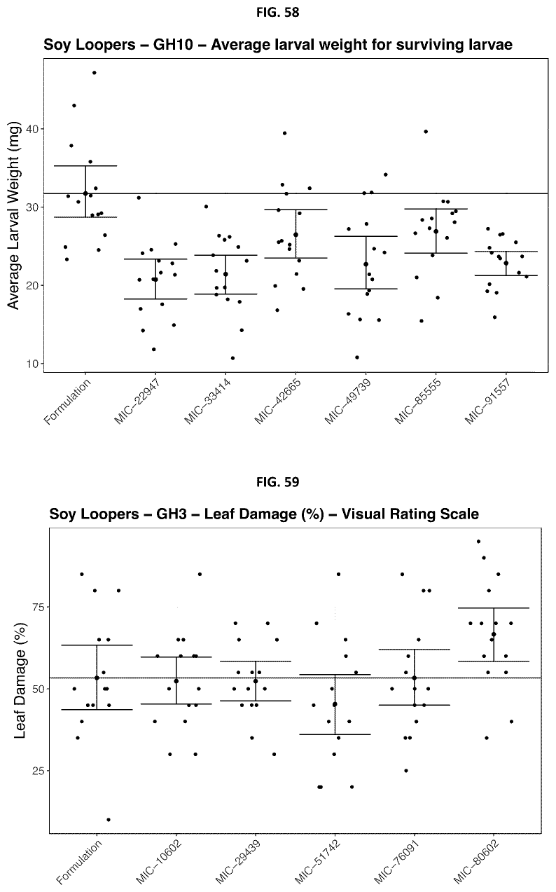

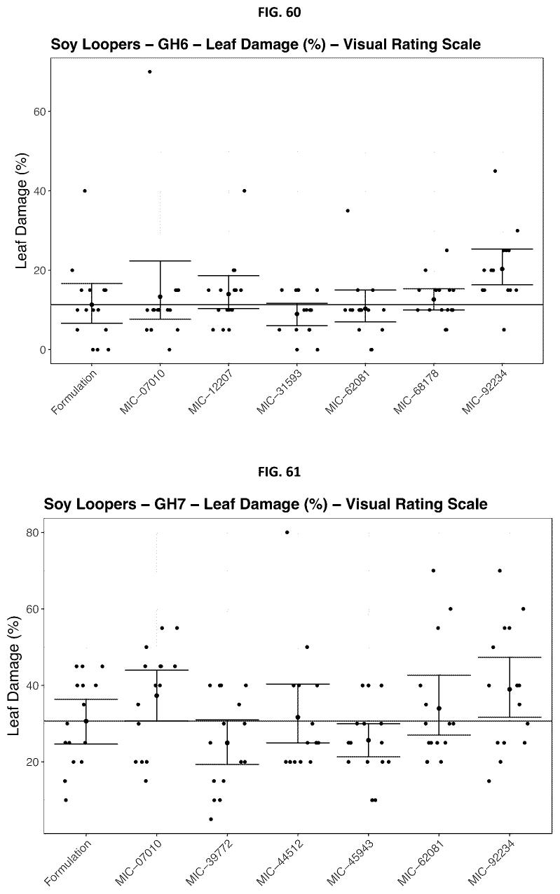

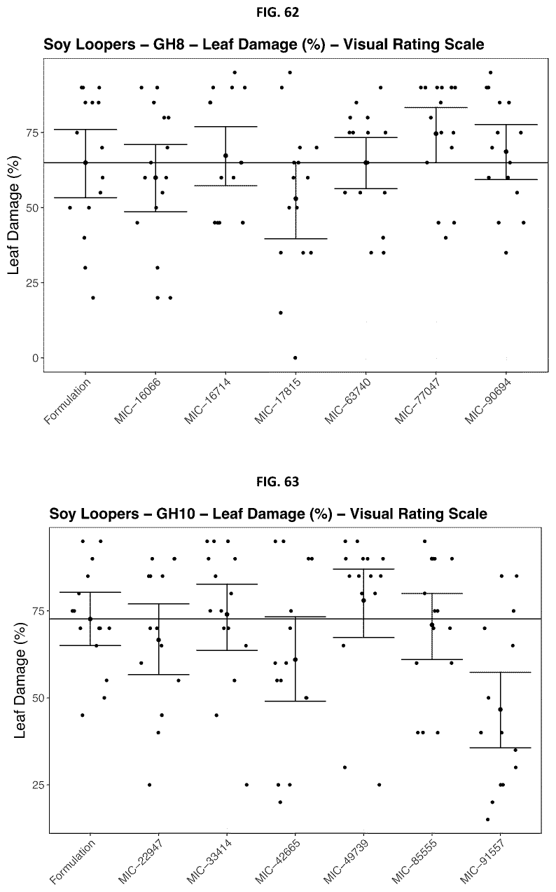

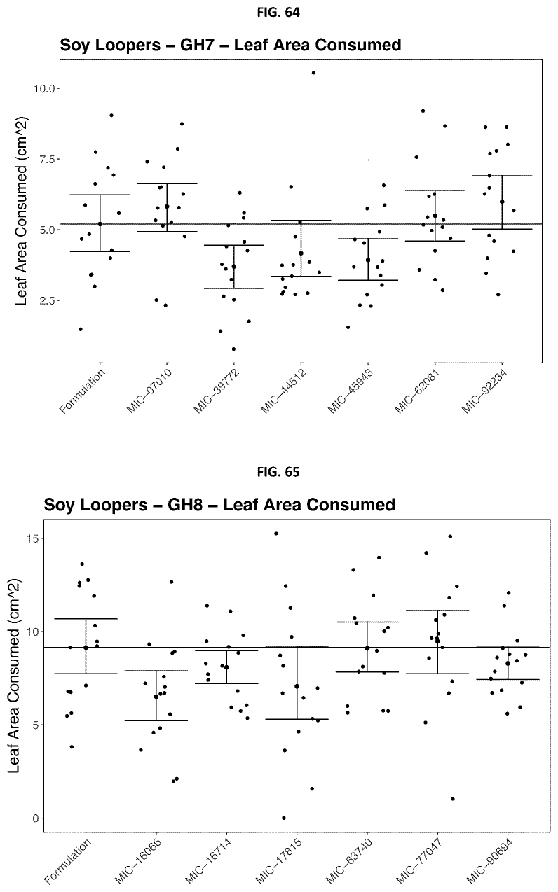

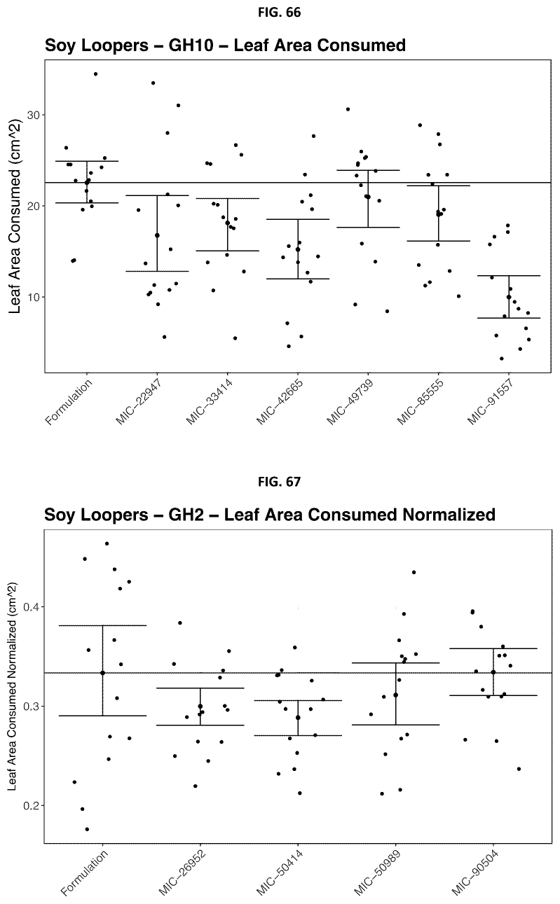

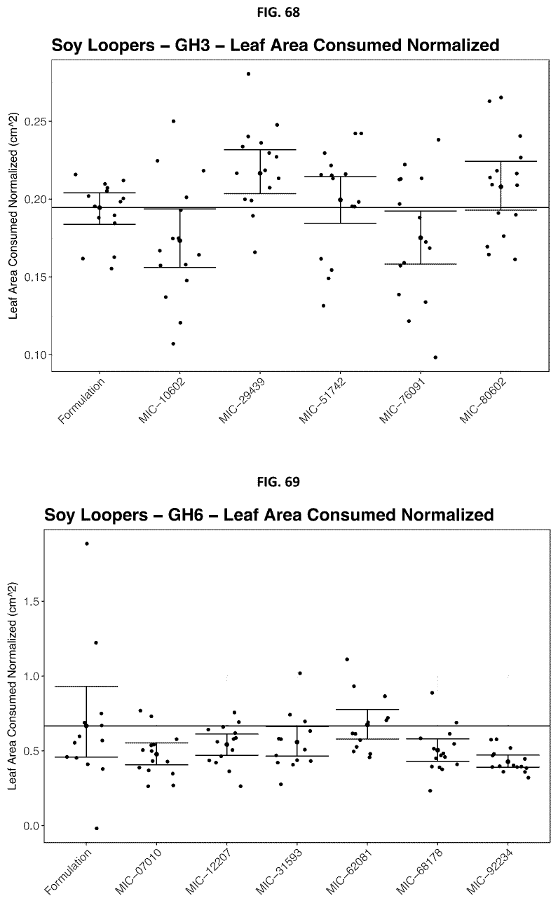

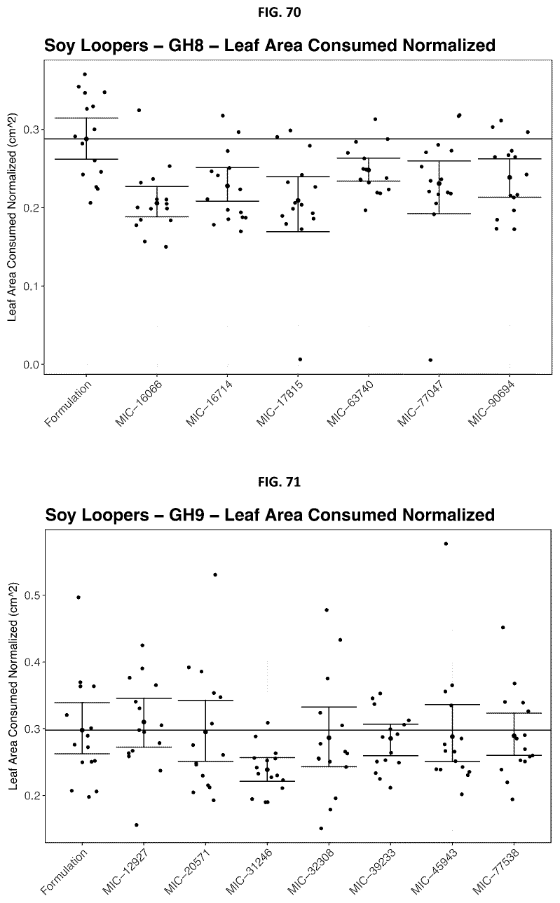

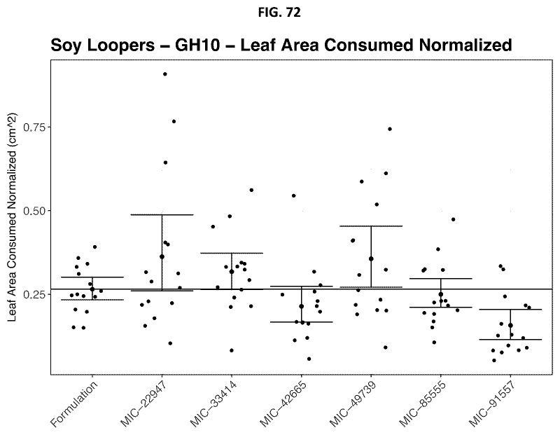

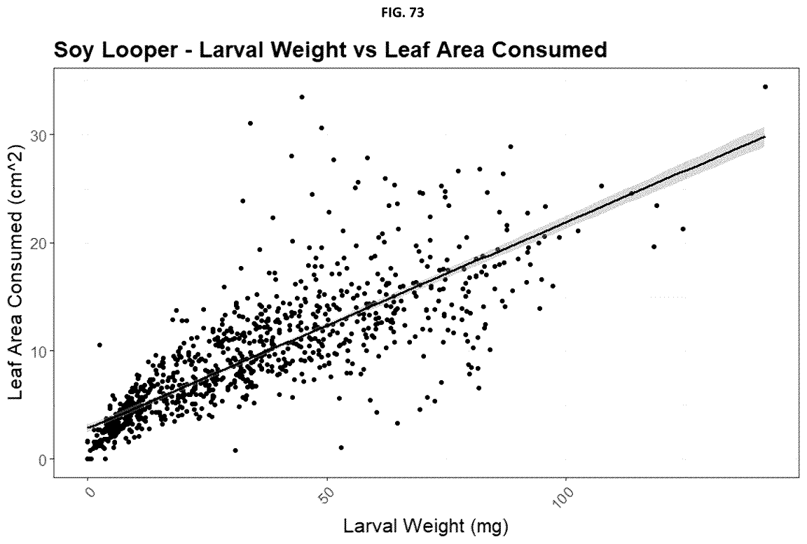

Five days after the initial set up, the old leaf tissues were replaced by one lateral leaflet of the second fully expanded trifoliate leave per Petri dish. All leaf tissues were freshly obtained from the soybean plants described above. Petri dishes were sealed and stored in a Thermo incubator at 27.5±0.5° C. for two days. To terminate the assay, the remaining leaf tissues were removed from each Petri dish and placed on a log sheet for image collections. The percentage of leaf area consumed (FIGS. 42-45 and FIGS. 64-66) was estimated using a soybean leaf defoliation chart as the reference (Ortega et al., Pyramids of QTLs enhance host-plant resistance and Bt-mediated resistance to leaf-chewing insects in soybean. Theor Appl Genet. 2016 April; 129(4):703-715). Whole leaf area (cm2) was calculated in Image J (Abramoff, M. D., Magalhaes, P. J., Ram, S. J. “Image Processing with ImageJ”. Biophotonics International, volume 11, issue 7, pp. 36-42, 2004). Larval survivorship was recorded for each Petri dish (FIGS. 34-35 and FIGS. 54-55). Larval size was recorded as the total larval weight (mg) of all survivors from each Petri dish (FIGS. 36-37, FIGS. 56-58). The percentage leaf area was normalized for larval weight (FIGS. 46-52 and FIGS. 67-72). Larval weight and leaf area consumed for the whole data set was compiled (FIG. 53).

Each round of the experiment (indicated by trial id) contained internal controls. For cabbage looper formulation treated controls, larval survival ranged from roughly 70-100% with poor survival for a single round (GH3-30%), larval weight ranged from roughly 8-15 mg with two rounds showing extreme results (GH7-3 mg, GH10-37 mg), leaf area consumed ranged from roughly 5-11 cm\hat{ }2 with two rounds showing extreme results (GH3-3 cm\hat{ }2, GH10-21 cm\hat{ }2). For soy looper formulation treated controls, larval survival ranged from roughly 80-100% with poor survival for a single round (GH6-53%), larval weight ranged from roughly 11-23 mg with three rounds showing extreme results (GH6-2.5 mg, GH7-4 mg, GH10-33 mg), leaf area consumed ranged from roughly 5-13 cm\hat{ }2 with two rounds showing extreme results (GH6-3 cm\hat{ }2, GH10-22 cm\hat{ }2).

In the cabbage looper assays, fifteen isolates reduced caterpillar defoliation by 15% or more compared to controls, twelve of which also reduced larval growth compared to controls. All fifteen isolates reduced defoliation standardized by larval weight compared to controls. In the soybean looper assays, ten fungal isolates reduced caterpillar defoliation by 15% (Table 10) or more as well as reducing larval growth (Tables 8). When comparing performance across insect species, 22 (77%) of the fungal isolates showed the same trend, either positive or negative, in impact on leaf area consumed. Only 8 fungal isolates (22%) showed a variable response across the two insect species with a reduction in leaf area consumed for one insect and an increase in leaf area consumed for the other (Table 10).

For fungal taxa with multiple isolates included in the experiment, it was possible to compare performance across isolates. In the case of Bipolaris spicifera, two isolates (TAM00189 and TAM00013) consistently increased herbivory as measured by increased larval weight and leaf area consumption while one isolate (TAM00439) had a variable response. For Chaetomium globosum, all isolates showed a decrease in herbivory with two isolates (TAM00560 and TAM559) showing a decrease in leaf area consumption of well over 15% for both cabbage and soy loopers (Table 10). For Cladosporium herbarum, 5 isolates showed an increase in herbivory with 3 isolates showing over an 80% increase in leaf area consumed for cabbage looper compared to formulation controls (Table 10). One C. herbarum isolate showed a variable response in leaf area consumption across insect species and two isolates (TAM00494 and TAM00463) consistently decreased herbivory with one of the isolates (TAM00463) showing a decrease in herbivory over 40% in both insect species as the top performer in the experiment as a whole (Table 10). In the case of Gibellulopsis nigrescens, all three isolates (TAM00531, TAM00533 and TAM00524) decreased herbivory across both insects (Table 10). And for Epicoccum nigrum, 2 isolates (TAM00074 and TAM00536) showed consistent reduction in herbivory across both insects, 2 showed a variable response in each insect and 3 isolates consistently increased herbivory across both insects.

TABLE 8
Effect of fungal endophyte treatment on cabbage and soybean looper larvae weight in soybean
detached leaf assay.

| | | | | | Soybean Looper |
|---|---|---|---|---|---|
| | | | | Cabbage Looper | (Chrysodeixis |
| | | | | (Trichoplusia ni) | includens) |
| | | | | % Change in | % Change in |
| | | | | Average Larval | Average Larval |
| Round | TAMID | TAXID | MICID | Weight | Weight |
| GH9 | TAM00323 | Alternaria planifunda | MIC-39233 | −43.1 | 23.9 |
| GH6 | TAM00189 | Curvularia spicifera | MIC-31593 | 140.3 | 12.6 |
| GH7 | TAM00013 | Bipolaris spicifera | MIC-92234 | 42.8 | 17.5 |
| GH9 | TAM00439 | Bipolaris spicifera | MIC-77538 | −39.5 | 4.4 |
| GH10 | TAM00554 | Chaetomium globosum | MIC-33414 | −2.1 | −33.0 |
| GH10 | TAM00559 | Chaetomium globosum | MIC-22947 | −3.4 | −43.2 |
| GH7 | TAM00560 | Chaetomium globosum | MIC-44512 | −3.8 | −29.4 |
| GH7 | TAM00317 | Chaetomium sp. | MIC-39772 | −8.1 | −35.2 |
| GH9 | TAM00501 | Cladosporium sp. | MIC-31246 | 126.6 | 84.7 |
| GH3 | TAM00249 | Cladosporium gossypiicola | MIC-80602 | 82.0 | 5.8 |
| GH3 | TAM00201 | Cladosporium sp. | MIC-29439 | 66.5 | −8.1 |
| GH3 | TAM00248 | Cladosporium sp. | MIC-10602 | 38.4 | 7.1 |
| GH9 | TAM00193 | Cladosporium cladosporioides | MIC-12927 | 32.9 | 8.6 |
| GH2 | TAM00534 | Cladosporium oxysporum | MIC-50414 | 30.6 | 13.6 |
| GH2 | TAM00494 | Cladosporium sp. | MIC-26952 | 19.3 | 2.9 |
| GH10 | TAM00463 | Cladosporium sp. | MIC-91557 | −30.4 | −24.6 |
| GH6 | TAM00105 | Curvularia protuberata | MIC-07010 | 76.9 | 49.5 |
| GH7 | TAM00105 | Curvularia protuberata | MIC-07010 | 29.6 | 21.9 |
| GH6 | TAM00032 | Epicoccum nigrum | MIC-68178 | 107.0 | 43.1 |
| GH8 | TAM00100 | Epicoccum nigrum | MIC-77047 | 41.8 | 23.4 |
| GH8 | TAM00046 | Didymellaceae | MIC-90694 | 22.3 | 6.2 |
| GH7 | TAM00103 | Epicoccum nigrum | MIC-62081 | 15.5 | 21.5 |
| GH8 | TAM00536 | Epicoccum nigrum | MIC-16066 | −2.4 | −3.3 |
| GH2 | TAM00497 | Epicoccum nigrum | MIC-90504 | −3.0 | 6.7 |
| GH10 | TAM00074 | Epicoccum nigrum | MIC-85555 | −12.8 | −11.1 |
| GH8 | TAM00531 | Gibellulopsis nigrescens | MIC-16714 | 15.5 | 10.2 |
| GH10 | TAM00533 | Gibellulopsis nigrescens | MIC-49739 | 6.1 | −24.6 |
| GH10 | TAM00524 | Gibellulopsis nigrescens | MIC-42665 | −20.0 | −16.3 |
| GH3 | TAM00251 | Chaetomium piluliferum | MIC-51742 | −15.8 | −17.0 |
| GH2 | TAM00490 | Phialemonium inflatum | MIC-50989 | 34.6 | 7.2 |
| GH3 | TAM00194 | Epicoccum nigrum | MIC-76091 | −18.1 | 4.0 |
| GH6 | TAM00296 | Pleosporaceae sp. | MIC-12207 | 126.1 | 57.0 |
| GH8 | TAM00518 | Preussia africana | MIC-17815 | −8.0 | −6.9 |
| GH8 | TAM00504 | Sphaerulina pseudovirgaureae | MIC-63740 | 14.3 | 9.2 |
| GH9 | TAM00160 | Stagonospora sp. | MIC-20571 | 72.1 | 35.3 |
| GH9 | TAM00473 | Stemphylium herbarum | MIC-32308 | 19.4 | 0.9 |
| GH9 | TAM00362 | Stemphylium herbarum | MIC-45943 | 7.2 | 29.6 |

TABLE 9
Effect of fungal endophyte treatment on cabbage and soybean
looper larvae survival in soybean detached leaf assay.

| | | | | Cabbage Looper | Soybean Looper |
|---|---|---|---|---|---|
| | | | | (Trichoplusia ni) | (Chrysodeixis includens) |
| | | | | % Change in | % Change in |
| | | | | Surviving | Surviving |
| Round | TAMID | TAXID | MICID | Larvae Percent | Larvae Percent |
| GH9 | TAM00323 | Alternaria planifunda | MIC-39233 | −3.45 | 4.65 |
| GH7 | TAM00013 | Bipolaris spicifera | MIC-92234 | 30 | 8.33 |
| GH6 | TAM00189 | Curvularia spicifera | MIC-31593 | 13.16 | −20.83 |
| GH9 | TAM00439 | Bipolaris spicifera | MIC-77538 | 3.45 | 2.33 |
| GH7 | TAM00560 | Chaetomium globosum | MIC-44512 | 33.33 | −2.78 |
| GH10 | TAM00554 | Chaetomium globosum | MIC-33414 | −2.22 | 0 |
| GH10 | TAM00559 | Chaetomium globosum | MIC-22947 | −2.22 | −11.9 |
| GH7 | TAM00317 | Chaetomium sp. | MIC-39772 | 13.33 | −13.89 |
| GH3 | TAM00201 | Cladosporium sp. | MIC-29439 | 53.33 | 4.76 |
| GH9 | TAM00501 | Cladosporium sp. | MIC-31246 | 34.48 | 0 |
| GH9 | TAM00193 | Cladosporium cladosporioides | MIC-12927 | 20.69 | 0 |
| GH3 | TAM00249 | Cladosporium gossypiicola | MIC-80602 | 20 | 4.76 |
| GH2 | TAM00494 | Cladosporium sp. | MIC-26952 | 10 | 4.76 |
| GH3 | TAM00248 | Cladosporium sp. | MIC-10602 | 6.67 | 2.38 |
| GH2 | TAM00534 | Cladosporium oxysporum | MIC-50414 | 2.5 | 4.76 |
| GH10 | TAM00463 | Cladosporium sp. | MIC-91557 | −13.33 | 4.76 |
| GH7 | TAM00105 | Curvularia protuberata | MIC-07010 | 16.67 | 11.11 |
| GH6 | TAM00105 | Curvularia protuberata | MIC-07010 | −2.63 | 16.67 |
| GH8 | TAM00100 | Epicoccum nigrum | MIC-77047 | 36.67 | −4.65 |
| GH8 | TAM00046 | Didymellaceae | MIC-90694 | 23.33 | −2.33 |
| GH8 | TAM00536 | Epicoccum nigrum | MIC-16066 | 16.67 | −4.65 |
| GH6 | TAM00032 | Epicoccum nigrum | MIC-68178 | 13.16 | 16.67 |
| GH2 | TAM00497 | Epicoccum nigrum | MIC-90504 | 7.5 | 4.76 |
| GH7 | TAM00103 | Epicoccum nigrum | MIC-62081 | −6.67 | 8.33 |
| GH10 | TAM00074 | Epicoccum nigrum | MIC-85555 | −8.89 | 4.76 |
| GH8 | TAM00531 | Gibellulopsis nigrescens | MIC-16714 | 23.33 | −6.98 |
| GH10 | TAM00524 | Gibellulopsis nigrescens | MIC-42665 | −2.22 | 0 |
| GH10 | TAM00533 | Gibellulopsis nigrescens | MIC-49739 | −4.44 | 4.76 |
| GH3 | TAM00251 | Chaetomium piluliferum | MIC-51742 | 20 | 2.38 |
| GH2 | TAM00490 | Phialemonium inflatum | MIC-50989 | 10 | 4.76 |
| GH3 | TAM00194 | Epicoccum nigrum | MIC-76091 | 20 | 7.14 |
| GH6 | TAM00296 | Pleosporaceae sp. | MIC-12207 | 15.79 | 33.33 |
| GH8 | TAM00518 | Preussia africana | MIC-17815 | 30 | −6.98 |
| GH8 | TAM00504 | Sphaerulina pseudovirgaureae | MIC-63740 | 36.67 | 4.65 |
| GH9 | TAM00160 | Stagonospora sp. | MIC-20571 | 41.38 | 4.65 |
| GH9 | TAM00362 | Stemphylium herbarum | MIC-45943 | 24.14 | −4.65 |
| GH9 | TAM00473 | Stemphylium herb arum | MIC-32308 | 17.24 | −9.3 |

TABLE 10
Effect of fungal endophyte treatment on cabbage and soybean looper larvae leaf area consumption
in soybean detached leaf assay.

| | | | | Cabbage Looper | Soybean Looper |
|---|---|---|---|---|---|
| | | | | (Trichoplusia ni) | (Chrysodeixis |
| | | | | % Change in | includens) |
| | | | | Leaf Area | % Change in Leaf |
| Round | TAMID | TAXID | MICID | Consumed | Area Consumed |
| GH9 | TAM00323 | Alternaria planifunda | MIC-39233 | −48.9 | 18.87 |
| GH6 | TAM00189 | Curvularia spicifera | MIC-31593 | 81.24 | 1.83 |
| GH7 | TAM00013 | Bipolaris spicifera | MIC-92234 | 15.14 | 15.14 |
| GH9 | TAM00439 | Bipolaris spicifera | MIC-77538 | −39.22 | 4.79 |
| GH10 | TAM00554 | Chaetomium globosum | MIC-33414 | −6.71 | −19.62 |
| GH7 | TAM00560 | Chaetomium globosum | MIC-44512 | −19.85 | −19.85 |
| GH10 | TAM00559 | Chaetomium globosum | MIC-22947 | −21.73 | −25.66 |
| GH7 | TAM00317 | Chaetomium sp. | MIC-39772 | −28.92 | −28.92 |
| GH9 | TAM00501 | Cladosporium sp. | MIC-31246 | 86.48 | 42.88 |
| GH3 | TAM00201 | Cladosporium sp. | MIC-29439 | 82.67 | 1.91 |
| GH3 | TAM00249 | Cladosporium | MIC-80602 | 80 | 12.44 |
| | | gossypiicola | | | |
| GH3 | TAM00248 | Cladosporium sp. | MIC-10602 | 37.3 | −5.49 |
| GH9 | TAM00193 | Cladosporium | MIC-12927 | 16.22 | 11.88 |
| | | cladosporioides | | | |
| GH2 | TAM00534 | Cladosporium | MIC-50414 | 5.91 | 4.71 |
| | | oxysporum | | | |
| GH2 | TAM00494 | Cladosporium sp. | MIC-26952 | −9.13 | −4.49 |
| GH10 | TAM00463 | Cladosporium sp. | MIC-91557 | −43.98 | −55.69 |
| GH6 | TAM00105 | Curvularia protuberata | MIC-07010 | 32.63 | 15.28 |
| GH7 | TAM00105 | Curvularia protuberata | MIC-07010 | 11.9 | 11.9 |
| GH6 | TAM00032 | Epicoccum nigrum | MIC-68178 | 63.45 | 10.16 |
| GH8 | TAM00100 | Epicoccum nigrum | MIC-77047 | 28.42 | 3.69 |
| GH8 | TAM00046 | Didymellaceae | MIC-90694 | 9.85 | −9.27 |
| GH7 | TAM00103 | Epicoccum nigrum | MIC-62081 | 5.72 | 5.72 |
| GH2 | TAM00497 | Epicoccum nigrum | MIC-90504 | −18.92 | 13.48 |
| GH10 | TAM00074 | Epicoccum nigrum | MIC-85555 | −19.67 | −15.5 |
| GH8 | TAM00536 | Epicoccum nigrum | MIC-16066 | −22.85 | −28.8 |
| GH8 | TAM00531 | Gibellulopsis nigrescens | MIC-16714 | −16.55 | −11.6 |
| GH10 | TAM00533 | Gibellulopsis nigrescens | MIC-49739 | −19.85 | −6.98 |
| GH10 | TAM00524 | Gibellulopsis nigrescens | MIC-42665 | −28.9 | −32.53 |
| GH3 | TAM00251 | Chaetomium piluliferum | MIC-51742 | −0.26 | −13.41 |
| GH2 | TAM00490 | Phialemonium inflatum | MIC-50989 | 11.69 | 3.91 |
| GH3 | TAM00194 | Epicoccum nigrum | MIC-76091 | −23.32 | −6.34 |
| GH6 | TAM00296 | Pleosporaceae sp. | MIC-12207 | 93.94 | 41.02 |
| GH8 | TAM00518 | Preussia africana | MIC-17815 | −23.68 | −22.66 |
| GH8 | TAM00504 | Sphaerulina | MIC-63740 | 2.51 | −0.41 |
| | | pseudovirgaureae | | | |
| GH9 | TAM00160 | Stagonospora sp. | MIC-20571 | 62.35 | 32.82 |
| GH9 | TAM00473 | Stemphylium herbarum | MIC-32308 | 10.58 | −4.74 |
| GH9 | TAM00362 | Stemphylium herbarum | MIC-45943 | −10.54 | 17.71 |

### Example 10: Treatment of Aphid Infestation

Seed treatment: Fungal endophyte biomass was prepared and heterologously disposed on black cotton seeds of varieties Phytogen 499WRF and Delta Pine 1321B2RF as described in Example 3.

Plant production: Seeds of each treatment combination were planted individually in seedling germination trays. Each cell pot measured 4 cm top diameter×6 cm deep and was filled with nonsterile Metro-Mix® 900 soil (Sun Gro Horticulture, Agawam, MA; ingredients: bark, vermiculite, peat moss, perlite, dolomitic limestone) watered to saturation prior to planting. Plants were grown in a controlled temperature room at 25° C. under constant overhead illumination (EnviroGro T5 High Output Fluorescent Lighting Systems).

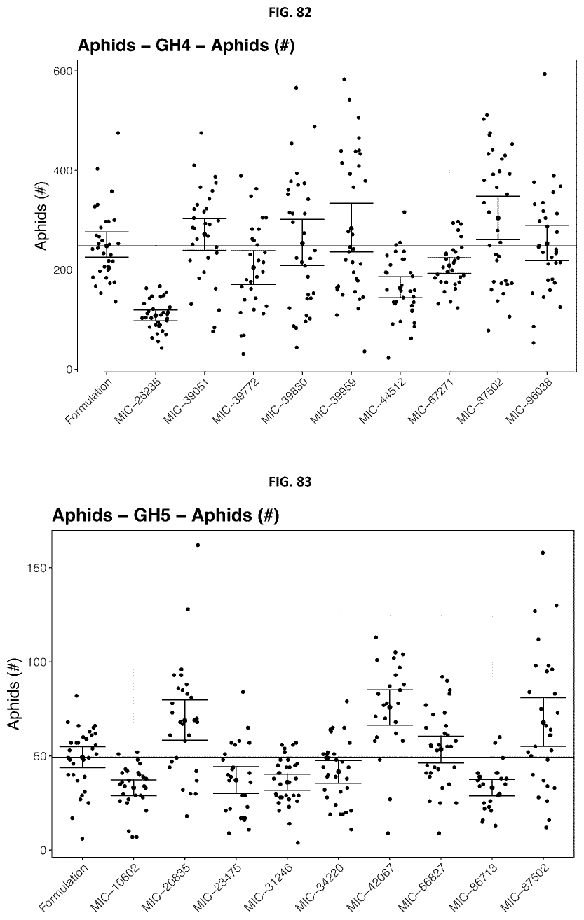

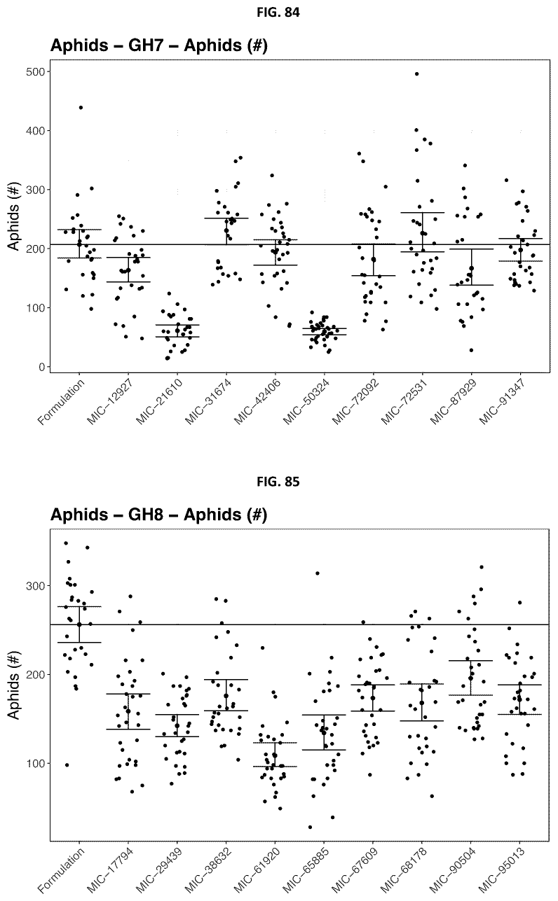

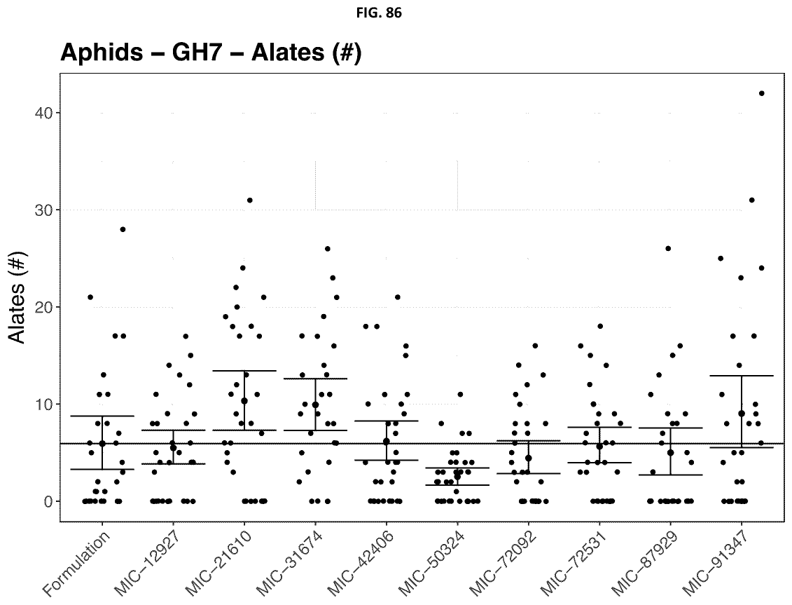

Aphid infestation: Five 4th instar cotton aphids (Aphis gossypii) were applied to each plant at 14 days after planting on the 1st true leaf and allowed to reproduce for 7 days. N=18 per endophyte*variety combination. The total number of aphids and the number of winged adult aphids (termed alates) on each plant 7 days after infestation were recorded, exemplary results are shown in FIGS. 81-85.

Results: Total number of aphids on each plant was used as a measure of reproductive success, presumably reflecting the quality of the host to support aphid development and reproduction. The number of winged adults (alates) was also counted. Wing polymorphism is very common in aphids and has been shown to increase in frequency in response to stressful conditions, including changes in host quality. Thus, the number of alates per plant can be interpreted as a potential indicator of the quality of the plant to act as a host to the insect, with a reduction of the host quality of the plants predicted to induce the production of more alates. In the endophyte-aphid experiments, some endophyte treatments clearly reduced total aphid numbers on the plant relative to control, indicating that the endophyte treatment negatively affected aphid reproductive capacity on the plant. Alternatively, some endophyte treatments resulted in an increase in the total number of aphids per plant, suggesting a positive effect of the treatment on the quality of the plant as a host. Some endophyte treatments increased the number of alates produced on the plant, consistent with the prediction of a higher number of alates produced on plants that were less amenable hosts relative to the untreated controls.

Plants treated with the Alternaria eichorniae endophytes TAM00179 (MIC-86713) and TAM00053 (MIC-34397), the Cladosporium cladosporioides endophyte TAM00474 (MIC-34220), Epicoccum nigrum endophyte TAM00089 (MIC-67271), the Chaetomium globosum endophyte TAM00117 (MIC-23475), and Purpureocilhum lavendulum endophyte TAM00424 (MIC-21610) had a greater than 15% reductions in the number of aphids relative to formulation controls indicating negative affects on aphid reproductive capacity and a greater than 12% increase in the number of alates relative to formulation controls indicating reduced attractiveness of the treated plants as aphid hosts. Plants treated with Purpureocilhum lavendulum TAM00424 (MIC-21610) had greater than 70% reductions in the number of aphids relative to formulation controls and greater than 70% increase in the number of alates. Plants treated with Purpureocilhum lavendulum TAM00239 (MIC-86415) also a greater than 30% increase in the number of alates.

TABLE 11
Effect of fungal endophyte treatment on the average number of aphids and alates
on cotton plants as a percent change compared to the formulation control for
that round. The number of alates and aphids were recorded after 7 days of
reproduction on the cotton plants, n = 18 plants per treatment.

| Round | TAMID | TAXID | MICID | Aphid Number | Alates Number |
|---|---|---|---|---|---|
| GH3 | TAM00100 | Epicoccum nigrum | MIC-77047 | 39.73 | −17.65 |
| GH3 | TAM00129 | Alternaria eichorniae | MIC-39830 | 39.21 | −2.48 |
| GH3 | TAM00179 | Alternaria eichorniae | MIC-86713 | 37.64 | 9.38 |
| GH3 | TAM00244 | Cladosporium sp. | MIC-48747 | 18.9 | −27.94 |
| GH3 | TAM00356 | Chaetomium coarctatum | MIC-59232 | 41.66 | −17.65 |
| GH3 | TAM00488 | Gibellulopsis nigrescens | MIC-85153 | 13.1 | −28.32 |
| GH3 | TAM00494 | Cladosporium sp. | MIC-26952 | 34.88 | −38.24 |
| GH3 | TAM00534 | Chaetomium globosum | MIC-33414 | 36.91 | −25.27 |
| GH4 | TAM00089 | Epicoccum nigrum | MIC-67271 | −16.15 | 62.54 |
| GH4 | TAM00129 | Alternaria eichorniae | MIC-39830 | 2.14 | 171.62 |
| GH4 | TAM00317 | Chaetomium sp. | MIC-39772 | −17.62 | −23.8 |
| GH4 | TAM00333 | Chaetomium coarctatum | MIC-39959 | 14.08 | 141.35 |
| GH4 | TAM00340 | Fusarium sp. | MIC-87502 | 22.46 | 139.73 |
| GH4 | TAM00452 | Alternaria eichorniae | MIC-26235 | −56.46 | −33.71 |
| GH4 | TAM00505 | Acremonium alternatum | MIC-96038 | 2.02 | 9.11 |
| GH4 | TAM00514 | Cryptococcus sp. | MIC-39051 | 9.34 | 10.73 |
| GH4 | TAM00560 | Chaetomium globosum | MIC-44512 | −34.2 | −23.21 |
| GH5 | TAM00110 | Chaetomium sp. | MIC-66827 | 8.87 | −18.11 |
| GH5 | TAM00117 | Chaetomium globosum | MIC-23475 | −24.56 | 12.28 |
| GH5 | TAM00137 | Diaporthe sp. | MIC-42067 | 53.98 | 5.77 |
| GH5 | TAM00179 | Alternaria eichorniae | MIC-86713 | −32.68 | 85.5 |
| GH5 | TAM00248 | Cladosporium sp. | MIC-10602 | −32.7 | −32.01 |
| GH5 | TAM00340 | Fusarium sp. | MIC-87502 | 37.43 | −38.73 |
| GH5 | TAM00474 | Cladosporium cladosporioides | MIC-34220 | −15.47 | 47.39 |
| GH5 | TAM00501 | Cladosporium sp. | MIC-31246 | −26.8 | −80.77 |
| GH5 | TAM00565 | Cladosporium herbarum | MIC-20835 | 39.67 | 44.43 |
| GH6 | TAM00033 | Epicoccum nigrum | MIC-16895 | 27.7 | 92.12 |
| GH6 | TAM00053 | Alternaria eichorniae | MIC-34397 | −16.86 | 41.06 |
| GH6 | TAM00072 | Epicoccum nigrum | MIC-55629 | 22.03 | 5.58 |
| GH6 | TAM00087 | Alternaria eichorniae | MIC-78639 | 35 | 51.78 |
| GH6 | TAM00117 | Chaetomium globosum | MIC-23475 | 56.14 | 114.18 |
| GH6 | TAM00125 | Epicoccum nigrum | MIC-51347 | 126.35 | 287.06 |
| GH6 | TAM00128 | Epicoccum nigrum | MIC-65047 | 9.09 | 32.03 |
| GH6 | TAM00131 | Epicoccum nigrum | MIC-85590 | 76.01 | 247.35 |
| GH6 | TAM00239 | Purpureocillium lavendulum | MIC-86415 | 0.23 | 36.54 |
| GH6 | TAM00251 | Chaetomium piluliferum | MIC-51742 | 8.93 | 81.73 |
| GH7 | TAM00169 | Cladosporium herbarum | MIC-91347 | −4.43 | 52.31 |
| GH7 | TAM00190 | Cladosporium herbarum | MIC-42406 | −6.28 | 4.32 |
| GH7 | TAM00193 | Cladosporium cladosporioides | MIC-12927 | −21 | −7.27 |
| GH7 | TAM00413 | Penicillium sp. | MIC-50324 | −71.18 | −57.35 |
| GH7 | TAM00415 | Cladosporium herbarum | MIC-87929 | −19.39 | −15.7 |
| GH7 | TAM00416 | Alternaria eichorniae | MIC-31674 | 11.46 | 67.4 |
| GH7 | TAM00424 | Purpureocillium lavendulum | MIC-21610 | −70.47 | 74.22 |
| GH7 | TAM00508 | | MIC-72092 | −11.96 | −25.18 |
| GH7 | TAM00517 | Cladosporium cladosporioides | MIC-72531 | 9.32 | −4.82 |
| GH8 | TAM00032 | Epicoccum nigrum | MIC-68178 | −34.43 | −21.75 |
| GH8 | TAM00057 | Drechslerella dactyloides | MIC-65885 | −47.62 | −28.01 |
| GH8 | TAM00201 | Cladosporium sp. | MIC-29439 | −44.47 | −32.68 |
| GH8 | TAM00304 | Cladosporium sp. | MIC-17794 | −38.12 | −50.03 |
| GH8 | TAM00489 | | MIC-38632 | −31.33 | −47.54 |
| GH8 | TAM00497 | Epicoccum nigrum | MIC-90504 | −23.58 | 2.06 |
| GH8 | TAM00512 | Crytpococcus | MIC-67609 | −32.31 | −33.94 |
| GH8 | TAM00526 | Phomopsis liquidambari | MIC-95013 | −33.01 | −30.06 |
| GH8 | TAM00529 | Alternaria eichorniae | MIC-61920 | −57.6 | −51.91 |

### Example 11. Southern Green Stink Bug Behavior Assay

Fungal spore suspensions were produced and Phytogen 499 seeds were treated according to the methods of Example 3.

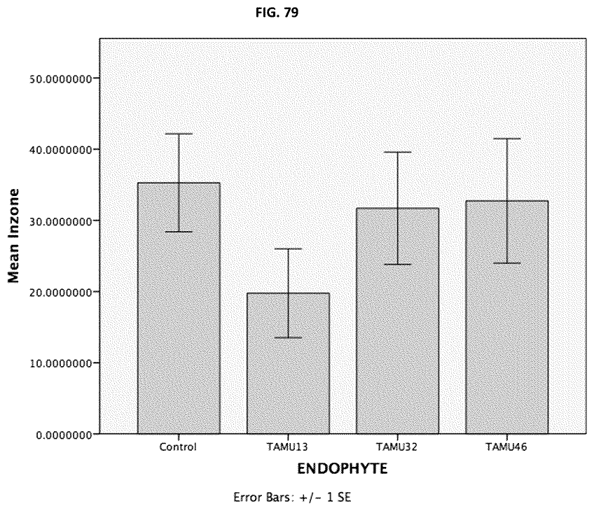

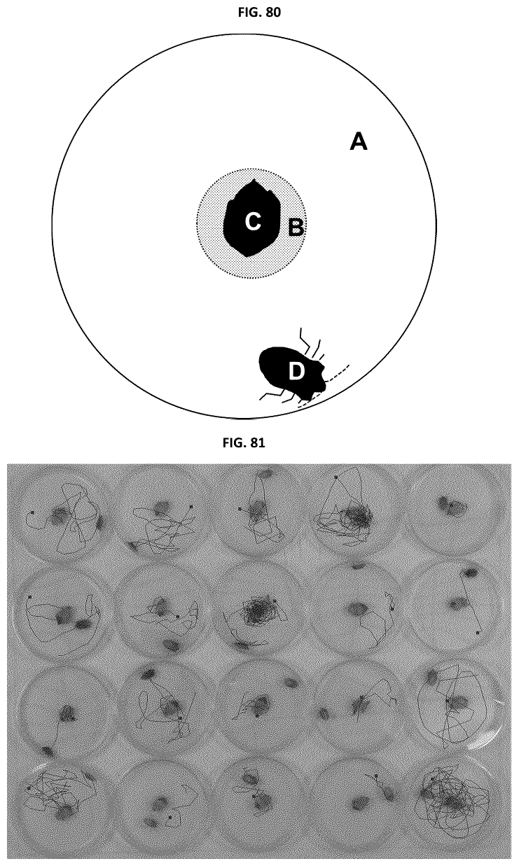

Detached boll assay No-choice behavioral assays were conducted to compare the response of Southern green stink bug (Nezara viridula) individuals to fruits (bolls) from field grown endophyte-treated and untreated cotton plants. The assays were conducted in a temperature controlled observation room at 30° C. in 10 cm diameter Petri dishes with a thin layer of 2% agar on the bottom to provide moisture for the bolls used during the observations. The agar was covered with parafilm to create a dry surface for the insects. For no-choice assays, a single boll was removed from the source plant and pressed into the center of the dish. A single young adult (1-7 d post molt) insect was placed in each dish and covered with the lid. Video tracking software was used to define a “zone” around the boll and tracks insect as it moves in and out of the zone. FIG. 79 shows an exemplary photo of a petri plate “arena” used in this assay and the computer defined zone around each boll. FIG. 80 shows an example of the output of the video tracking software (Ethovision XT version 8.0, Noldus Information Technology, Inc. Leesburg, VA), a visualization of the path over which the insect in that arena has traveled over the observation period. In each trial, 20 insects were observed for each endophyte and control treatment. Petri dish positions were randomized to avoid any positional bias during the observations. The N. viridula no-choice trials were replicated 4 times (total n=20 per treatment) with bolls from field-grown plants. Balanced sex ratios were used in all experiments. No difference between the sexes was observed and data were pooled for final analysis.

Insects in the no-choice assay were observed for 6 hours per trial using video tracking software. For each insect in each trial, the software recorded the insect's movement and the amount of time, if any, spent in the zone surrounding the boll.

Out of 36 fungal endophyte isolates screened in this assay, 10 strains showed greater than 20% reduction in the average amount of time N. viridula spent in contact with bolls compared to bolls collected from formulation treated plants. Two of those strains showed greater than 60% reduction in average boll time compared to formulation controls (Table 12).

Where multiple isolates of the same species were screened, the following patterns were observed. Very few species showed a consistent response across all isolates. For Bipolaris spicifera, 2 isolates showed a decrease in total boll time, while 2 showed an increase in total boll time. For Chaetomium globosum, both isolates showed an increase in total boll time compared to controls. In the case of Cladosporium herbarum, 5 isolates showed a decrease in total boll time while 3 isolates showed an increase in total boll time. Epicoccum nigrum showed 4 isolates that decreased, 2 that had no change and 1 that increased total boll time compared to controls. Gibellulopsis nigrescens showed one isolate decreased and 2 isolates increased boll time. Both Stemphylium herbarum isolates increased total boll time relative to controls. Of the 36 isolates tested, 9 showed a decrease in latency to first contact compared to the formulation controls with 4 of those showing over 100% increase in the amount of time that passed before the insects made first contact with the boll.

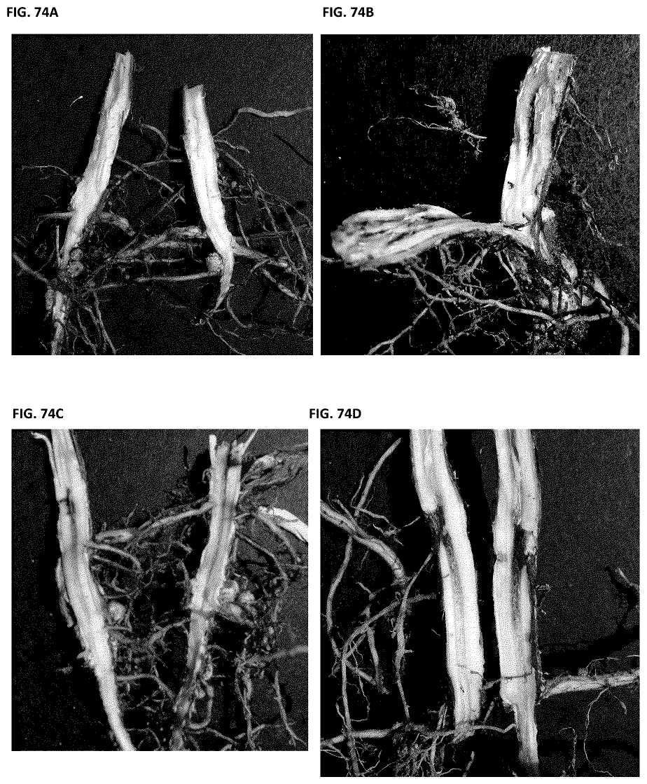

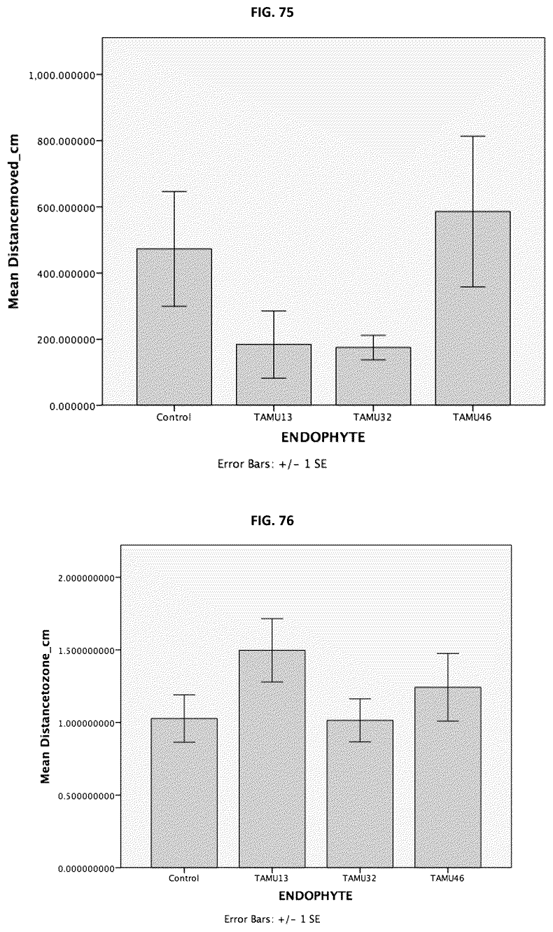

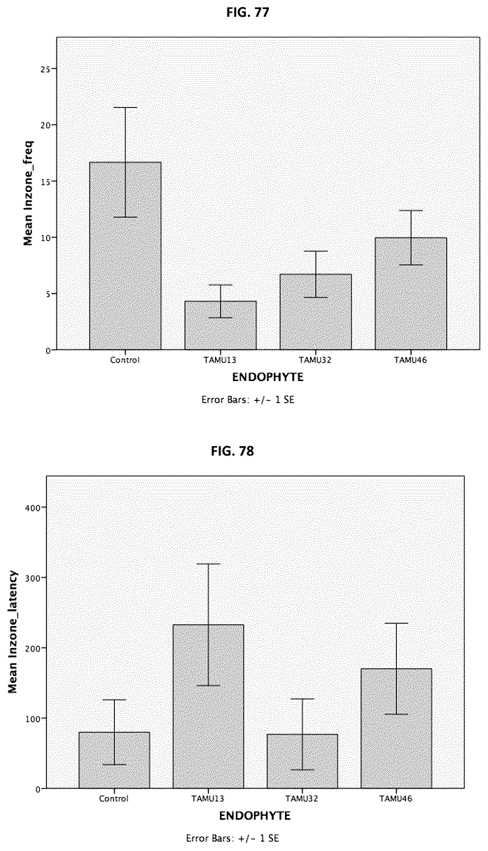

FIG. 76 shows a reduction in the average frequency of visits of N. viridula to bolls from field grown cotton treated with TAM00013 (MIC-92234), TAM00046 (MIC-90694) or TAM00032 (MIC-68178). FIG. 78 shows a reduction in the cumulative time N. viridula spent on bolls from field grown cotton treated with TAM00013 (MIC-92234), TAM00046 (MIC-90694) or TAM00032 (MIC-68178) as a percentage of the total observation period. FIG. 77 shows an increase in the average latency of first contact of N. viridula to bolls from field grown cotton treated with TAM00013 (MIC-92234). FIG. 74 shows a reduction in the mean distance moved by N. viridula in arenas with bolls from field grown cotton treated with TAM00013 (MIC-92234) and TAM00032 (MIC-68178) relative to bolls from untreated reference plants. FIG. 75 shows that on average N. viridula are a greater distance from bolls treated with TAM00013 (MIC-92234) during the observation period relative to bolls from plants not treated with an endophyte.

TABLE 12
Aggregated metrics for a detached cotton boll behavior assay with Nezara viridula expressed for
each fungal endophyte as a percent change compared to the formulation control included in each round.
Video tracking software was used to record time spent on the boll, latency to first contact, and
frequency of boll visits during the 6 hour no choice assay. n = 20 bolls per treatment.

| | | | | | % Change | |
|---|---|---|---|---|---|---|
| | | | | % Change | in Latency | % Change |
| | | | | in Boll | to First | in Boll |
| Round | TAMID | TAXID | MICID | Time | Contact | Frequency |
| 6 | TAM00323 | Alternaria planifunda | MIC-39233 | 2.75 | 92.77 | 1.58 |
| 7 | TAM00439 | Bipolaris spicifera | MIC-77538 | −29.87 | 12.35 | −15.65 |
| 1 | TAM00013 | Bipolaris spicifera | MIC-92234 | −23.96 | −191.48 | −64.95 |
| 4 | TAM00189 | Curvularia spicifera | MIC-31593 | 95.98 | 91.61 | −0.05 |
| 12 | TAM00560 | Chaetomium globosum | MIC-44512 | 52.41 | 19.48 | −12.31 |
| 12 | TAM00554 | Chaetomium globosum | MIC-33414 | 90.61 | 47.46 | −20.47 |
| 6 | TAM00317 | Chaetomium sp. | MIC-39772 | −17.36 | 10.87 | 9.24 |
| 3 | TAM00249 | Cladosporium gossypiicola | MIC-80602 | −68.43 | −45.36 | 35.29 |
| 3 | TAM00248 | Cladosporium sp. | MIC-10602 | −59.64 | 22.88 | −53.27 |
| 11 | TAM00534 | Cladosporium oxysporum | MIC-50414 | −32.02 | 69.25 | 69.67 |
| 5 | TAM00201 | Cladosporium sp. | MIC-29439 | −28.94 | 29.24 | −48.87 |
| 10 | TAM00463 | Cladosporium sp. | MIC-91557 | −23.63 | 17.18 | −49.48 |
| 9 | TAM00501 | Cladosporium sp. | MIC-31246 | 2.37 | 5.87 | −43.57 |
| 8 | TAM00494 | Cladosporium sp. | MIC-26952 | 16.34 | 18.47 | 43.41 |
| 5 | TAM00193 | Cladosporium cladosporioides | MIC-12927 | 30.09 | −44.71 | −50.81 |
| 3 | TAM00105 | Curvularia protuberata | MIC-07010 | −61.37 | −145.98 | −46.06 |
| 2 | TAM00100 | Epicoccum nigrum | MIC-77047 | −30.2 | 72.93 | 69.78 |
| 2 | TAM00103 | Epicoccum nigrum | MIC-62081 | −22.17 | 77.07 | −34.56 |
| 1 | TAM00046 | Didymellaceae | MIC-90694 | −7.19 | −113.06 | −40.24 |
| 2 | TAM00074 | Epicoccum nigrum | MIC-85555 | −0.4 | 33.39 | −30.76 |
| 1 | TAM00032 | Epicoccum nigrum | MIC-68178 | 0.02 | 3.83 | −55.03 |
| 9 | TAM00497 | Epicoccum nigrum | MIC-90504 | −8.09 | −22.54 | −63.08 |
| 12 | TAM00536 | Epicoccum nigrum | MIC-16066 | 30.96 | 57.69 | −13.33 |
| 11 | TAM00531 | Gibellulopsis nigrescens | MIC-16714 | −18.04 | −46.63 | 3.28 |
| 11 | TAM00533 | Gibellulopsis nigrescens | MIC-49739 | 18.27 | 16.95 | 29.6 |
| 10 | TAM00524 | Gibellulopsis nigrescens | MIC-42665 | 44.66 | 40.69 | −44.83 |
| 4 | TAM00251 | Chaetomium piluliferum | MIC-51742 | 28.49 | 24.78 | −26 |
| 8 | TAM00490 | Phialemonium inflatum | MIC-50989 | −10.03 | −105.57 | 52.02 |
| 5 | TAM00194 | Epicoccum nigrum | MIC-76091 | 55.67 | −6.63 | −73.85 |
| 6 | TAM00296 | Pleosporaceae sp. | MIC-12207 | 30.44 | 75.85 | 29.2 |
| 10 | TAM00518 | Preussia africana | MIC-17815 | 43.73 | 38.73 | −15.33 |
| 7 | TAM00402 | Schizothecium inaequale | MIC-39100 | −17.95 | 30.02 | 135.67 |
| 9 | TAM00504 | Sphaerulina pseudovirgaureae | MIC-63740 | −8.43 | 23.89 | −54.79 |
| 4 | TAM00160 | Stagonospora sp. | MIC-20571 | 64.42 | 58.77 | 30.77 |
| 7 | TAM00362 | Stemphylium herbarum | MIC-45943 | −16.91 | 64.14 | 43.63 |
| 8 | TAM00473 | Stemphylium herbarum | MIC-32308 | −11.11 | 60.3 | 22.3 |

### Example 12: Effect of Fungal Endophytes on Hemiptera Insects

Endophyte-treated and control plants are grown from cotton seeds (Gossypium hirsutum) that are inoculated with one or more candidate endophytes (such as Chaetomium globosum e.g., TAM00554 (MIC-33414), Epicoccum nigrum e.g., TAM00194 (MIC-76091), Cladosporium sp. e.g., TAM00463 (MIC-91557)). The plants may be grown under greenhouse and field conditions. Greenhouse plants are first germinated in seedling trays and then transferred to pots. Field grown plants are directly sown in the soil.

Behavioral assays: No-choice and choice behavioral assays are conducted to compare the response of western tarnished plant bugs (Lygus hesperus) and green stink bugs (Nezara viridula) to squares and bolls from endophyte-treated and untreated plants. The assays are conducted at 30° C. in 10 cm diameter petri dishes with a thin layer of 2% agar on the bottom to provide moisture for the squares (L. hesperus assays) and bolls (N. viridula assays) from experimental plants offered to the insects during the observations. For no-choice assays, a single square or boll is inserted by the base into the agar in the center of the dish. A single young adult (1-7 days post molt) insect is placed in each dish and the dish covered with the top. At least 10 insects are observed for each control may be from greenhouse or field grown plants.

Choice assays are conducted in plates as above, but with two equal sized squares (L. hesperus) or bolls (N. viridula) placed 4 ncm apart in the center of the petri dish. One of the two squares or bolls is from an untreated control plant and the other square or boll is from an endophyte treated plant. At least 10 insects are observed for each control and treatment group. served either feeding or resting upon cotton squares (L. hesperus) or bolls (N. viridula) is compared between treatment groups at each observation point across the duration of the assay using the Wilcoxon Signed Ranks Test. To test for variation in responses over time, for each individual the proportion of observations either feeding or upon the plant sample is calculated for early (0-60 min), middle (61-180 min) and late (181-360 min) periods of the assay and compared across treatment groups using a repeated measures analysis of variance (ANOVA) with the endophyte treatment group as the main factor and time as the repeat effect. The observed frequency of individuals failing to make contact with squares or bolls from endophyte-treated plants is compared to the expected frequency of individuals failing to do so based on the control group using a Chi-squared test. Among the insects that did make contact with either a square or boll, the time to first contact (latency) is compared among treatment groups using a one-way ANOVA.

### Example 13. Assessment of Improved Biotic Stress Tolerance of Soy in Field Conditions

Field trials are conducted using chemically treated soy seeds coated with fungal endophytes described herein and formulation control (no endophyte) and untreated controls (no endophyte and no formulation) as described in Example 3. Plots for in-field assessment harbor populations of root knot nematode (Meloidogyne incognita) and Reniform nematode (Rotylenchulus reniformis), respectively, at an approximately 1.0+E04 eggs per gram of fresh root weight. Opportunistically, these plots are infected with natural inoculum of Fusarium virguhforme, the causal agent of Fusarium Sudden Death Syndrome (SDS). Replicate plots, preferably at least 4 replicate plots, are planted per endophyte or control treatment in a randomized complete block design. Each plot consists of a 7.62 m (25 ft.) by 0.76 m (2.5 ft) row. The following early growth metrics are measured: percent emergence at 14 days post planting, standing count at 28 and 45 days post planting, plant vigor at 14, 28, and 45 days post planting, plant height at 45 days post planting, fresh shoot weight, fresh root weight, disease rating at a 0-3 scale (3 denotes strong disease symptoms) using the split-root scoring system at 45 days post planting, nematode count at 45 days post planting, and yield parameters. An exemplary photo of roots receiving scores of 0, 1, 2 and 3 are each shown in FIGS. 85A-D.

At the end of the field trial employing endophyte treatment and control treatment plants, plants (preferably at least 4 plants) are randomly dig out from each row, kept in a plastic bag, and brought back to lab for metric measurements. For each seedling, shoot and root are separated by cutting the seedling 3 cm from the first branch of the root. The heights of the separated shoot of each plant are measured, followed by fresh shoot weight, and fresh root weight. The main root is vertically split into two halves and discoloration of xylem is scored as described above. To extract and count nematode eggs on root, roots are place in a container prefilled with 100 ml 10% sucrose and incubated on a shaker at room temperature overnight. The supernatant is then collected and nematode eggs are counted under a stereomicroscope.

Data are manually curated and entered into ARM database before being analyzed. The percentage of survival plants, fresh root weight, and nematode egg count are plotted as bar graph of mean±95% confidence interval from the mean using the ggplot2 package of R (R Core Team, 2016. R: A language and environment for statistical computing. R Foundation for Statistical Computing, Vienna, Austria. R-project.org/). Plant heights, fresh shoot weight, and disease scores are plotted as jittered dot of mean±nonparametric bootstrap (1000) of 95% confidence interval from the mean using the ggplot2 package of R.

### Example 14. Assessment of Improved Biotic Stress Tolerance of Cotton in Field Conditions

Field trials are conducted using chemically treated cotton seeds coated with fungal endophytes described herein and formulation control (no endophyte) and untreated controls (no endophyte and no formulation) as described in Example 3. Plots for in-field assessment harbor populations of root knot nematode (Meloidogyne incognita) and Reniform nematode (Rotylenchulus reniformis), respectively, at an approximately 1.0+E04 eggs per gram of fresh root weight. Opportunistically, these plots are infected with natural inoculum of Fusarium virguhforme, the causal agent of Fusarium SDS. Replicate plots, preferably at least 4 replicate plots, are planted per endophyte or control treatment in a randomized complete block design. Each plot consists of a 7.62 m (25 ft.) by 0.76 m (2.5 ft) row. The following early growth metrics are measured: percent emergence at 14 days post planting, standing count at 28 and 45 days post planting, plant vigor at 14, 28, and 45 days post planting, plant height at 45 days post planting, fresh shoot weight, fresh root weight, disease rating at a 0-3 scale (3 denotes strong disease symptoms) using the split-root scoring system at 45 days post planting, nematode count at 45 days post planting, and yield parameters.

At the end of the field trial employing endophyte treatment and control treatment plants, plants (preferably at least 4 plants) are randomly dug out from each row, kept in a plastic bag, and brought back to lab for metric measurements. For each seedling, shoot and root are separated by cutting the seedling 3 cm from the first branch of the root. The heights of the separated shoot of each plant are measured, followed by fresh shoot weight, and fresh root weight. The main root is vertically split into two halves and discoloration of xylem are scored as described above. To extract and count nematode eggs on root, roots are placed in a container prefilled with 100 ml 10% sucrose and incubated on a shaker at room temperature overnight. The supernatant is then collected and nematode eggs are counted under a stereomicroscope.

Data are manually curated and entered into ARM database before being analyzed. The percentage of survival plants, fresh root weight, and nematode egg count are plotted as bar graph of mean±95% confidence interval from the mean using the ggplot2 package of R (R Core Team, 2016. R: A language and environment for statistical computing. R Foundation for Statistical Computing, Vienna, Austria. R-project.org/). Plant heights, fresh shoot weight, and disease scores are plotted as jittered dot of mean±nonparametric bootstrap (1000) of 95% confidence interval from the mean using the ggplot2 package of R.

### Example 15: Fungal Endophyte Seed Treatments Alter Traits in Cotton Cultivars in Field Trials

A field trial is conducted using a randomized block design with replicate plots planted with seeds that are inoculated with one or more candidate endophytes (such as Chaetomium globosum e.g., TAM00554 (MIC-33414), Epicoccum nigrum e.g., TAM00194 (MIC-76091), Cladosporium sp. e.g., TAM00463 (MIC-91557)). One or more varieties of cotton seeds may be used to assess variety specific interactions with endophyte treatment and their affect on yield and insect resistance. The plants are grown under standard agricultural practices.

Yield from plots treated with the described microbial compositions is compared relative to the untreated control plots. Thrips damage assessment is scored on a scale, for example a scale from 0-5: 0=no damage, 1=noticeable feeding scars, but no stunting, 2=noticeable feeding and 25% stunting, 3=feeding with blackened leaf terminals and 50% stunting, 4=severe feeding and 75% stunting, and 5=severe feeding and 90% stunting. For fleahoppers, the number of insects per plant is quantified and reported as an average for each plot. Other mid-season plant traits may also be assessed in the field to determine the effect of the described fungal endophyte compositions.

### Example 16: Modulation of Colonization Frequencies of Native Endophytes in Plants Grown from the Fungal Endophyte-Treated Seed

To determine whether endophyte seed treatments could alter the microbiome of the plant grown from the seed, cotton seeds are inoculated with one or more candidate endophytes (such as Chaetomium globosum e.g., MIC-33414, Epicoccum nigrum e.g., MIC-76091, Cladosporium sp. e.g., MIC-91557). The plants may be grown under greenhouse or field conditions under standard agricultural practices. The microbial community of treated and untreated cotton plants may be analyzed by isolating fungi on PDA media from surface-sterilized above-ground stem/leaf tissue and separately from surface sterilized below-ground root tissue. The microbial community of treated and untreated cotton plants may be analyzed by isolating fungal or bacterial DNA from surface-sterilized above-ground stem/leaf tissue and separately from surface sterilized and sequencing the DNA of the community using techniques well known in the art including 16S or ITS community sequencing or metagenomic sequencing.

### Example 17: Modulation of Phytohormone Levels in Plants Grown from the Fungal Endophyte-Treated Seed

To determine whether fungal endophyte seed treatment affects phytohormone levels in plants grown from the seed, tissue is harvested from the root or leaf tissue of cotton plants inoculated with one or more candidate endophytes and untreated controls, under a variety of herbivory treatments. Phytohormone levels for abscisic acid (ABA), tuberonic acid (12-OH-JA, an oxidation product of JA-Ile) (TA), ascorbic acid (AA), 12-Oxophytodienoic acid (a JA precursor) (OPDA), JA isoleucine (JA-Ile), and salicylic acid (SA) are assessed by LC-MS in leaf and root tissues separately. All phytohormone level comparisons are made versus plants in the untreated control group.

1.-44. (canceled)

45. A method for reducing damage due to biotic stress, comprising inoculating plant elements with a formulation comprising a fungal endophyte heterologously disposed to the plant elements, wherein the fungal endophyte comprises a nucleic acid sequence having at least 97% identity to a nucleic acid sequence selected in Table 3, wherein damage due to biotic stress is reduced as compared to plant elements of reference plants not inoculated with the formulation.

46. The method of claim 45, wherein the crop is cotton and the reduction of damage comprises reduced boll damage.

47. The method of claim 46, wherein the reduction of boll damage comprises a decrease in the loss of bolls of about 1%, 2%, 3%, 4%, 5%, 10%, 15%, 20%, 30%, 40%, or 45%.

48. The method of claim 45, wherein the reduction of damage comprises increased leaf area of about 5%, 10%, 15%, 20%, 30%, 40%, or 45%.

49. The method of claim 45, wherein the reduction of damage improves yield as compared to reference plants not inoculated with the formulation.

50.-52. (canceled)

53. The method of claim 45, wherein the fungal endophyte is selected from the group consisting of: Cladosporium, Alternaria, Bipolaris, Chaetomium, Verticillium, Preussia, Pleospora, Epicoccum, or combinations thereof.

54. The method of claim 45, wherein the fungal endophyte comprises a nucleic acid sequence that is at least 97% identical to a nucleic acid sequence selected from the group consisting of SEQ ID NOs: 26-115.

55. The method of claim 45, wherein the formulation comprises at least 1, 2, 3, 4, 5, 6, 7, 8, 9, 10, 11, 12, 13, 14, 15, 16, 17, 18, 19, or 20 endophytes.

56. The method of claim 45, wherein the plant element is a cotton seed or cotton plant.

57. (canceled)

58. The method of claim 45, wherein the plant element is soybean.

59. (canceled)

60. The method of claim 56, further comprising sterilizing the cotton seeds to remove microorganisms prior to combining the seeds with the endophyte composition.

61. The method of claim 45, wherein the biotic stress is caused by a nematode, an aphid, a fleahopper, a Lygus bug, a stink bug, a soy looper, a cabbage looper, a fungus, or combinations thereof.

62. The method of claim 45, wherein the biotic stress is caused by root knot nematode or reniform nematode.

63. (canceled)

64. The method of claim 45, wherein the biotic stress is caused by a Lepidoptera larvae.

65. The method of claim 64, wherein the biotic stress is caused by a Lepidoptera larvae of the family Noctuidae.

66. The method of claim 65, wherein the biotic stress is caused by Chrysodeixis includens or Trichoplusia ni.

67. (canceled)

68. The method of claim 45, wherein the biotic stress is caused by a Hemiptera insect.

69. The method of claim 68, wherein the biotic stress is caused by Nezara viridula, Lygus Hesperus, or Aphis gossypii.

70.-71. (canceled)

72. The method of claim 45, wherein the biotic stress is caused by a fungi of the genus Rhizoctonia.

73. The method of claim 72, wherein the biotic stress is caused by Rhizoctonia solani.

74. The method of claim 45, wherein the biotic stress is caused by a fungi of the genus Fusarium.

75. The method of claim 74, wherein the biotic stress is caused by Fusarium virguhforme or Fusarium oxysporum.

76.-87. (canceled)


SEQUENCE LISTING (115 sequences)

SEQ ID NO: 1 - DNA, 22 bp, synthetic construct
  misc_feature 1..22: note = primer - ITS_1
  source 1..22: mol_type = other DNA
cttggtcatttagaggaagtaa

SEQ ID NO: 2 - DNA, 20 bp, synthetic construct
  misc_feature 1..20: note = primer - ITS_2
  source 1..20: mol_type = other DNA
gctgcgttcttcatcgatgc

SEQ ID NO: 3 - DNA, 20 bp, synthetic construct
  misc_feature 1..20: note = primer - ITS_3
  source 1..20: mol_type = other DNA
gcatcgatgaagaacgcagc

SEQ ID NO: 4 - DNA, 17 bp, synthetic construct
  misc_feature 1..17: note = primer - LR5
  source 1..17: mol_type = other DNA
tcctgagggaaacttcg

SEQ ID NO: 5 - DNA, 20 bp, synthetic construct
  misc_feature 1..20: note = primer - amplicon F
  source 1..20: mol_type = other DNA
ggtgaatcgcacatgctaga

SEQ ID NO: 6 - DNA, 20 bp, synthetic construct
  misc_feature 1..20: note = primer - amplicon R
  source 1..20: mol_type = other DNA
cgaccagacagagcgtatga

SEQ ID NO: 7 - DNA, 23 bp, synthetic construct
  misc_feature 1..23: note = primer - bRPB2-7.1R
  source 1..23: mol_type = other DNA
cccatrgcytgyttmcccatdgc

SEQ ID NO: 8 - DNA, 20 bp, synthetic construct
  misc_feature 1..20: note = primer - fRPB2-7R
  source 1..20: mol_type = other DNA
cccatwgcytgcttmcccat

SEQ ID NO: 9 - DNA, 20 bp, synthetic construct
  misc_feature 1..20: note = primer - fRPB2-5F
  source 1..20: mol_type = other DNA
gaygaymgwgatcayttygg

SEQ ID NO: 10 - DNA, 17 bp, synthetic construct
  misc_feature 1..17: note = primer - PGK_533-F
  source 1..17: mol_type = other DNA
gtygayttcaaygtycc

SEQ ID NO: 11 - DNA, 20 bp, synthetic construct
  misc_feature 1..20: note = primer - PGK_533-R
  source 1..20: mol_type = other DNA
acaccdggdggrccgttcca

SEQ ID NO: 12 - DNA, 21 bp, synthetic construct
  misc_feature 1..21: note = primer - 60S-506F
  source 1..21: mol_type = other DNA
cttvavytggaacttgatggt

SEQ ID NO: 13 - DNA, 19 bp, synthetic construct
  misc_feature 1..19: note = primer - 60S-908R
  source 1..19: mol_type = other DNA
ghgacaagcgtttctcngg

SEQ ID NO: 14 - DNA, 23 bp, synthetic construct
  misc_feature 1..23: note = primer - Btub2Fd
  source 1..23: mol_type = other DNA
gtbcacctycaraccggycartg

SEQ ID NO: 15 - DNA, 26 bp, synthetic construct
  misc_feature 1..26: note = primer - Btub4Rd
  source 1..26: mol_type = other DNA
ccrgaytgrccraaracraagttgtc

SEQ ID NO: 16 - DNA, 19 bp, synthetic construct
  misc_feature 1..19: note = primer - ACT512f
  source 1..19: mol_type = other DNA
atgtgcaaggccggtttcg

SEQ ID NO: 17 - DNA, 20 bp, unidentified
  misc_feature 1..20: note = primer - ACT783r
  source 1..20: mol_type = other DNA
tacgagtccttctggcccat

SEQ ID NO: 18 - DNA, 17 bp, synthetic construct
  misc_feature 1..17: note = primer - LR0R
  source 1..17: mol_type = other DNA
acccgctgaacttaagc

SEQ ID NO: 19 - DNA, 20 bp, synthetic construct
  misc_feature 1..20: note = primer - SSU_NS4
  source 1..20: mol_type = other DNA
cttccgtcaattcctttaag

SEQ ID NO: 20 - DNA, 19 bp, synthetic construct
  misc_feature 1..19: note = primer - SSU_NS1
  source 1..19: mol_type = other DNA
gtagtcatatgcttgtctc

SEQ ID NO: 21 - DNA, 21 bp, synthetic construct
  misc_feature 1..21: note = primer - SSU_SR1R
  source 1..21: mol_type = other DNA
tacctggttgatcctgccagt

SEQ ID NO: 22 - DNA, 20 bp, synthetic construct
  misc_feature 1..20: note = primer - amplicon F
  source 1..20: mol_type = other DNA
ctcctcctcctcctcctgat

SEQ ID NO: 23 - DNA, 20 bp, synthetic construct
  misc_feature 1..20: note = primer - amplicon R
  source 1..20: mol_type = other DNA
tcacagagctacgcgacttg

SEQ ID NO: 24 - DNA, 20 bp, synthetic construct
  misc_feature 1..20: note = primer - RPB1-Af
  source 1..20: mol_type = other DNA
gartgyccdggdcayttygg

SEQ ID NO: 25 - DNA, 23 bp, synthetic construct
  misc_feature 1..23: note = primer - RPB1-Cr
  source 1..23: mol_type = other DNA
ccngcdatntcrttrtccatrta

SEQ ID NO: 26 - DNA, 1415 bp, unidentified
  source 1..1415: mol_type = other DNA; note = Cladosporium cladosporioides
tcttggtcatttagaggaagtaaaagtcgtaacaaggtctccgtaggtgaacctgcggagggatcattacaagtgaccccggtctaaccaccgggatgttcataaccctttgttgtccgactctgttgcctccggggcgaccctgccttcgggcgggggctccgggtggacacttcaaactcttgcgtaactttgcagtctgagtaaacttaattaataaattaaaacttttaacaacggatctcttggttctggcatcgatgaagaacgcagcgaaatgcgataagtaatgtgaattgcagaattcagtgaatcatcgaatctttgaacgcacattgcgccccctggtattccggggggcatgcctgttcgagcgtcatttcaccactcaagcctcgcttggtattgggcatcgcggtccgccgcgtgcctcaaatcgaccggctgggtcttctgtcccctaagcgttgtggaaactattcgctaaagggtgttcgggaggctacgccgtaaaacaaccccatttctaaggttgacctcggatcaggtagggatacccgctgaacttaagcatatcaataagcggaggaaaagaaaccaacagggattgctctagtaacggcgagtgaagcagcaatagctcaaatttgaaatctggcgtcttcgacgtccgagttgtaatttgtagaggatgcttctgagtaaccaccgacctaagttccttggaacaggacgtcatagagggtgagaatcccgtatgcggtcggaaaggtgctctatacgtagctccttcgacgagtcgagttgtttgggaatgcagctctaaatgggaggtaaatttcttctaaagctaaatattggccagagaccgatagcgcacaagtagagtgatcgaaagatgaaaagcactttggaaagagagttaaaaagcacgtgaaattgttaaaagggaagggattgcaaccagacttgctcgcggtgttccgccggtcttctgaccggtctactcgccgcgttgcaggccagcatcgtctggtgccgctggataagacttgaggaatgtagctccttcgggagtgttatagcctcttgtgatgcagcgagcgccgggcgaggtccgcgcttcggctaggatgctggcgtaatggtcgtaatccgcccgtcttgaaacacggaccaaggagtctaacatctatgcgagtgttcgggtgtcaaacccctacgcgtaatgaaagtgaacggaggtgagaaccgcaaggtgcatcatcgaccgatcctgatgtcttcggatggatttgagtaagagcatagctgttgggacccgaaagatggtgaactatgcctgaatagggtgaagccagaggaaactctggcggaggctcgcagcggttccgacgtgcaaatcgatcgtcaaa

SEQ ID NO: 27 - DNA, 493 bp, unidentified
  source 1..493: mol_type = other DNA; note = Cladosporium cladosporioides
gtctacccccgggatgttcataaccctttgttgtccgactctgttgcctccggggcgaccctgccttcgggcgggggctccgggtggacacttcaaactcttgcgtaactttgcagtctgagtaaacttgattaataaattaaaacttttaacaacggatctcttggttctggcatcgatgaagaacgcagcgaaatgcgataagtaatgtgaattgcagaattcagtgaatcatcgaatctttgaacgcacattgcgccccctggtattccggggggcatgcctgttcgagcgtcatttcaccactcaagcctcgcttggtattgggcaacgcggtccgccgcgtgcctcaaatcgaccggctgggtcttctgtcccctaagcgttgtggaaactattcgctaaagggtgctcgggaggctacgccgtaaaacaaacccatttctaaggttgacctcggatcaggtagggatacccgctgaacttaagcata

SEQ ID NO: 28 - DNA, 484 bp, unidentified
  source 1..484: mol_type = other DNA; note = Cladosporium cladosporioides
ccgggatgttcataaccctttgttgtccgactctgttgcctccggggcgaccctgccttcgggcgggggctccgggtggacacttcaaactcttgcgtaactttgcagtctgagtaaacttaattaataaattaaaacttttaacaacggatctcttggttctggcatcgatgaagaacgcagcgaaatgcgataagtaatgtgaattgcagaattcagtgaatcatcgaatctttgaacgcacattgcgccccctggtattccggggggcatgcctgttcgagcgtcatttcaccactcaagcctcgcttggtattgggcaacgcggtccgccgcgtgcctcaaatcgaccggctgggtcttctgtcccctaagcgttgtggaaactattcgctaaagggtgttcgggaggctacgccgtaaaacaaccccatttctaaggttgacctcggatcaggtagggatacccgctgaacttaagcat

SEQ ID NO: 29 - DNA, 494 bp, unidentified
  misc_feature 1..494: note = Kingdom: Fungi, Phylum: Ascomycota, Class: Dothideomycetes, Order: Capnodiales, Family: Cladosporiaceae, Genus: Cladosporium
  source 1..494: mol_type = other DNA
cggtctaccaccgggatgttcataaccctttgttgtccgactctgttgcctccggggcgaccctgccttcgggcgggggctccgggtggacacttcaaactcttgcgtaactttgcagtctgagtaaacttaattaataaattaaaacttttaacaacggatctcttggttctggcatcgatgaagaacgcagcgaaatgcgataagtaatgtgaattgcagaattcagtgaatcatcgaatctttgaacgcacattgcgccccctggtattccggggggcatgcctgttcgagcgtcatttcaccactcaagcctcgcttggtattgggcaacgcggtccgccgcgtgcctcaaatcgaccggctgggtcttctgtcccctaagcgttgtggaaactattcgctaaagggtgttcgggaggctacgccgtaaaacaaccccatttctaaggttgacctcggatcaggtagggatacccgctgaacttaagcat

SEQ ID NO: 30 - DNA, 163 bp, unidentified
  source 1..163: mol_type = other DNA; note = Cladosporium cladosporioides
taaccctttgttgtccgactctgttgcctccggggcgaccctgccttcgggcgggggctccgggtggacacttcaaactcttgcgtaactttgcagtctgagtaaatttaattaataaattaaaacttttaacaacggatctcttggttctggcatcgatgaa

SEQ ID NO: 31 - DNA, 484 bp, unidentified
  source 1..484: mol_type = other DNA; note = Cladosporium gossypiicola
ccgggatgttcataaccctttgttgtccgactctgttgcctccggggcgaccctgccttcgggcgggggctccgggtggacacttcaaactcttgcgtaactttgcagtctgagtaaacttaattaataaattaaaacttttaacaacggatctcttggttctggcatcgatgaagaacgcagcgaaatgcgataagtaatgtgaattgcagaattcagtgaatcatcgaatctttgaacgcacattgcgccccctggtattccggggggcatgcctgttcgagcgtcatttcaccactcaagcctcgcttggtattgggcatcgcggtccgccgcgtgcctcaaatcgaccggctgggtcttctgtcccctaagcgttgtggaaactattcgctaaagggtgttcgggaggctacgccgtaaaacaaccccatttctaaggttgacctcggatcaggtagggatacccgctgaacttaagcat

SEQ ID NO: 32 - DNA, 486 bp, Cladosporium herbarum
  source 1..486: mol_type = other DNA
ccgggatgttcataaccctttgttgtccgactctgttgcctccggggcgaccctgccttcgggcgggggctccgggtggacacttcaaactcttgcgtaactttgcagtctgagtaaacttaattaataaattaaaacttttaacaacggatctcttggttctggcatcgatgaagaacgcagcgaaatgcgataagtaatgtgaattgcagaattcagtgaatcatcgaatctttgaacgcacattgcgccccctggtattccggggggcatgcctgttcgagcgtcatttcaccactcaagcctcgcttggtattgggcaacgcggtccgccgcgtgcctcaaatcgaccggctgggtcttctgtcccctaagcgttgtggaaactattcgctaaagggtgctcgggaggctacgccgtaaaacaaacccatttctaaggttgacctcggatcaggtagggatacccgctgaacttaagcatat

SEQ ID NO: 33 - DNA, 485 bp, Cladosporium herbarum
  source 1..485: mol_type = other DNA
ccgggatgttcataaccctttgttgtccgactctgttgcctccggggcgaccctgccttcgggcgggggctccgggtggacacttcaaactcttgcgtaactttgcagtctgagtaaatttaattaataaattaaaacttttaacaacggatctcttggttctggcatcgatgaagaacgcagcgaaatgcgataagtaatgtgaattgcagaattcagtgaatcatcgaatctttgaacgcacattgcgccccctggtattccggggggcatgcctgttcgagcgtcatttcaccactcaagcctcgcttggtattgggcaacgcggtccgccgcgtgcctcaaatcgaccggctgggtcttctgtcccctaagcgttgtggaaactattcgctaaagggtgctcgggaggctacgccgtaaaacaaacccatttctaaggttgacctcggatcaggtagggatacccgctgaacttaagcata

SEQ ID NO: 34 - DNA, 1445 bp, unidentified
  source 1..1445: mol_type = other DNA; note = Cladosporium oxysporum
tccgtaggtgaacctgcggagggatcattacaagtgaccccggtctaaccaccgggatgttcataaccctttgttgtccgactctgttgcctccggggcgaccctgccttcgggcgggggctccgggtggacacttcaaactcttgcgtaactttgcagtctgagtaaacttaattaataaattaaaacttttaacaacggatctcttggttctggcatcgatgaagaacgcagcgaaatgcgataagtaatgtgaattgcagaattcagtgaatcatcgaatctttgaacgcacattgcgccccctggtattccggggggcatgcctgttcgagcgtcatttcaccactcaagcctcgcttggtattgggcaacgcggtccgccgcgtgcctcaaatcgaccggctgggtcttctgtcccctaagcgttgtggaaactattcgctaaagggtgctcgggaggctacgccgtaaaacaaacccatttctaaggttgacctcggatcaggtagggatacccgctgaacttaagcatatcaataagcggaggaaaagaaaccaacagggattgctctagtaacggcgagtgaagcagcaatagctcaaatttgaaatctggcgtcttcgacgtccgagttgtaatttgtagaggatgcttctgagtaaccaccgacctaagttccttggaacaggacgtcatagagggtgagaatcccgtatgcggtcggaaaggtgctctatacgtagctccttcgacgagtcgagttgtttgggaatgcagctctaaatgggaggtaaatttcttctaaagctaaatattggccagagaccgatagcgcacaagtagagtgatcgaaagatgaaaagcactttggaaagagagttaaaaagcacgtgaaattgttaaaagggaagggattgcaaccagacttgctcgcggtgttccgccggtcttctgaccggtctactcgccgcgttgcaggccagcatcgtctggtgccgctggataagacttgaggaatgtagctccctcgggagtgttatagcctcttgtgatgcagcgagcgccgggcgaggtccgcgcttcggctaggatgctggcgtaatggtcgtaatccgcccgtcttgaaacacggaccaaggagtctaacatctatgcgagtgttcgggtgtcaaacccctacgcgtaatgaaagtgaacggaggtgagaaccgcaaggtgcatcatcgaccgatcctgatgtcttcggatggatttgagtaagagcatagctgttgggacccgaaagatggtgaactatgcctgaatagggtgaagccagaggaaactctggtggaggctcgcagcggttctgacgtgcaaatcgatcgtcaaatttgggtataggggcgaaagactaatcgaaccatctagtagctggttcctgccgaagtttccctcagga

SEQ ID NO: 35 - DNA, 496 bp, unidentified
  misc_feature 1..496: note = Kingdom: Fungi, Phylum: Ascomycota, Class: Dothideomycetes, Order: Capnodiales, Family: Cladosporiaceae, Genus: Cladosporium
  source 1..496: mol_type = other DNA
gtctaccaccgggatgttcataaccctttgttgtccgactctgttgcctccggggcgaccctgccttcgggcgggggctccgggtggacacttcaaactcttgcgtaactttgcagtctgagtaaacttaattaataaattaaaacttttaacaacggatctcttggttctggcatcgatgaagaacgcagcgaaatgcgataagtaatgtgaattgcagaattcagtgaatcatcgaatctttgaacgcacattgcgccccctggtattccggggggcatgcctgttcgagcgtcatttcaccactcaagcctcgcttggtattgggcatcgcggtccgccgcgtgcctcaaatcgaccggctgggtcttctgtcccctaagcgttgtggaaactattcgctaaagggtgttcgggaggctacgccgtaaaacaaccccatttctaaggttgacctcggatcaggtagggatacccgctgaacttaagcatatca

SEQ ID NO: 36 - DNA, 492 bp, unidentified
  misc_feature 1..492: note = Kingdom: Fungi, Phylum: Ascomycota, Class: Dothideomycetes, Order: Capnodiales, Family: Cladosporiaceae, Genus: Cladosporium
  source 1..492: mol_type = other DNA
gtctaccaccgggatgttcataaccctttgttgtccgactctgttgcctccggggcgaccctgccttcgggcgggggctccgggtggacacttcaaactcttgcgtaactttgcagtctgagtaaacttaattaataaattaaaacttttaacaacggatctcttggttctggcatcgatgaagaacgcagcgaaatgcgataagtaatgtgaattgcagaattcagtgaatcatcgaatctttgaacgcacattgcgccccctggtattccggggggcatgcctgttcgagcgtcatttcaccactcaagcctcgcttggtattgggcatcgcggtccgccgcgtgcctcaaatcgaccggctgggtcttctgtcccctaagcgttgtggaaactattcgctaaagggtgttcgggaggctacgccgtaaaacaaccccatttctaaggttgacctcggatcaggtagggatacccgctgaacttaagcat

SEQ ID NO: 37 - DNA, 163 bp, unidentified
  misc_feature 1..163: note = Kingdom: Fungi, Phylum: Ascomycota, Class: Dothideomycetes, Order: Capnodiales, Family: Cladosporiaceae, Genus: Cladosporium
  source 1..163: mol_type = other DNA
gccttgctgaattattcacccttgtcttttgcgtacttcttgtttccttggtgggttcgcccaccactaggacaaacataaaccttttgtaattgcaatcagcgtcagtaacaaattaataattacaactttcaacaacggatctcttggttctggcatcgat

SEQ ID NO: 38 - DNA, 496 bp, unidentified
  misc_feature 1..496: note = Kingdom: Fungi, Phylum: Ascomycota, Class: Dothideomycetes, Order: Capnodiales, Family: Cladosporiaceae, Genus: Cladosporium
  source 1..496: mol_type = other DNA
ggtctaccaccgggatgttcataaccctttgttgtccgactctgttgcctccggggcgaccctgccttcgggcgggggctccgggtggacacttcaaactcttgcgtaactttgcagtctgagtaaacttaattaataaattaaaacttttaacaacggatctcttggttctggcatcgatgaagaacgcagcgaaatgcgataagtaatgtgaattgcagaattcagtgaatcatcgaatctttgaacgcacattgcgccccctggtattccggggggcatgcctgttcgagcgtcatttcaccactcaagcctcgcttggtattgggcaacgcggtccgccgcgtgcctcaaatcgaccggctgggtcttctgtcccctaagcgttgtggaaactattcgctaaagggtgctcgggaggctacgccgtaaaacaaacccatttctaaggttgacctcggatcaggtagggatacccgctgaacttaagcatatc

SEQ ID NO: 39 - DNA, 490 bp, unidentified
  misc_feature 1..490: note = Kingdom: Fungi, Phylum: Ascomycota, Class: Dothideomycetes, Order: Capnodiales, Family: Cladosporiaceae, Genus: Cladosporium
  source 1..490: mol_type = other DNA
gtctaccaccgggatgttcataaccctttgttgtccgactctgttgcctccggggcgaccctgccttcgggcgggggctccgggtggacacttcaaactcttgcgtaactttgcagtctgagtaaacttgattaataaattaaaacttttaacaacggatctcttggttctggcatcgatgaagaacgcagcgaaatgcgataagtaatgtgaattgcagaattcagtgaatcatcgaatctttgaacgcacattgcgccccctggtattccggggggcatgcctgttcgagcgtcatttcaccactcaagcctcgcttggtattgggcaacgcggtccgccgcgtgcctcaaatcgaccggctgggtcttctgtcccctaagcgttgtggaaactattcgctaaagggtgctcgggaggctacgccgtaaaacaaacccatttctaaggttgacctcggatcaggtagggatacccgctgaacttaagc

SEQ ID NO: 40 - DNA, 476 bp, unidentified
  source 1..476: mol_type = other DNA; note = Sphaerulina pseudovirgaureae
ggctcgacctccaaccctttgtgaacacaacttgttgcttcgggggcgaccctgccgtttcgacggcgagcgcccccggaggccttcaaacactgcatctttgcgtcggagtttaagtaaattaaacaaaactttcaacaacggatctcttggttctggcatcgatgaagaacgcagcgaaatgcgataagtaatgtgaattgcagaattcagtgaatcatcgaatctttgaacgcacattgcgccccttggtattccgaggggcatgcctgttcgagcgtcatttcaccactcaagcctcgcttggtattgggcgccgcggtgttccgcgcgcctcaaagtctccggctgagctgtccgtctctaagcgttgtgatttcattaatcgcttcggagcgcgggcggtcgcggccgttaaatctttcacaaggttgacctcggatcaggtagggatacccgctgaacttaagcatatc

SEQ ID NO: 41 - DNA, 1446 bp, unidentified
  source 1..1446: mol_type = other DNA; note = Epicoccum nigrum
tcttggtcatttagaggaagtaaaagtcgtaacaaggtttccgtaggtgaacctgcggaaggatcattacctagagtttgtggacttcggtctgctacctcttacccatgtcttttgagtaccttcgtttcctcggcgggtccgcccgccggttggacaacattcaaaccctttgcagttgcaatcagcgtctgaaaaaacttaatagttacaactttcaacaacggatctcttggttctggcatcgatgaagaacgcagcgaaatgcgataagtagtgtgaattgcagaattcagtgaatcatcgaatctttgaacgcacattgcgccccttggtattccatggggcatgcctgttcgagcgtcatttgtaccttcaagctctgcttggtgttgggtgttttgtctcgcctccgcgcgcagactcgccttaaaacaattggcagccggcgtattgatttcggagcgcagtacatctcgcgctttgcactcataacgacgacgtccaaaagtacatttttacactcttgacctcggatcaggtagggatacccgctgaacttaagcatatcaataagcggaggaaaagaaaccaacagggattgccctagtaacggcgagtgaagcggcaacagctcaaatttgaaatctggcgtctttggcgtccgagttgtaatttgcagagggcgctttggcattggcagcggtccaagttccttggaacaggacgtcacagagggtgagaatcccgtacgtggtcgctagcctttaccgtgtaaagccccttcgacgagtcgagttgtttgggaatgcagctctaaatgggaggtaaatttcttctaaagctaaatactggccagagaccgatagcgcacaagtagagtgatcgaaagatgaaaagcactttggaaagagagttaaaaagcacgtgaaattgttgaaagggaagcgcttgcagccagacttgcctgtagttgctcatccgggtttctacccggtgcactcttctacgggcaggccagcatcagtttgggcggttggataaaggtctctgtcatgtacctcccttcggggagatcttataggggagacgacatgcaaccagcctggactgaggtccgcgcatctgctaggatgctggcgtaatggctgtaagcggcccgtcttgaaacacggaccaaggagtctaacatctatgcgagtgtttgggtgtcaagcccgagcgcgtaatgaaagtgaacggaggtgggaacctttcggggtgcaccatcgaccgatcctgatgtcttcggatggatttgagtaagagcatagctgttgggacccgaaagatggtgaactatgcttgaatagggtgaagccagaggaaactctggtggaggctcgcagcggttctgacgtgcaaatcgatcgtcaaatttgggcataggggcgaaagactaatcga

SEQ ID NO: 42 - DNA, 1188 bp, unidentified
  source 1..1188: mol_type = other DNA; note = Epicoccum nigrum
gacgatcgtgaccacttcggtaagaagcgtctcgatcttgctgggccgctgatcgcgaacttattccgcatcctctttttgaagttgaccaaggacgtttacaagtacctccagaagtgcgtcgagaccaattccgacttcaacgtccagatggctgttagagctagtgtcattactaatggtctgaaatattcgctcgccacgggaaactggggtgatcagaagaaggctgcctcagcgaaagctggcgtctcccaggtcttgaaccgttatacgtacgcatctacgttgtcgcatttgcgacgaacaaacactccggtcggccgtgacggcaagcttgcaaagccccgtcagttgcacaacagtcactggggtcttgtatgccccgccgagacacctgaaggacaggcttgtggtcttgtcaagaacttgtcgctgatgtgctacgtcagtgttggtagcgatgccgggcccatatccgagttcatgaagcagcgaaacatgcaggtccttgaagaatatgatcaagcccaaaatcccgatgccaccaaggtcttcgtcaacggtgtatgggtcggtgttcactccaacgcgcagcagctcgtctccacagtacaggagcttcgccgcaatggaaccctatcctacgagatgagtctgattcgagacatccgtgaccgagagttcaaaatctttacagatgctggacgtgttatgaggcctcttttcgtagtggagaacgacgtgcgcaagccgaataggaatcacctcgtctacaaccaagaacactacaacaaactagcccaagagcaaatggcaatgactcaggcaggcgtcggcgaggaagagaagcagccttatggttggaagggcctgatccaagatggtatcatcgaataccttgacgctgaagaggaggagacggccatgattgtcatgtcgcccgaagatcttggcgagtggcgtgatatgaaaatgggcatcccacaagatgctcgcaacccacaaggcaaagaccgtcttgcacgtatcaagcctaagccagatcctcgcatttatgcctacacgcattgcgagatccaccctgctatgattctcggtatctgtgccagtatcattccttttccggaccacaaccagtcgcctcgtaacacataccagtctgctatgggtaagcaagccatgggt

SEQ ID NO: 43 - DNA, 383 bp, unidentified
  source 1..383: mol_type = other DNA; note = Epicoccum nigrum
gttcacctccagaccggtcagtgcgtaagtacaccacttgcatgttctctcatggacggcagctcacaagggacagggtaaccaaatcggtgctgccttctggcagaccatctccggcgagcatggcctcgacggctccggtgtctacaacggcacctcggacctccagctcgagcgcatgaacgtctacttcaacgaggtactagaaacgacacgctttcctgagacggactgcgagtgctgacctcccgcaggcctctggcaacaagttcgttccccgtgccgtcctcgtcgacttggagcccggtacaatggacgctgtccgcgctggccccttcggccagctcttccgtcccgataacttcgtcttcggccagtctggt

SEQ ID NO: 44 - DNA, 400 bp, unidentified
  source 1..400: mol_type = other DNA; note = Epicoccum nigrum
gtgacaagcgtttctctggaactgtcaagctccccaccgttccccgtccccgtaagttgcacgcgcccaaggattgcagtggcaagagctgacagttttgcaggcatggccatctgcgttctcggtgaccagcacgatatcgatcgtgccaagcaccacggtgttgatgccatgtccactgaggacttgaagaagctcaacaagaacaagaagctcatcaagaagctcgcacgcaagtacgatgctttcatggcttccgacgccctgatcaagcagatcccccgtctcttgggtcccggtctgtccaaggctggaaagttccccactcccgtctctcacgctgaggacctcgccaacaagatgaccgatgtcaagtccaccatcaagttccagctgaaga

SEQ ID NO: 45 - DNA, 272 bp, unidentified
  source 1..272: mol_type = other DNA; note = Epicoccum nigrum
tacgagtccttctgtcccataccgatcatgatactgcgtcacggttagctcggggggcaggagctgccaaggagagcgtcatacccatggtgacggggacgaccgacaatggaggctgcaggctgttaggacatgctcgcgaaggtggcctggcgacagggcgggcgcaatgcatggctgcttatgctcccgctgctgccaggtggcgggttggggacttacggaagactgctcggggcgcatcatcaccggcgaaaccggccttgcacata

SEQ ID NO: 46 - DNA, 1447 bp, unidentified
  source 1..1447: mol_type = other DNA; note = Epicoccum nigrum
tccgtaggtgaacctgcggaaggatcattacctagagtttgtggacttcggtctgctacctcttacccatgtcttttgagtaccttcgtttcctcggcgggtccgcccgccggttggacaacattcaaaccctttgcagttgcaatcagcgtctgaaaaaacttaatagttacaactttcaacaacggatctcttggttctggcatcgatgaagaacgcagcgaaatgcgataagtagtgtgaattgcagaattcagtgaatcatcgaatctttgaacgcacattgcgccccttggtattccatggggcatgcctgttcgagcgtcatttgtaccttcaagctctgcttggtgttgggtgttttgtctcgcctccgcgcgcagactcgccttaaaacaattggcagccggcgtattgatttcggagcgcagtacatctcgcgctttgcactcataacgacgacgtccaaaagtacatttttacactcttgacctcggatcaggtagggatacccgctgaacttaagcatatcaataagcggaggaaaagaaaccaacagggattgccctagtaacggcgagtgaagcggcaacagctcaaatttgaaatctggcgtctttggcgtccgagttgtaatttgcagagggcgctttggcattggcagcggtccaagttccttggaacaggacgtcacagagggtgagaatcccgtacgtggtcgctagcctttaccgtgtaaagccccttcgacgagtcgagttgtttgggaatgcagctctaaatgggaggtaaatttcttctaaagctaaatactggccagagaccgatagcgcacaagtagagtgatcgaaagatgaaaagcactttggaaagagagttaaaaagcacgtgaaattgttgaaagggaagcgcttgcagccagacttgcctgtagttgctcatccgggtttctacccggtgcactcttctacgggcaggccagcatcagtttgggcggttggataaaggtctctgtcatgtacctcccttcggggagatcttataggggagacgacatgcaaccagcctggactgaggtccgcgcatctgctaggatgctggcgtaatggctgtaagcggcccgtcttgaaacacggaccaaggagtctaacatctatgcgagtgtttgggtgtcaagcccgagcgcgtaatgaaagtgaacggaggtgggaacctttcggggtgcaccatcgaccgatcctgatgtcttcggatggatttgagtaagagcatagctgttgggacccgaaagatggtgaactatgcttgaatagggtgaagccagaggaaactctggtggaggctcgcagcggttctgacgtgcaaatcgatcgtcaaatttgggcataggggcgaaagactaatcgaactatctagtagctggttcctgccgaagtttccctcagga

SEQ ID NO: 47 - DNA, 1439 bp, unidentified
  source 1..1439: mol_type = other DNA; note = Epicoccum nigrum
tcttggtcatttagaggaagtaaaagtcgtaacaaggtttccgtaggtgaacctgcggaaggatcattacctagagtttgtggacttcggtctgctacctcttacccatgtcttttgagtaccttcgtttcctcggcgggtccgcccgccggttggacaacattcaaaccctttgcagttgcaatcagcgtctgaaaaaacttaatagttacaactttcaacaacggatctcttggttctggcatcgatgaagaacgcagcgaaatgcgataagtagtgtgaattgcagaattcagtgaatcatcgaatctttgaacgcacattgcgccccttggtattccatggggcatgcctgttcgagcgtcatttgtaccttcaagctctgcttggtgttgggtgttttgtctcgcctccgcgcgcagactcgccttaaaacaattggcagccggcgtattgatttcggagcgcagtacatctcgcgctttgcactcataacgacgacgtccaaaagtacatttttacactcttgacctcggatcaggtagggatacccgctgaacttaagcatatcaataagcggaggaaaagaaaccaacagggattgccctagtaacggcgagtgaagcggcaacagctcaaatttgaaatctggcgtctttggcgtccgagttgtaatttgcagagggcgctttggcattggcagcggtccaagttccttggaacaggacgtcacagagggtgagaatcccgtacgtggtcgctagcctttaccgtgtaaagccccttcgacgagtcgagttgtttgggaatgcagctctaaatgggaggtaaatttcttctaaagctaaatactggccagagaccgatagcgcacaagtagagtgatcgaaagatgaaaagcactttggaaagagagttaaaaagcacgtgaaattgttgaaagggaagcgcttgcagccagacttgcctgtagttgctcatccgggtttctacccggtgcactcttctacgggcaggccagcatcagtttgggcggttggataaaggtctctgtcatgtacctcccttcggggagatcttataggggagacgacatgcaaccagcctggactgaggtccgcgcatctgctaggatgctggcgtaatggctgtaagcggcccgtcttgaaacacggaccaaggagtctaacatctatgcgagtgtttgggtgtcaagcccgagcgcgtaatgaaagtgaacggaggtgggaacctttcggggtgcaccatcgaccgatcctgatgtcttcggatggatttgagtaagagcatagctgttgggacccgaaagatggtgaactatgcttgaatagggtgaagccagaggaaactctggtggaggctcgcagcggttctgacgtgcaaatcgatcgtcaaatttgggcataggggcgaaagac

SEQ ID NO: 48 - DNA, 383 bp, unidentified
  source 1..383: mol_type = other DNA; note = Epicoccum nigrum
ccagactggccgaagacgaagttatcgggacggaagagctggccgaaggggccggcgcggacagcgtccattgtaccgggctccaagtcgacgaggacggcacggggaacgaacttgttgccagaggcctgcgggaggtcagcactcgcagtccgtctcaggaaagcgtgtcgtttctagtacctcgttgaagtagacgttcatgcgctcgagctggaggtccgaggtgccgttgtagacaccggagccgtcgaggccatgctcgccggagatggtctgccagaaggcagcaccgatttggttaccctgtcccttgtgagctgccgtccatgagagaacatgcaagtggtgtacttacgcactgaccggtctggaggtgaacc

SEQ ID NO: 49 - DNA, 608 bp, unidentified
  source 1..608: mol_type = other DNA; note = Epicoccum nigrum
ctcctcctcctcctcctgatcgaactcgccccccaccaactccaccagcccacccagccgcccaaaaacctccccatccccgctagccgcccccatggcccggacaagcagcnnnnnnnnnnnnnngtccgggatggccttaggttcgctctcgagctgctccagccgcgacaggacatgcagcagctccctgcgcagcgagtccggcgccggcatgcggccccgcagcggctggtcggcgatgtatgtcttgagcgggagcgcggcgcgcaggatgaggtagggcgccgctgcctgggcgagtttgatgtgcgagtgcgagggcgagggcgaggtactggcggggcgtgcggccacgagggcgaagagggaggccgtgcaggtgtagctcatctttgtgcgcaaggtggcggggaggacggccgtctggccggtgcggacggagaggaggttggcgaggggcgaggtggtgatgggagggagttctccttgtacgggagtgtggatgagggaggtggtgaagaggttgcgggcgtatgtcctgcggagggtgtcggggagggagggggagtttagagggccgctgatcaagtcgcgtagctctgtga

SEQ ID NO: 50 - DNA, 485 bp, unidentified
  source 1..485: mol_type = other DNA; note = Epicoccum nigrum
gacttcggtctgctacctcttacccatgtcttttgagtaccttcgtttcctcggcgggtccgcccgccggttggacaacattcaaaccctttgcagttgcaatcagcgtctgaaaaaacttaatagttacaactttcaacaacggatctcttggttctggcatcgatgaagaacgcagcgaaatgcgataagtagtgtgaattgcagaattcagtgaatcatcgaatctttgaacgcacattgcgccccttggtattccatggggcatgcctgttcgagcgtcatttgtaccttcaagctctgcttggtgttgggtgttttgtctcgcctccgcgcgcagactcgccttaaaacaattggcagccggcgtattgatttcggagcgcagtacatctcgcgctttgcactcataacgacgacgtccaaaagtacatttttacactcttgacctcggatcaggtagggatacccgctgaacttaagca

SEQ ID NO: 51 - DNA, 487 bp, unidentified
  source 1..487: mol_type = other DNA; note = Epicoccum nigrum
ggacttcggtctgctacctcttacccatgtcttttgagtaccttcgtttcctcggcgggtccgcccgccggttggacaacattcaaaccctttgcagttgcaatcagcgtctgaaaaaacttaatagttacaactttcaacaacggatctcttggttctggcatcgatgaagaacgcagcgaaatgcgataagtagtgtgaattgcagaattcagtgaatcatcgaatctttgaacgcacattgcgccccttggtattccatggggcatgcctgttcgagcgtcatttgtaccttcaagctctgcttggtgttgggtgttttgtctcgcctccgcgcgcagactcgccttaaaacaattggcagccggcgtattgatttcggagcgcagtacatctcgcgctttgcactcataacgacgacgtccaaaagtacatttttacactcttgacctcggatcaggtagggatacccgctgaacttaagcat

SEQ ID NO: 52 - DNA, 490 bp, unidentified
  source 1..490: mol_type = other DNA; note = Epicoccum nigrum
ggacttcggtctgctacctcttacccatgtcttttgagtaccttcgtttcctcggcgggtccgcccgccggttggacaacattcaaaccctttgcagttgcaatcagcgtctgaaaaaacttaatagttacaactttcaacaacggatctcttggttctggcatcgatgaagaacgcagcgaaatgcgataagtagtgtgaattgcagaattcagtgaatcatcgaatctttgaacgcacattgcgccccttggtattccatggggcatgcctgttcgagcgtcatttgtaccttcaagctctgcttggtgttgggtgttttgtctcgcctccgcgcgcagactcgccttaaaacaattggcagccggcgtattgatttcggagcgcagtacatctcgcgctttgcactcataacgacgacgtccaaaagtacatttttacactcttgacctcggatcaggtagggatacccgctgaacttaagcatatc

SEQ ID NO: 53 - DNA, 1182 bp, unidentified
  source 1..1182: mol_type = other DNA; note = Epicoccum nigrum
cccatggcttgcttacccatagcagactggtatgtgttacgaggcgactggttgtgatccgggaaaggaatgatactggcacagataccgagaatcatagcagggtggatctcgcaatgcgtgtaggcataaatgcgaggatccggcttaggcttgatacgcgcgagacggtctttgccctgtggattgcgagcatcttgtgggatacccattttcatgtcacgccactcgccaagatcttcgggcgacatgacaatcatggcagtctcctcctcttccgcgtccaggtattcaataacaccatcttgaatgaggcccttccaaccataaggttctggcatcccctcttcctcgctgaagcctgcatcctgctcttgattgagctggttgcagtggtctcggttgtagacgaggtggttcctgttaggtttgcgcacgtcgttctccactacgaagagaggcctcataacacgtccagcatctgtgaagattttgaactctcggtcacggatgtctcgaatcagactcatctcgtaagagagggttccattacggcgaagctcctgcactgtagagacgagctgctgtgcgttggagtgcacaccgacccatacaccgttaacgaagaccttggtggcttcggggttttgagcttgatcatattcttcaaggagcagcatgttccgctgacccatgaaatcggatataggtccggcatcgctaccgacactgacgtagcacatcagggacaagttcttgacaagaccacaggcctgcccttcaggtgtctcggcggggcacacaagaccccaatgactgttgtgtaactgacggggctttgcgagcttgccgtcacggccgaccggggtgtttgttcgtcgcaaatgcgacaatgtggatgcgtatgtgtaacggttcaagacctgtgagacaccagctttcgccgaagcagccttcttctgatcaccccagtttcccgtggcgagcgaatatttcaaaccattagtaatgacactagctcggacggccatctgcacgttgaagtcggaattggtctcgacgcacttctggaggtacttgtaaacgtccttggttaacttcaggaagagaacgcgaaacaagttcgcaatcagcggcccagcgagatcgagacgcttcttaccaaagtggtcacgatcgtcc

SEQ ID NO: 54 - DNA, 1008 bp, unidentified
  source 1..1008: mol_type = other DNA; note = Epicoccum nigrum
gtcgacttcaacgtccctctcgacgcagacaagaacatcaccaacccccagcgcatcgtcggtgctctccccacaatcaagtatgccgtcgagaacggagctaaggccgtcatccttatgtcccacctcggccgcccggatggcaagccgaacccgaagtacagtctcaagccggttgtttccgagcttgagaagcttctcggcaagagcgtgactttcacagatgactgcgtcggcaagcaggtcgaggagaccgtcaacaatgccaaggacggacaggtcatccttcttgagaacctccgcttccacgctgaggaggagggcagctacaaggacgaggagggcaagaagcagaaggtcgacaaggccaaggttgaggagttccgcaagggactgactgctcttggtgatgtttacatcagtgagtacggccgtctgaggtcgcagttcctgactaatatatccgcagacgatgctttcggcacagctcaccgtgctcacagctccatggtcggcgttgacttgccccagaaggcttccggtttcctcgtaaagaaggagctcgagtactttgctaaggcgctcgaggagcccaagaggcccttcctcgccatcctcggtggcgccaaggtctccgataagatccagctcatcgacaacctgctcggcaaggtcgacagcttgattatctgtggcggtatgtcattcacattcaagaagaccctcgagggcgtcagcatcggtgactctctgttcgacgaggctggcagcaagactgtcaaggaccttgttgagaaggctaagaaaaacaacgtcaagattgtcctgcctgttgactacatcaccgccgacaagttcgacaaggacgccaagacagggtacgctgaggacaaggacggtattccagatggctggatgggtcttgactgcggcaagaagtcgatcgagctttacaaggaggccatcggcgaggctcagaccatcctgtggaacggtcccgctggtgtc

SEQ ID NO: 55 - DNA, 1182 bp, unidentified; identical to SEQ ID NO: 53
  source 1..1182: mol_type = other DNA; note = Epicoccum nigrum

SEQ ID NO: 56 - DNA, 400 bp, unidentified
  source 1..400: mol_type = other DNA; note = Epicoccum nigrum
cttcagctggaacttgatggtggacttgacgtcggtcatcttgttggcgaggtcctcagcgtgagagacgggagtggggaactttccagccttggacagaccaggacccaagagacgggggatctgcttgatcagggcgtcggaagccatgaaagcatcgtacttgcgtgcaagcttcttgatcagcttcttgttcttgttgagcttcttcaagtcctcagtggacatggcatcaacaccgtggtgcttggcacgatcgatatcgtgctggtcaccgagaacgcagatggccatgcctgcacaactgtcagctcttgctactgcaatccttgggcgcgcgcaacgtacgaggacgggggacggtggggagcttgacggttccagagaaacgcttgtcacg

SEQ ID NO: 57 - DNA, 489 bp, unidentified
  source 1..489: mol_type = other DNA; note = Epicoccum nigrum
ggtgaatcgcacatgctagatttccacattctgaagaatatcccagccaacgttattttgagcgaagacttccttctgggagacgagaccaacgctttcgcagactacgcgtgctacatgcacgatgaggaggaggacgacgacgatgctagttgcttcatgattgacatcgactattcgtacggcaatccggccgtcgacacccgagagtatcgggacaaccttgagaccgtgcgctacggagaggaagaggactggatcgacagtctatctggcgacgaaaagagaggtgcccaagcagctgtgcaactaagacgcgcgcagtgggatccaacacaacccatgactggcgcgaacgccatgaacgtggttcctccgcagttccggcaggcaagtagtcatccgaagactgtgcgcaagcgtttgaagtttcgactgtggacgagaaaaaagactgtctagtgcagtctcatacgctctgtctggtcg

SEQ ID NO: 58 - DNA, 490 bp, unidentified
  source 1..490: mol_type = other DNA; note = Epicoccum nigrum
gtagacttcggtctgctacctcttacccatgtcttttgagtaccttcgtttcctcggcgggtccgcccgccgattggacaacattcaaaccctttgcagttgcaatcagcgtctgaaaaaacataatagttacaactttcaacaacggatctcttggttctggcatcgatgaagaacgcagcgaaatgcgataagtagtgtgaattgcagaattcagtgaatcatcgaatctttgaacgcacattgcgccccttggtattccatggggcatgcctgttcgagcgtcatttgtaccttcaagctctgcttggtgttgggtgtttgtctcgcctctgcgtgtagactcgccttaaaacaattggcagccggcgtattgatttcggagcgcagtacatctcgcgctttgcactcataacgacgacgtccaaaagtacatttttacactcttgacctcggatcaggtagggatacccgctgaacttaagcatat

SEQ ID NO: 59 - DNA, 485 bp, unidentified
  source 1..485: mol_type = other DNA; note = Epicoccum nigrum
agacttcggtctgctacctcttacccatgtcttttgagtaccttcgtttcctcggcgggtccgcccgccgattggacaacattcaaaccctttgcagttgcaatcagcgtctgaaaaaacataatagttacaactttcaacaacggatctcttggttctggcatcgatgaagaacgcagcgaaatgcgataagtagtgtgaattgcagaattcagtgaatcatcgaatctttgaacgcacattgcgccccttggtattccatggggcatgcctgttcgagcgtcatttgtaccttcaagctctgcttggtgttgggtgtttgtctcgcctctgcgtgtagactcgccttaaaacaattggcagccggcgtattgatttcggagcgcagtacatctcgcgctttgcactcataacgacgacgtccaaaagtacatttttacactcttgacctcggatcaggtagggatacccgctgaacttaagca

SEQ ID NO: 60 - DNA, 489 bp, unidentified
  source 1..489: mol_type = other DNA; note = Epicoccum nigrum
gtggacttcggtctgctacctcttacccatgtcttttgagtaccttcgtttcctcggcgggtccgcccgccggttggacaacattcaaaccctttgcagttgcaatcagcgtctgaaaaaacttaatagttacaactttcaacaacggatctcttggttctggcatcgatgaagaacgcagcgaaatgcgataagtagtgtgaattgcagaattcagtgaatcatcgaatctttgaacgcacattgcgccccttggtattccatggggcatgcctgttcgagcgtcatttgtaccttcaagctctgcttggtgttgggtgttttgtctcgcctccgcgcgcagactcgccttaaaacaattggcagccggcgtattgatttcggagcgcagtacatctcgcgctttgcactcataacgacgacgtccaaaagtacatttttacactcttgacctcggatcaggtagggatacccgctgaacttaagcat

SEQ ID NO: 61 - DNA, 484 bp, unidentified
  misc_feature 1..484: note = Kingdom: Fungi, Phylum: Ascomycota, Class: Dothideomycetes, Order: Pleosporales, Family: Didymellaceae
  source 1..484: mol_type = other DNA
cttcggtctgctacctcttacccatgtcttttgagtaccttcgtttcctcggcgggtccgcccgccgattggacaacattcaaaccctttgcagttgcaatcagcgtctgaaaaaacataatagttacaactttcaacaacggatctcttggttctggcatcgatgaagaacgcagcgaaatgcgataagtagtgtgaattgcagaattcagtgaatcatcgaatctttgaacgcacattgcgccccttggtattccatggggcatgcctgttcgagcgtcatttgtaccttcaagctctgcttggtgttgggtgtttgtctcgcctctgcgtgtagactcgccttaaaacaattggcagccggcgtattgatttcggagcgcagtacatctcgcgctttgcactcataacgacgacgtccaaaagtacatttttacactcttgacctcggatcaggtagggatacccgctgaacttaagcata

SEQ ID NO: 62 - DNA, 533 bp, unidentified
  misc_feature 1..533: note = Kingdom: Fungi, Phylum: Ascomycota, Class: Dothideomycetes, Order: Pleosporales, Family: Massarinaceae, Genus: Stagonospora
  source 1..533: mol_type = other DNA
agctactggcatggggactgttagtctgcatggtatcactaccgatgagcagcaggtcccctgtctatacccttgttttttgcgtacctattgtttcctcggcgggcttgctcgccggctggacaaaatctataacctttttttaatcttcaatcagcgtctgaaattatacataataattacaactttcaacaacggatctcttggttctggcatcgatgaagaacgcagcgaaatgcgataagtagtgtgaattgcagaattcagtgaatcatcgaatctttgaacgcacattgcgccccttggtattccatggggcatgcctgttcgagcgtcatttgtaccctcaagctttgcttggtgttgggcgtcttgtcttttatcagactcgccttaaagtaattggcagccagtgtttttggtagtaagcgcagcacaatttgcgtcttggtccctaaacagtggcatccacaaagcctctttctcacttttgacctcggatcaggtagggatacccgctgaacttaagcata

SEQ ID NO: 63 - DNA, 508 bp, unidentified
  source 1..508: mol_type = other DNA; note = Alternaria eichhorniae
gggctggacctctcggggttacagccttgctgaattattcacccttgtcttttgcgtacttcttgtttccttggtgggttcgcccaccactaggacaaacataaaccttttgtaattgcaatcagcgtcagtaacaaattaataattacaactttcaacaacggatctcttggttctggcatcgatgaagaacgcagcgaaatgcgataagtagtgtgaattgcagaattcagtgaatcatcgaatctttgaacgcacattgcgccctttggtattccaaagggcatgcctgttcgagcgtcatttgtaccctcaagctttgcttggtgttgggcgtcttgtctctagctttgctggagactcgccttaaagtaattggcagccggcctactggtttcggagcgcagcacaagtcgcactctctatcagcaaaggtctagcatccattaagcctttttttcaacttttgacctcggatcaggtagggatacccgctgaacttaagcat

SEQ ID NO: 64 - DNA, 513 bp, unidentified
  source 1..513: mol_type = other DNA; note = Alternaria eichhorniae
gcgggctggacctctcggggttacagccttgctgaattattcacccttgtcttttgcgtacttcttgtttccttggtgggttcgcccaccactaggacaaacataaaccttttgtaattgcaatcagcgtcagtaacaaattaataattacaactttcaacaacggatctcttggttctggcatcgatgaagaacgcagcgaaatgcgataagtagtgtgaattgcagaattcagtgaatcatcgaatctttgaacgcacattgcgccctttggtattccaaagggcatgcctgttcgagcgtcatttgtaccctcaagctttgcttggtgttgggcgtcttgtctctagctttgctggagactcgccttaaagtaattggcagccggcctactggtttcggagcgcagcacaagtcgcactctctatcagcaaaggtctagcatccattaagcctttttttcaacttttgacctcggatcaggtagggatacccgctgaacttaagcatatc

SEQ ID NO: 65 - DNA, 509 bp, unidentified
  source 1..509: mol_type = other DNA; note = Alternaria eichhorniae
gggctggacctctcggggttacagccttgctgaattattcacccttgtcttttgcgtacttcttgtttccttggtgggttcgcccaccactaggacaaacataaaccttttgtaattgcaatcagcgtcagtaacaaattaataattacaactttcaacaacggatctcttggttctggcatcgatgaagaacgcagcgaaatgcgataagtagtgtgaattgcagaattcagtgaatcatcgaatctttgaacgcacattgcgccctttggtattccaaagggcatgcctgttcgagcgtcatttgtaccctcaagctttgcttggtgttgggcgtcttgtctctagctttgctggagactcgccttaaagtaattggcagccggcctactggtttcggagcgcagcacaagtcgcactctctatcagcaaaggtctagcatccattaagccttttttttcaacttttgacctcggatcaggtagggatacccgctgaacttaagcat

SEQ ID NO: 66 - DNA, 497 bp, unidentified
  source 1..497: mol_type = other DNA; note = Alternaria eichhorniae
ctcggggttacagccttgctgaattattcacccttgtcttttgcgtacttcttgtttccttggtgggttcgcccaccactaggacaaacataaaccttttgtaattgcaatcagcgtcagtaacaaattaataattacaactttcaacaacggatctcttggttctggcatcgatgaagaacgcagcgaaatgcgataagtagtgtgaattgcagaattcagtgaatcatcgaatctttgaacgcacattgcgccctttggtattccaaagggcatgcctgttcgagcgtcatttgtaccctcaagctttgcttggtgttgggcgtcttgtctctagctttgctggagactcgccttaaagtaattggcagccggcctactggtttcggagcgcagcacaagtcgcactctctatcagcaaaggtctagcatccattaagcctttttttcaacttttgacctcggatcaggtagggatacccgctgaacttaagcat

SEQ ID NO: 67 - DNA, 508 bp, unidentified
  source 1..508: mol_type = other DNA; note = Alternaria planifunda
cgggctggcatccttcggggttacagccttgctgaattattcacccgtgtcttttgcgtacttcttgtttccttggtgggttcgcccaccataggacaaaccataaaccttttgtaattgcaatcagcgtcagtaaaaaaattaataattacaacttttaacaacggatctcttggttctggcatcgatgaagaacgcagcgaaatgcgataagtagtgtgaattgcagaattcagtgaatcatcgaatctttgaacgcacattgcgccctttggtattccaaagggcatgcctgttcgagcgtcatttgtaccctcaagctttgcttggtgttgggcgtcttgtctccagttcgctggagactcgccttaaagtaattggcagccggcctactggtttcggagcgcagcacaagtcgcgctctcttccagccaaggtcagcatccacaaagccttttttcaacttttgacctcggatcaggtagggatacccgctgaacttaagcat

SEQ ID NO: 68 - DNA, 507 bp, unidentified
  source 1..507: mol_type = other DNA; note = Bipolaris spicifera
gccgttcgcggctggactatttattacccttgtcttttgcgcacttgttgtttcctgggcgggttcgctcgccaccaggaccacaatataaaccttttttatgcagttgcaatcagcgtcagtataacaaatgtaaatcatttacaactttcaacaacggatctcttggttctggcatcgatgaagaacgcagcgaaatgcgatacgtagtgtgaattgcagaattcagtgaatcatcgaatctttgaacgcacattgcgccctttggtattccaaagggcatgcctgttcgagcgtcatttgtaccctcaagctttgcttggtgttgggcgtttttgtctttggcccgccaaagactcgccttaaaatgattggcagccggcctactggtttcgcagcgcagcacatttttgcgcttgcaatcagcaaaagaggacggcaatccatcaagactccttctcacgtttgacctcggatcaggtagggatacccgctgaacttaagcat

SEQ ID NO: 69 - DNA, 498 bp, unidentified
  source 1..498: mol_type = other DNA; note = Bipolaris spicifera
ttcgcggctggactatttattacccttgtcttttgcgcacttgttgtttcctgggcgggttcgctcgccaccaggaccacaatataaaccttttttatgcagttgcaatcagcgtcagtataacaaatgtaaatcatttacaactttcaacaacggatctcttggttctggcatcgatgaagaacgcagcgaaatgcgatacgtagtgtgaattgcagaattcagtgaatcatcgaatctttgaacgcacattgcgccctttggtattccaaagggcatgcctgttcgagcgtcatttgtaccctcaagctttgcttggtgttgggcgtttttgtctttggcccgccaaagactcgccttaaaatgattggcagccggcctactggtttcgcagcgcagcacatttttgcgcttgcaatcagcaaaagaggacggcaatccatcaagactccttctcacgtttgacctcggatcaggtagggatacccgctgaactta

SEQ ID NO: 70 - DNA, 511 bp, unidentified
  source 1..511: mol_type = other DNA; note = Stemphylium herbarum
ggacctcacctcggtgagggctccagcttgtctgaattattcacccatgtcttttgcgcacttcttgtttcctgggcgggttcgcccgccaccaggaccaaaccataaacctttttgtaattgcaatcagcgtcagtaaacaatgtaattattacaactttcaacaacggatctcttggttctggcatcgatgaagaacgcagcgaaatgcgatacgtagtgtgaattgcagaattcagtgaatcatcgaatctttgaacgcacattgcgccctttggtattccaaagggcatgcctgttcgagcgtcatttgtaccctcaagctttgcttggtgttgggcgtctttgtctctcacgagactcgccttaaaatgattggcagccgacctactggtttcggagcgcagcacaattcttgcactttgaatcagccttggttgagcatccatcaagaccacatttttttcaacttttgacctcggatcaggtagggatacccgctgaacttaag

SEQ ID NO: 71 - DNA, 513 bp, unidentified
  source 1..513: mol_type = other DNA; note = Stemphylium herbarum
ggacctcacttcggtgcgggctccagcttgtctgaattattcacccatgtcttttgcgcacttcttgtttcctgggcgggttcgcccgccaccaggaccaaaccataaacctttttgtaattgcaatcagcgtcagtaaacaatgtaattattacaactttcaacaacggatctcttggttctggcatcgatgaagaacgcagcgaaatgcgatacgtagtgtgaattgcagaattcagtgaatcatcgaatctttgaacgcacattgcgccctttggtattccaaagggcatgcctgttcgagcgtcatttgtaccctcaagctttgcttggtgttgggcgtcttttgtctctcacgagactcgccttaaaatgattggcagccgacctactggtttcggagcgcagcacaattcttgcactttgaatcagccttggttgagcatccatcaagaccacattttttaacttttgacctcggatcaggtagggatacccgctgaacttaagcat

SEQ ID NO: 72 - DNA, 509 bp, unidentified
  misc_feature 1..509: note = Kingdom: Fungi, Phylum: Ascomycota, Class: Dothideomycetes, Order: Pleosporales, Family: Pleosporaceae
  source 1..509: mol_type = other DNA
cgatggggcagcccctcttttggggttgccccctctggcgccatattcacccatgtctttttgcgtactacttgtttccttggcgggttcgcccgccatttggacctttactcaacccttttttttttgtgcaattgccatcagcgtcagcaaaacaatgtaatcaatttacaactttcaacaacggatctcttggttctggcatcgatgaagaacgcagcgaaatgcgataagtagtgtgaattgcaaaattcagtgaatcatcgaatctttgaacgcacattgcgccctttggtattccaaagggcatgcctgttcgagcgtcatttgtaccctcaagctttgcttggtgttgggcgtttcttgtcctgggccttgcagcccgggactcgccttaaaatcattggcagccggcctactggtttcggagcgcagcacattttttgcgctcttgtccagccatggtcgtgcatccaaaaggtactttttttttttcacgtttgacctcg

SEQ ID NO: 73 - DNA, 472 bp, unidentified
  source 1..472: mol_type = other DNA; note = Preussia africana
tcgagatagaacccttgcctttttgagtaccttttcgtttcctcggcaggctcgcctgccaatggggaccccaacaaacactttgcagtacctgtaaacagtctgaacaaacttttaaaaattaaaactttcaacaacggatctcttggttctggcatcgatgaagaacgcagcgaaatgcgataagtagtgtgaattgcagaattcagtgaatcatcgaatctttgaacgcacattgcgccctttggtattccttagggcatgcctgttcgagcgtcattgaaaccttcaagctcagcttggtgttgggtgactgtccgcttgcggactcgcctcaaaatgattggcggccggtacttttggcttcgagcgcagcagaaacgcgaactcgaggcctgtgtgctggctcccagaagctatcttcacaattttgacctcggatcaggtagggatacccgctgaacttaagcat

SEQ ID NO: 74 - DNA, 1496 bp, unidentified
  source 1..1496: mol_type = other DNA; note = Curvularia protuberata
tcttggtcatttagaggaagtaaaagtcgtaacaaggtctccgtaggtgaacctgcggagggatcattacacaataacatatgaaggctgtacgccgctgcgcccccgggccagttggctgaggctggattatttattacccttgtcttttgcgcacttgttgtttcctgggcgggttcgcccgcctccaggaccacaccataaaccttttttatgcagttgcaatcagcgtcagtacaacaaatgtaaatcatttacaactttcaacaacggatctcttggttctggcatcgatgaagaacgcagcgaaatgcgatacgtagtgtgaattgcagaattcagtgaatcatcgaatctttgaacgcacattgcgccctttggtattccaaagggcatgcctgttcgagcgtcatttgtaccctcaagctttgcttggtgttgggcgtttttgtctttggtttgccaaagactcgccttaaaacgattggcagccggcctcctggttacgcagcgcagcacatttttgcgcttgcaatcagcaagagggcggcactccatcaagactccttctcacgtttgacctcggatcaggtagggatacccgctgaacttaagcatatcaataagcggaggaaaagaaaccaacagggattgccctagtaacggcgagtgaagcggcaacagctcaaatttgaaatctggctcttttagggtccgagttgtaatttgcagagggcgctttggctttggcagcggtccaagttccttggaacaggacgtcacagagggtgagaatcccgtacgtggtcgctagctattgccgtgtaaagccccttcgacgagtcgagttgtttgggaatgcagctctaaatgggaggtaaatttcttctaaagctaaatattggccagagaccgatagcgcacaagtagagtgatcgaaagatgaaaagcactttggaaagagagtcaaacagcacgtgaaattgttgaaagggaagcgcttgcagccagacttgcttgcagttgctcatccgggcttttgcccggtgcactcttctgtaggcaggccagcatcagtttgggcggtgggataaaggtctctgacacgttccttccttcgggttggccatataggggagacgtcataccaccagcctggactgaggtccgcgcatctgctaggatgctggcgtaatggctgtaagcggcccgtcttgaaacacggaccaaggagtctaacatctatgcgagtgtttgggtgtcaagcccgagcgcgtaatgaaagtgaacggaggtgggaacccgcaagggcgcaccatcgaccgatcctgaagtttacggaaggatttgagtaagagcatggctgttgggacccgaaagatggtgaactatgcttgaatagggtgaagccagaggaaactctggtggaggctcgcagcggttctgacgtgcaaatcgatcgtcaaatttgggcataggggcgaaagactaatcga

SEQ ID NO: 75 - DNA, 1010 bp, unidentified
  source 1..1010: mol_type = other DNA; note = Curvularia protuberata
acaccggcagggccgttccagaggatggtctgcgcctcatcgatggcctccttgtaaagcttgatcgacttctctccacagtcgagacccatccagccatctgggataccatccttgtcctcggcataaccgacgttggcgtccttgtcgaacttgtcggcggtgatgtagtcaacaggcagcacaatcttgacattgttcttcttcgccttctccacgaggtccttgacggtcttgctaccagcctcatcgaacaagctttcaccaatcttgacgccctcgagagtcttcttgaaggtgaaggacatgcctccgcaaatgatcaggctgttgaccttgccaagcaggttgtcgatcaattgaatcttgtcagagaccttggcaccaccaaggatggcgaggaaaggtcgcttggggttctcaagcgcttgtgcaaagtaatcaagctccttcttgacaaggaagccagaggccttttgtgggaggtcgacaccgaccatggagctgtgcgcgcggtgagcagtaccaaaagcgtcgtctgtaaaccgtcagcctcgtgctttcgcccatgaattcatagttacttactaatgtagacgtcgcccagagcagtcagtcccttcctaaactcatcgaccttgctcttgtcgaccttctgcttcttgcccgcatcatccttgtagctaccctcctcctcagcgtggaagcgcaggttctcgaggaggatgacctgaccaccgctagcgttgttgacggtatcctctaccgacttgccgacgcagtcgtcggtgaaggtaacgctcttgccgaggagcttctcgagttcgggaacaaccggcttgaggctgtacttcgcattgggcttaccgtctggccggccaaggtgggacatgagaatgacggccttggcgccattgtcgacggcgtacttgattgtgggaagtgcgccaacaatgcgctggttgttggtgatcttcttgtcggcgtcgagagggacgttgaagtcgacc

SEQ ID NO: 76 - DNA, 1497 bp, unidentified
  source 1..1497: mol_type = other DNA; note = Curvularia protuberata
tccgtaggtgaacctgcggagggatcattacacaataacatatgaaggctgtacgccgctgcgcccccgggccagttggctgaggctggattatttattacccttgtcttttgcgcacttgttgtttcctgggcgggttcgcccgcctccaggaccacaccataaaccttttttatgcagttgcaatcagcgtcagtacaacaaatgtaaatcatttacaactttcaacaacggatctcttggttctggcatcgatgaagaacgcagcgaaatgcgatacgtagtgtgaattgcagaattcagtgaatcatcgaatctttgaacgcacattgcgccctttggtattccaaagggcatgcctgttcgagcgtcatttgtaccctcaagctttgcttggtgttgggcgtttttgtctttggtttgccaaagactcgccttaaaacgattggcagccggcctcctggttacgcagcgcagcacatttttgcgcttgcaatcagcaagagggcggcactccatcaagactccttctcacgtttgacctcggatcaggtagggatacccgctgaacttaagcatatcaataagcggaggaaaagaaaccaacagggattgccctagtaacggcgagtgaagcggcaacagctcaaatttgaaatctggctcttttagggtccgagttgtaatttgcagagggcgctttggctttggcagcggtccaagttccttggaacaggacgtcacagagggtgagaatcccgtacgtggtcgctagctattgccgtgtaaagccccttcgacgagtcgagttgtttgggaatgcagctctaaatgggaggtaaatttcttctaaagctaaatattggccagagaccgatagcgcacaagtagagtgatcgaaagatgaaaagcactttggaaagagagtcaaacagcacgtgaaattgttgaaagggaagcgcttgcagccagacttgcttgcagttgctcatccgggcttttgcccggtgcactcttctgtaggcaggccagcatcagtttgggcggtgggataaaggtctctgacacgttccttccttcgggttggccatataggggagacgtcataccaccagcctggactgaggtccgcgcatctgctaggatgctggcgtaatggctgtaagcggcccgtcttgaaacacggaccaaggagtctaacatctatgcgagtgtttgggtgtcaagcccgagcgcgtaatgaaagtgaacggaggtgggaacccgcaagggcgcaccatcgaccgatcctgaagtttacggaaggatttgagtaagagcatggctgttgggacccgaaagatggtgaactatgcttgaatagggtgaagccagaggaaactctggtggaggctcgcagcggttctgacgtgcaaatcgatcgtcaaatttgggcataggggcgaaagactaatcgaactatctagtagctggttcctgccgaagtttccctcagga

SEQ ID NO: 77 - DNA, 1472 bp, unidentified
  source 1..1472: mol_type = other DNA; note = Curvularia spicifera
tccgtaggtgaacctgcggagggatcattacacaataaaatacgaaggccgttcgcggctggactatttattacccttgtcttttgcgcacttgttgtttcctgggcgggttcgctcgccaccaggaccacaatataaaccttttttatgcagttgcaatcagcgtcagtataacaaatgtaaatcatttacaactttcaacaacggatctcttggttctggcatcgatgaagaacgcagcgaaatgcgatacgtagtgtgaattgcagaattcagtgaatcatcgaatctttgaacgcacattgcgccctttggtattccaaagggcatgcctgttcgagcgtcatttgtaccctcaagctttgcttggtgttgggcgtttttgtctttggcccgccaaagactcgccttaaaatgattggcagccggcctactggtttcgcagcgcagcacatttttgcgcttgcaatcagcaaaagaggacggcaatccatcaagactccttctcacgtttgacctcggatcaggtagggatacccgctgaacttaagcatatcaataagcggaggaaaagaaaccaacagggattgccctagtaacggcgagtgaagcggcaacagctcaaatttgaaatctggctctttcagagtccgagttgtaatttgcagagggcgctttggctttggcagcggtccaagttccttggaacaggacgtcacagagggtgagaatcccgtacgtggtcgctagctattgccgtgtaaagccccttcgacgagtcgagttgtttgggaatgcagctctaaatgggaggtaaatttcttctaaagctaaatattggccagagaccgatagcgcacaagtagagtgatcgaaagatgaaaagcactttggaaagagagtcaaacagcacgtgaaattgttgaaagggaagcgcttgcagccagacttgcttgcagttgctcatccgggcttttgcccggtgcactcttctgcaggcaggccagcatcagtttgggcggtgggataaaggtctctgtcacgtaccttccttcgggttggccttataggggagacgccataccaccagcctggactgaggtccgcgcatctgctaggatgctggcgtaatggctgtaagcggcccgtcttgaaacacggaccaaggagtctaacatctatgcgagtgtttgggtgtcaagcccgagcgcgtaatgaaagtgaacggaggtgggaacccgcaagggtgcaccatcgaccgatcctgaagtttacggaaggatttgagtaagagcatggctgttgggacccgaaagatggtgaactatgcttgaatagggtgaagccagaggaaactctggtggaggctcgcagcggttctgacgtgcaaatcgatcgtcaaatttgggcataggggcgaaagactaatcgaactatctagtagctggttcctgccgaagtttccctcagga

SEQ ID NO: 78 - DNA, 1428 bp, unidentified
  source 1..1428: mol_type = other DNA; note = Curvularia spicifera
aggtgaacctgcggagggatcattacacaataaaatacgaaggccgttcgcggctggactatttattacccttgtcttttgcgcacttgttgtttcctgggcgggttcgctcgccaccaggaccacaatataaaccttttttatgcagttgcaatcagcgtcagtataacaaatgtaaatcatttacaactttcaacaacggatctcttggttctggcatcgatgaagaacgcagcgaaatgcgatacgtagtgtgaattgcagaattcagtgaatcatcgaatctttgaacgcacattgcgccctttggtattccaaagggcatgcctgttcgagcgtcatttgtaccctcaagctttgcttggtgttgggcgtttttgtctttggcccgccaaagactcgccttaaaatgattggcagccggcctactggtttcgcagcgcagcacatttttgcgcttgcaatcagcaaaagaggacggcaatccatcaagactccttctcacgtttgacctcggatcaggtagggatacccgctgaacttaagcatatcaataagcggaggaaaagaaaccaacagggattgccctagtaacggcgagtgaagcggcaacagctcaaatttgaaatctggctctttcagagtccgagttgtaatttgcagagggcgctttggctttggcagcggtccaagttccttggaacaggacgtcacagagggtgagaatcccgtacgtggtcgctagctattgccgtgtaaagccccttcgacgagtcgagttgtttgggaatgcagctctaaatgggaggtaaatttcttctaaagctaaatattggccagagaccgatagcgcacaagtagagtgatcgaaagatgaaaagcactttggaaagagagtcaaacagcacgtgaaattgttgaaagggaagcgcttgcagccagacttgcttgcagttgctcatccgggcttttgcccggtgcactcttctgcaggcaggccagcatcagtttgggcggtgggataaaggtctctgtcacgtaccttccttcgggttggccttataggggagacgccataccaccagcctggactgaggtccgcgcatctgctaggatgctggcgtaatggctgtaagcggcccgtcttgaaacacggaccaaggagtctaacatctatgcgagtgtttgggtgtcaagcccgagcgcgtaatgaaagtgaacggaggtgggaacccgcaagggtgcaccatcgaccgatcctgaagtttacggaaggatttgagtaagagcatggctgttgggacccgaaagatggtgaactatgcttgaatagggtgaagccagaggaaactctggtggaggctcgcagcggttctgacgtgcaaatcgatcgtcaaatttgggcataggggcgaaagactaatcgaa

SEQ ID NO: 79 - DNA, 1197 bp, unidentified
  source 1..1197: mol_type = other DNA; note = Curvularia spicifera
cccatagcttgcttacccatagcagattggtatgtgttacggggcgactggttgtgatctgggaagggaatgatactggcgcaaatacccaagatcatagctgggtgaatctcacaatgggtgtaggcgtggatgcgaggatccggtagtggcttgagacggcggagtcgatccttgccttcagtagatcgctcagctgcaggcaagcccatcttcatttctcgccattcttccaagtcctcgggagagaatgttatcattgcagtttcttcttcctcggcatcgaggtattcaacgacaccgtcttgaatgagacctctccagccgtatgtagcctgctcgacttcctcttgactccagccttgtcttgtactggtctcttgctgttcagccttgagcttgttgctgatttccttggtaaagatgaggtggttccggtttggctttcgaatatcgttttctacaacgaacaaaggcctcatgacacgacccgcatctgtgaatatcttgaactctctgtcgcgaatatcacgaatcaaactcatctcgtaagacagagtaccattgcggcgaagctcctgcacgactgtgacaagctgctgagcatttgaatgaacaccaacccagacaccgttaacgaagaccttggtcgcatccgggttctggttctggtcgtactcctcgagaagttgcatgttacgttgtgtcatgaagtcgataatgggcgatgcatcgctaccaacactgacataacacataagagacaagttcttgaccagaccgcaagcctgtccttcgggcgtctcagcagggcagacaagaccccaatgagagttgtgaagctgtcgcggctttgccaacttaccatcacgtccaacgggggtgttcgttcgacgcagatgggatagtgtggaggcataggtgtatcggttcaacacctgcgaaacaccagccttggcagatgcagccttcttctgatcaccccaattgcctgtagccagagagtacttcaggccgtttgtgatgatgctggctttcacagccatttgaacattgaagtcttggttgttttccacgcaccgctggaggtacttgtagacatccttggtgagcttcaggaacaagattcggaacaagttggcaatcagaggtccagccagatctagtcgcttctttccaaagtgatcacgatcgtcc

SEQ ID NO: 80 - DNA, 387 bp, unidentified
  source 1..387: mol_type = other DNA; note = Curvularia spicifera
gttcacctccagaccggtcaatgcgtaagtctcgcgccgcctgaaaacaccacgggaacgactgctaacagccgctacagggtaaccaaattggtgccgccttctggcagaccatttccggcgagcatggcctcgacggctctggtgtctacaacggcacctctgaccttcagctcgagcgcatgaacgtctacttcaacgaagtacgtccctcggtgaagctccaacagacaaaagaccaatactgatgtgcagcaggcttccaacaacaagttcgtgccccgtgccgtcctcgtcgatctcgagcctggtactatggacgctgtccgcgctggtccctttggtcagctcttccgccccgacaacttcgtcttcggccagtcgggt

SEQ ID NO: 81 - DNA, 590 bp, unidentified
  source 1..590: mol_type = other DNA; note = Curvularia spicifera
ctaccgcaagagcaactgtgcaagtccagcttcagtccctttcgatccccctcgcccaggaagagattcacccacgagcgcaaccagtccgaggcggtccagcgtccacgtcctatgagcgtttgcagcaactcgccagcagttcagcacaccaaaagagcctccatctacgtctccgacgcttccatcatcctagcgcaacgctcacccatggcttctccggtttccccaccagactccatgtcctcccccatccatgaatcgtctgatgccgtcgaccactatgctatcctggagatcacccctagagcaactaccgatgaggtcaaggctgcctaccgccgactacgggtcgtctacttctcaagtgacgcgaagaagtaccgagcactgcaggcggccttcgacgtcttgatggacccgcaatcccgcgaagcttacgacgcaacctatcaaccaactgccgcagcaccagtatcgctcgctagcattggtgagatcctggactcggggaagctatggcgacaggacagcgcccacggagacgacccagtaatcccagaagaggaagaggaggaggaggaggaagt

SEQ ID NO: 82 - DNA, 491 bp, unidentified
  misc_feature 1..491: note = Kingdom: Fungi, Phylum: Ascomycota, Class: Eurotiomycetes, Order: Eurotiales, Family: Aspergillaceae, Genus: Penicillium
  source 1..491: mol_type = other DNA
cggggcccaacctcccacccgtgttgcccgaacctatgttgcctcggcgggccccgcgcccgccgacggcccccctgaacgctgtctgaagttgcagtctgagacctataacgaaattagttaaaactttcaacaacggatctcttggttccggcatcgatgaagaacgcagcgaaatgcgataactaatgtgaattgcagaattcagtgaatcatcgagtctttgaacgcacattgcgccctctggtattccggagggcatgcctgtccgagcgtcattgctgccctcaagcccggcttgtgtgttgggccccgtcccccccgccggggggacgggcccgaaaggcagcggcggcaccgcgtccggtcctcgagcgtatggggcttcgtcacccgctctagtaggcccggccggcgccagccgacccccaacctttaattatctcaggttgacctcggatcaggtagggatacccgctgaacttaagcat

SEQ ID NO: 83 - DNA, 167 bp, unidentified
  source 1..167: mol_type = other DNA; note = Phomopsis liquidambari
tataccttactgttgcctcggcgcatgctggccccctcggggtcccctggagacagggagcaggcacgccggcggccaagttaactcttgtttttacactgaaactctgagaaaaaaacacaaatgaatcaaaactttcaacaacggatctcttggttctggcatcg

SEQ ID NO: 84 - DNA, 482 bp, unidentified
  source 1..482: mol_type = other DNA; note = Gibellulopsis nigrescens
acctgttgcttcggcggcgcgcctctcggggcgtgcccgccggcattatcagaatctctgttcgaacccgacgatacttctgagtgttctaagcgaactgttaaaactttcaacaacggatctcttggctccagcatcgatgaagaacgcagcgaaacgcgatatgtaatgtgaattgcagaattcagtgaatcatcgaatctttgaacgcacatggcgccttccagtatcctgggaggcatgcctgtccgagcgtcgtttcaaccctcgagcccccgtggcccggcgttggggacctgcccaggcagtccccgaaaaccagtggcggacccgacgggcccttcctttgcgtagtaacatctgcctcgcatcgggagcccccgggctatccggcctctaaacccccctcaagcccgctccggcggcaccaaggttgacctcggatcaggtaggaatacccgctgaacttaagcatatcaata

SEQ ID NO: 85 - DNA, 507 bp, unidentified
  source 1..507: mol_type = other DNA; note = Gibellulopsis nigrescens
cctttgtgaccttcatacctgttgcttcggcggcgcgcctctcggggcgtgcccgccggcattatcagaatctctgttcgaacccgacgatacttctgagtgttctaagcgaactgttaaaactttcaacaacggatctcttggctccagcatcgatgaagaacgcagcgaaacgcgatatgtaatgtgaattgcagaattcagtgaatcatcgaatctttgaacgcacatggcgccttccagtatcctgggaggcatgcctgtccgagcgtcgtttcaaccctcgagcccccgtggcccggcgttggggacctgcccaggcagtccccgaaaaccagtggcggacccgacgggcccttcctttgcgtagtaacatctgcctcgcatcgggagcccccgggctatccggcctctaaacccccctcaagcccgctccggcggcaccaaggttgacctcggatcaggtaggaatacccgctgaacttaagcatatcaataagcggagga

SEQ ID NO: 86 - DNA, 511 bp, unidentified
  source 1..511: mol_type = other DNA; note = Gibellulopsis nigrescens
taccctttgtgaccttcatacctgttgcttcggcggcgcgcctctcggggcgtgcccgccggcattatcagaatctctgttcgaacccgacgatacttctgagtgttctaagcgaactgttaaaactttcaacaacggatctcttggctccagcatcgatgaagaacgcagcgaaacgcgatatgtaatgtgaattgcagaattcagtgaatcatcgaatctttgaacgcacatggcgccttccagtatcctgggaggcatgcctgtccgagcgtcgtttcaaccctcgagcccccgtggcccggcgttggggacctgcccaggcagtccccgaaaaccagtggcggacccgacgggcccttcctttgcgtagtaacatctgcctcgcatcgggagcccccgggctatccggcctctaaacccccctcaagcccgctccggcggcaccaaggttgacctcggatcaggtaggaatacccgctgaacttaagcatatcaataagcggaggaa

SEQ ID NO: 87 - DNA, 1458 bp, unidentified
  source 1..1458: mol_type = other DNA; note = Acremonium alternatum
tcttggtcatttagaggaagtaaaagtcgtaacaaggtctccgttggtgaaccagcggagggatcattaccgagtgtaaaaactcccaaaccattgtgaacttaccactgttgcttcggcggcctcgccccgggcgcgttcgcgcggcccggacccaggcgtccgccggaggctccaaactcttgtcttttagtgtatttctgagtggcataagcaaataaatcaaaactttcagcaacggatctcttggttctggcatcgatgaagaacgcagcaaaatgcgataagtaatgtgaattgcagaattcagtgaatcatcgaatctttgaacgcacattgcgcccgccagtattctggcgggcatgcctgtctgagcgtcatttcaaccctcaggacccgttcgcgggacctggcgttggggatcagcctgcccctggcggcggctggccctgaaatccagtggcggttccctcgcgaactcctccgtgcagtaattaaacctctcgcggcaggatagcggttgaaccacgccgttaaaccccccacttctcaaggttgacctcagatcaggtaggaatacccgctgaacttaagcatatcaataagcggaggaaaagaaaccaacagggattgccctagtaacggcgagtgaagcggcaacagctcaaatttgaaatctggcctcacggtccgaattgtaatttgtagaggatgtttctggcgacgtgtcttccgagttccctggaacgggacgccatagagggtgagagccccgtccggtcgtacacctagcctctgtgaaactccttcgacgagtcgagtagtttgggaatgctgctctaaatgggaggtatacgtcttctaaagctaaataccggccagagaccgatagcgcacaagtagagtgatcgaaagatgaaaagcactttgaaaagagggttaaatagtacgtgaaattgctgaaagggaagcgcttatgaccagacttgggctcggtgaatcatccggcgttctcgccggtgcactttgccgtcccaggccagcatcagttcgcgccgggggataaaggtttcgggaatgtagctccttcgggagtgttatagcccgttgcgtaataccctggcgtggactgaggtccgcgctctgcaaggatgctggcgtaatggtcatcagtgacccgtcttgaaacacggaccaaggagtcgtcttcgtatgcgagtgttcgggtgtcaaacccctacgcgtaatgaaagtgaacgtaggagagagcttcggcgcatctccgaccgatcctgatgttctcggatggatttgagtaagagcatacggggccggacccgaaagaaggtgaactatgcctgtatagggtgaagccagaggaaactctggtggaggctcgcagcggttctgacgtgcaaatcgatcgtcaaatatgggcatgggggcgaaagactaatc

SEQ ID NO: 88 - DNA, 290 bp, unidentified
  source 1..290: mol_type = other DNA; note = Acremonium alternatum
atgtgcaaggccggtttcgccggtgacgatgctccccgagctgttttccgtaagtcaaccccactttcgcttcccaagctcctaatcgcccacacctggcgatatgggctttgggggcctgtaagcagccgacacaagactaacgcgatgcgccagcttccattgtcggtcgcccccgtcaccatgggtaagtacgcgcgcaaatgacacctgtcagccccctcgacgagcggcacaggctctgaccattcgatagtatcatgattggtatgggacagaaggactcgtac

SEQ ID NO: 89 - DNA, 380 bp, unidentified
  source 1..380: mol_type = other DNA; note = Acremonium alternatum
ccggactggccgaagacgaagttgtcgggacggaagagctgaccgaaaggaccggcgcggacggcatccatggtaccgggctcgagatcgacgaggacagcgcgaggaacgtacttgttgccagaggcctacagagggtcagcttggccacagactgcgggatactccaaattgctcacctcgttgaagtagacgctcatgcgctcgagctggagctcagaggtgccgttgtagacaccgttgctgtcgaggccatgctcgccagagatggtctgccagaaggcagcaccaatctggttaccctgctcggaggttagacatggtaggcgatatcacatatggcggaagtacttacgcactgaccggtctggaggtgaacc

SEQ ID NO: 90 - DNA, 1231 bp, unidentified
  source 1..1231: mol_type = other DNA; note = Acremonium alternatum
gatgatcgtgatcacttcgggaaaaagcgccttgacctggctgggcccctcttagctaaattgttccgcaacattattcgcaggatcaacaacgagctgtcgacctacctcaggcgatgtgtcgagggcggcaggaacttcaacctcgctgtcggcatcaagcctggcacactgtcgaacgggttgaagtactctttggcaacaggcaactggggagaccaaaagaaggcaatgagctcggttgctggagtgtcccaggttctcaaccgctacacatttgcgtcaaccttgtctcatttgaggcgcaccaacacccccattggccgtgatggaaagctggcgaagcctcggcagctacacaacacgcattggggtcttgtgtgtcccgccgagacccccgagggtcaggcttgtggtttggtgaagaacctgtcactgatgtgtcacgtttccgttggcacacctagcgaacctctctacggatacttcatcaaccgtggcatggaagtgctcgaagagtacgagccccagcggttccccaacgccaccaaggtgttcatcaacggtgcctgggtcggtgtgcacacaagcccgaaagatctcgtggatagcatcatgcatctgcggcgctatggtgacctgaaccatgaagcttccgtcatccgcgacattcgggatcgagagttcagggtcgtcacggatgctggtcgtgttatgcgcccagtattcaccgtgcagcaagaagacaagctagacgggcccgagaagggctcgttgtgcatgaccaaggagcatcttgccggtttggatgactggcatctggtcaacgaggagagggaagagatggccacgggctgggagtacctcgtgaagagtgggtgtattgagtacttggacgccgaagaagaagagacggcaatgatttgcatgacaccagaagacttggagtcttaccgcaaggagaagtacctcgatcagaaaccccaggagcacaacgtggaagccgagcccaacaagcgactcaagacgaagaccaacccgacgacacacatgtacacccactgcgagattcatcccagtatgatcctcggtatctgcgccagcatcatccccttcccggatcataaccaggcatgtctctacgccaccagacctcgagattacttactaatattgcatctagtcgccccgtaatacttaccaatctgccatgggcaagcaggccatgggc

SEQ ID NO: 91 - DNA, 790 bp, unidentified
  source 1..790: mol_type = other DNA; note = Acremonium alternatum
gaatgccccggtcattttggtcacatcgagctggcaaagcccgtttaccaccccggcttcatcaagaaagtcaagaagattttggagattgtctgccacaactgcagcaaggtcttggccgatgaagttggtctcacctgatccatgtcttgttccttagatgctaacatggacctctcagagcgaccccgagtttgtcactgcgatccgtacgcgcgacccgaaagtccgcttccagcgagtctgggctgtgtgcaagaagaagcggaagtgtgagaacgaggatcgccaagacaagaaggaagaggagttcgcgcccggcatgaagccgcagacgcacaaccacggcggctgtggaaacgagatgcccgcggttcgtcaagctggtttgcgtctcaacgcgcagttcgagatcaaggaagagggcggagctaagcgcaaggatactcaagttatcctgcccgaccaagctcacacaatcctgcggcggatatcggaacaggacctccgacacatgggcctcaactcagagtatgcccgcccagagtggatggttcttaccgtccttccggtccccccgcctcccgttcgtccaagtatttccatggacggcactggcacgggaatgcggaacgaggatgatttgacttacaagcttggtgatatcatccgagccaacggaaacgtcaagcaggctatccgcgaaggctcgccggcccacattgctcgcgatttcgaagagctgctccagtaccatgtagccacctacatggataatgatattgctgga

SEQ ID NO: 92 - DNA, 593 bp, unidentified
  source 1..593: mol_type = other DNA; note = Acremonium alternatum
gtcctcgcctaatcaggagtcactagacgacatacccgaggacgacatgatgggcgaccttgcgcttggcctttcgagcagcttcaagcaacacgccctccggaactcaaagggcaagaccttctgggataccttctccgagacgagcagtgtcgcaggaccgagaaccacgccacctccgccgggagtgatggctcgacgtccatcgtccggcaggagtgaggatgtgaccatggattcgccgctccagcaaagcagcatgccttggctacaaacacggcacctttccgactcccagcgctcggactcggcacctgcggccaaggagaaggactccccggcccagccacccaccgctgcagagataacgcgccgaatcaacaacaaacgccgccgtgacgatgacttcgacccggtgagcttcaaacgccgcgcagtgagtcccgggctcagcgtccacaactcgccgctcccgcagagcccaatgcagcagagcggtgcgccatggggttccaggccgggaagcaatgggggcgacaaggcgggaagcagtgcacctagcgaatctggtggtagcacgtcagggaatagga

SEQ ID NO: 93 - DNA, 497 bp, unidentified
  misc_feature 1..497: note = Kingdom: Fungi, Phylum: Ascomycota, Class: Sordariomycetes, Order: Hypocreales, Family: Nectriaceae, Genus: Fusarium
  source 1..497: mol_type = other DNA
cccctgtgacataccaattgttgcctcggcggatcagcccgctcccggtaaaacgggacggcccgccagaggacccctaaactctgtttctatatgtaacttctgagtaaaaccataaataaatcaaaactttcaacaacggatctcttggttctggcatcgatgaagaacgcagcaaaatgcgataagtaatgtgaattgcagaattcagtgaatcatcgaatctttgaacgcacattgcgcccgccagtattctggcgggcatgcctgttcgagcgtcatttcaaccctcaagcccccgggtttggtgttggggatcggcgagcctcacggcaagccggccccgaaatacagtggcggtctcgctgcagcttccattgcgtagtagtaaaaccctcgcaactggtacgcggcgcggccaagccgttaaacccccaacttctgaatgttgacctcggatcaggtaggaatacccgctgaacttaagcatatcaata

SEQ ID NO: 94 - DNA, 552 bp, unidentified
  source 1..552: mol_type = other DNA; note = Purpureocillium lavendulum
ctcccaacccactgtgaccttacctcagttgcctcggcgggaacgccccggccgccggcccccgcgccggcgccggacccaggcgcccgccgcagggaccccaaactctcttgcattacgcccagcgggcggaatttcttctctgagttgcacaagcaaaaacaaatgaatcaaaactttcaacaacggatctcttggttctggcatcgatgaagaacgcagcgaaatgcgataagtaatgtgaattgcagaattcagtgaatcatcgaatctttgaacgcacattgcgcccgccagcattctggcgggcatgcctgttcgagcgtcatttcaaccctcgagcccccccgggggcctcggtgttgggggacggcacaccagccgcccccgaaatgcagtggcgaccccgccgcagcctcccctgcgtagtagcacacacctcgcaccggagcgcggaggcggtcacgccgtaaaacgcccaactttcttagagttgacctcggatcaggtaggaatacccgctgaacttaagcatatcaataagcggaggaa

SEQ ID NO: 95 - DNA, 491 bp, unidentified
  source 1..491: mol_type = other DNA; note = Phialemonium inflatum
acatacccgtaccgttgcctcggcgggcggccccagggcggggccgcagcctccccagcggaggcgcccgccgcaggtcgcaaaactataactatatttagtggcatctctgagtaacttccaaacaatcaaaactttcaacaacggatctcttggttctggcatcgatgaagaacgcagcgaaatgcgataagtaatgtgaattgcagaattcagtgaatcatcgaatctttgaacgcacattgcgcccgccagcattctggcgggcatgcctgtccgagcgtcatttcaaccctcaagccctgcttggtgttggggcactacgcgcgagcgtaggccctcaaaatcagtggcggacccgctggaggtccgggcgtagtaacacatctcgcccgaggtccccagcgtgcccctgccgttaaacccccaaatttacagaaggttgacctcggatcaggtaggaatacccgctgaacttaagcatatcaata

SEQ ID NO: 96 - DNA, 519 bp, unidentified
  source 1..519: mol_type = other DNA; note = Chaetomium coarctatum
ttgtgacgttacctaaaccgttgcttcggcgggcggccccggggtttaccccccgggcgcccctgggccccaccgcgggcgcccgccggaggtcaccaaactcttgataatttatggcctctctgagtcttctgtactgaataagtcaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgttcgagcgtcatttcaaccatcaagccccgggcttgtgttggggacctgcggctgccgcaggccctgaaaagcagtggcgggctcgctgtcacaccgagcgtagtagcatatatctcgctctgggcgtgctgcgggttccggccgttaaaccaccttttaacccaaggttgacctcggatcaggtaggaagacccgctgaacttaagcatatcaataagcggaggaa

SEQ ID NO: 97 - DNA, 527 bp, Chaetomium globosum
  source 1..527: mol_type = other DNA
aaactccctaaaccattgtgaacgttacctataccgttgcttcggcgggcggccccggggtttaccccccgggcgcccctgggccccaccgcgggcgcccgccggaggtcaccaaactcttgataatttatggcctctctgagtcttctgtactgaataagtcaaaactttcaacaacggatctcttggttctggcatcgatgaagaacgcagcgaaatgcgataagtaatgtgaattgcagaattcagtgaatcatcgaatctttgaacgcacattgcgcccgccagcattctggcgggcatgcctgttcgagcgtcatttcaaccatcaagcccccgggcttgtgttggggacctgcggctgccgcaggccctgaaaagcagtggcgggctcgctgtcgcaccgagcgtagtagcatacatctcgctctggtcgcgccgcgggttccggccgttaaaccaccttttaacccaaggttgacctcggatcaggtaggaagacccgctgaacttaagcatatcaataa

SEQ ID NO: 98 - DNA, 1453 bp, Chaetomium globosum
  source 1..1453: mol_type = other DNA
tcttggtcatttagaggaagtaaaagtcgtaacaaggtctccgttggtgaaccagcggagggatcattacagagttgcaaaactccctaaaccattgtgaacgttacctataccgttgcttcggcgggcggccccggggtttaccccccgggcgcccctgggccccaccgcgggcgcccgccggaggtcaccaaactcttgataatttatggcctctctgagtcttctgtactgaataagtcaaaactttcaacaacggatctcttggttctggcatcgatgaagaacgcagcgaaatgcgataagtaatgtgaattgcagaattcagtgaatcatcgaatctttgaacgcacattgcgcccgccagcattctggcgggcatgcctgttcgagcgtcatttcaaccatcaagcccccgggcttgtgttggggacctgcggctgccgcaggccctgaaaagcagtggcgggctcgctgtcgcaccgagcgtagtagcatacatctcgctctggtcgcgccgcgggttccggccgttaaaccaccttttaacccaaggttgacctcggatcaggtaggaagacccgctgaacttaagcatatcaataagcggaggaaaagaaaccaacagggattgccctagtaacggcgagtgaagcggcaacagctcaaatttgaaatctggcttcggcccgagttgtaatttgcagaggaagctttaggcgcggcaccttctgagtcccctggaacggggcgccatagagggtgagagccccgtatagttggatgcctagcctgtgtaaagctccttcgacgagtcgagtagtttgggaatgctgctcaaaatgggaggtaaatttcttctaaagctaaataccggccagagaccgatagcgcacaagtagagtgatcgaaagatgaaaagcactttgaaaagagggttaaatagcacgtgaaattgttgaaagggaagcgcttgtgaccagacttgcgccgggcggatcatccggtgttctcaccggtgcactccgcccggctcaggccagcatcggttctcgcggggggataaaggtcctgggaacgtagctcctccgggagtgttatagcccggggcgtaatgccctcgcggggaccgaggttcgcgcatctgcaaggatgctggcgtaatggtcatcagcgacccgtcttgaaacacggaccaaggagtcaaggttttgcgcgagtgtttgggtgtaaaacccgcacgcgtaatgaaagtgaacgtaggtgagagcttcggcgcatcatcgaccgatcctgatgttttcggatggatttgagtaggagcgttaagccttggacccgaaagatggtgaactatgcttggatagggtgaagccagaggaaactctggtggaggctcgcagcggttctgacgtgcaaatcgatcgtcaaatctgagcatgggggcgaaaga

SEQ ID NO: 99 - DNA, 266 bp, Chaetomium globosum
  source 1..266: mol_type = other DNA
atgtgcaaggccggtttcgccggtgatgatgcaccccgcgctgttttccgtaagtctcccagccccggccccggcccggtcggcgataagccgagctccggacgctcgttggcacaaacagacaagctaacagcgccgtttagcgtcgattgtcggtcgtccccgtcaccatgggtaggctttcagttccggtatctctgcgatatggggtcgctggctaacgcgccgctagtattatgatcggtatggggcagaaggactcgtac

SEQ ID NO: 100 - DNA, 923 bp, Chaetomium globosum
  source 1..923: mol_type = other DNA
tcctgagggaaacttcggcggtaaccagctactagatggttcgattagtctttcgcccccatgctcagatttgacgatcgatttgcacgtcagaaccgctgcgagcctccaccagagtttcctctggcttcaccctatccaagcatagttcaccatctttcgggtccaaggcttaacgctcctactcaaatccatccgaaaacatcaggatcggtcgatgatgcgccgaagctctcacctacgttcactttcattacgcgtgcgggttttacacccaaacactcgcgcaaaaccttgactccttggtccgtgtttcaagacgggtcgctgatgaccattacgccagcatccttgcagatgcgcgaacctcggtccccgcgagggcattacgccccgggctataacactcccggaggagctacgttcccaggacctttatccccccgcgagaaccgatgctggcctgagccgggcggagtgcaccggtgagaacaccggatgatccgcccggcgcaagtctggtcacaagcgcttccctttcaacaatttcacgtgctatttaaccctcttttcaaagtgcttttcatctttcgatcactctacttgtgcgctatcggtctctggccggtatttagctttagaagaaatttacctcccattttgagcagcattcccaaactactcgactcgtcgaaggagctttacacaggctaggcatccaactatacggggctctcaccctctatggcgccccgttccaggggactcagaaggtgccgcgcctaaagcttcctctgcaaattacaactcgggccgaagccagatttcaaatttgagctgttgccgcttcactcgccgttactagggcaatccctgttggtttcttttcctccgcttattgatatgcttaagttcagcgggtc

SEQ ID NO: 101 - DNA, 797 bp, Chaetomium globosum
  source 1..797: mol_type = other DNA
gagtgtccaggtcactttggccacattgagctatccagacccgttttccaccccgggttcatcaggcgtgtcaaaaagttgctcgagatggtctgccacaactgcagcaaggtgttggctgatcgtgttagtgcaccttgcctgaccgagtgatgatttgttttggcatgctaactcttcaccaggaggacgagcaatatgctgctgccatgcggattcgggaccccaaagtacgcttcaagcgagtttgggatatttgcaagagtaagaagcgctgcgaaaacgaagtgcgcaaggggaaagatggcgagttcaaacccgacagcgaaaaccaagccgcagagggtggccatggaggatgtggcaacacgcagccagtcattcgccagcaggctctcaccctgtggggcagcgtcgagaccaaggacgaggatggtgtgaagaccaaggagaagaaggtcatcaccccagaaatggccctgaacatcttccgtcgcatgtcggacgacgagatgattgacattggcctcaatatttcccaagctcgtccggaatggatgatcatcacggttcttcctgtcccgcctcctccggtgcgccccagtatttccatggacggaactggaacaggcttgcggaatgaggacgatctgacgtataaactcggcgatatcatccgcgccaatggcaacgtccgccaggctattgccgagggctctcctcagcatatcatcaccgactttgagaacctactccagtaccacgtcgctacgtacatggataatgacatcgccggt

SEQ ID NO: 102 - DNA, 1132 bp, Chaetomium globosum
  source 1..1132: mol_type = other DNA
cttccgtcaatttctttaagtttcagccttgcgaccatactcccccaggagcccaaacattttgatttatcgtaaggtgccgaacgggtcaaaaaataacgccgtccgatccctaatcggcatagtttaggttaagactacgacggtatctgatcgtattcgatcccctaactttcgttcctgattaatgaaaacatccttggcaaatgctttcgcagtagttagtcttcaataaatccaagaatttcacctctgacaattgaatactgatgcccccgactgtccctattaatcattacggcggtcctagaaaccaacaaaatagaaccacacgtcctattctattattccatgctaatgtattcgagcataggccttctttaagcgatctaatttgttcagagtaaaagtcctggttccccggcacacccagtgaagggcatgcggttctccagaaggaaagacccagccgagccagtgcacgcggtgaggcggaccggccggctaggcccaaggttcaactacgagctttttaacctcaacaactttaatatacgctattggagctggaattaccgcggctgctggcaccagacttgccctccaattgttcctcgttaagggatttaaattgtactcattccaattacaagacccgaaagagccctgtatcagtatttattgtcactacctccccgtgtcgggattgggtaatttgcgcgcctgctgccttcctttggatgtagtagccgtttctcaggctccttctccggggtcgagccctaaccctccgttacccgttgtcaccacggctggccaagacccagccgtcgaaagttgatagggcagaaatttgaatgaaccatcgccggcgcaaggccgtgcgattcgagaagttattatgaatcaccagagagccccgaagggcattggtttttaatctaataaatacatcccttccgaagtcgggatttttagcatgtattagctctagaattaccacggttatccatgtagtaaggtactatcaaataaacgataactgatttaatgagccattcgcagtttcgcggtataattgcttatacttagacatgcatggcttaatctttgagacaagcatatgactact

SEQ ID NO: 103 - DNA, 925 bp, Chaetomium globosum
  source 1..925: mol_type = other DNA
cttccgtcaatttctttaagtttcagccttgcgaccatactcccccaggagcccaaacattttgatttatcgtaaggtgccgaacgggtcaaaaaataacgccgtccgatccctaatcggcatagtttaggttaagactacgacggtatctgatcgtattcgatcccctaactttcgttcctgattaatgaaaacatccttggcaaatgctttcgcagtagttagtcttcaataaatccaagaatttcacctctgacaattgaatactgatgcccccgactgtccctattaatcattacggcggtcctagaaaccaacaaaatagaaccacacgtcctattctattattccatgctaatgtattcgagcataggccttctttaagcgatctaatttgttcagagtaaaagtcctggttccccggcacacccagtgaagggcatgcggttctccagaaggaaagacccagccgagccagtgcacgcggtgaggcggaccggccggctaggcccaaggttcaactacgagctttttaacctcaacaactttaatatacgctattggagctggaattaccgcggctgctggcaccagacttgccctccaattgttcctcgttaagggatttaaattgtactcattccaattacaagacccgaaagagccctgtatcagtatttattgtcactacctccccgtgtcgggattgggtaatttgcgcgcctgctgccttcctttggatgtagtagccgtttctcaggctccttctccggggtcgagccctaaccctccgttacccgttgtcaccacggctggccaagacccagccgtcgaaagttgatagggcagaaatttgaatgaaccatcgccggcgcaaggccgtgcgattcgagaagttattatgaatcaccagagagccccgaag

SEQ ID NO: 104 - DNA, 518 bp, Chaetomium globosum
  source 1..518: mol_type = other DNA
gttcacctccagaccggccagtgcgtaagttggaccgaatcgaacattacgaccgaccggccgcgcaggataactgacatggagctctctagggtaaccaaatcggtgccgctttctggtacgtccaagcaaagcaaacactcttggctgatgacaatcgagactgacttcttttcaggcagaccatctctggcgagcacggcctcgacagcaatggcgtgtatgtgggcatgacagttcccaaccgataaatccccgctcaccgcttcgataggtacaacggcacctccgagctccagctcgagcgtatgaacgtgtacttcaacgaggtcagtcgggtcaaataattttacacgaccgagtgatggcgtgctcatagtattatacaggcttccggcaacaagtatgttcctcgcgctgtcctcgtcgacttggagcccggcaccatggatgccgtccgtgccggccccttcggccagctcttccgcccggacaacttcgtcttcggccagtcgggt

SEQ ID NO: 105 - DNA, 568 bp, Chaetomium globosum
  source 1..568: mol_type = other DNA
gaggaggaggaggagaggttggagagggaggcgttgcgtgccgaggcgctttgtgaggtcaggcgggttatggcgctgctggaggatacgctgcttgcggacgggcgggagtgggttttgggcggtggtggtggcggtgatggtggtggcagtgagggtgcgagaaaagggccgacgttggcggatatcgaggccgtgtgggtgcttcactggatgattggcattcctggtgcgctgttcaacgccgggtatgtgagcgccgagcggtttccgcgggtgtatgcgtgggtggcgcggtttcaggcggcggttggggcggcgaaggccggggtggtggtgaagggcatgagcggggaggaggcggcggtagtgttgaaggggcagagagaaggggtaggatattttgagaaggagggggaggtggacgccgcggacccgatcgtcaaggtgtacggattggagaaagggagcagggtcgaggtgtggccgacggactccggggctgggcatcgggatcagggctgcctggtgagcctcgacgccgaggaaatagtctgggagacggacg

SEQ ID NO: 106 - DNA, 520 bp, Chaetomium globosum
  source 1..520: mol_type = other DNA
ttgtgacgttacctataccgttgcttcggcgggcggccccggggtttaccccccgggcgcccctgggccccaccgcgggcgcccgccggaggtcaccaaactcttgataatttatggcctctctgagtcttctgtactgaataagtcaaaactttcaacaacggatctcttggttctggcatcgatgaagaacgcagcgaaatgcgataagtaatgtgaattgcagaattcagtgaatcatcgaatctttgaacgcacattgcgcccgccagcattctggcgggcatgcctgttcgagcgtcatttcaaccatcaagcccccgggcttgtgttggggacctgcggctgccgcaggccctgaaaagcagtggcgggctcgctgtcgcaccgagcgtagtagcatacatctcgctctggtcgcgccgcgggttccggccgttaaaccaccttttaacccaaggttgacctcggatcaggtaggaagacccgctgaacttaagcatatcaataagcggaggaa

SEQ ID NO: 107 - DNA, 523 bp, Chaetomium globosum
  source 1..523: mol_type = other DNA
cattgtgaacgttacctataccgttgcttcggcgggcggccccggggtttaccccccgggcgcccctgggccccaccgcgggcgcccgccggaggtcaccaaactcttgataatttatggcctctctgagtcttctgtactgaataagtcaaaactttcaacaacggatctcttggttctggcatcgatgaagaacgcagcgaaatgcgataagtaatgtgaattgcagaattcagtgaatcatcgaatctttgaacgcacattgcgcccgccagcattctggcgggcatgcctgttcgagcgtcatttcaaccatcaagcccccgggcttgtgttggggacctgcggctgccgcaggccctgaaaagcagtggcgggctcgctgtcgcaccgagcgtagtagcatacatctcgctctggtcgcgccgcgggttccggccgttaaaccaccttttaacccaaggttgacctcggatcaggtaggaagacccgctgaacttaagcatatcaataagcggaggaa

SEQ ID NO: 108 - DNA, 518 bp, unidentified
  source 1..518: mol_type = other DNA; note = Chaetomium piluliferum
cattgtgacgttaccttcaaaccgttgcttcggcgggcggcccgggtccgcccggtgccccctggccccctagcggggcgcccgccggaggaaaacccaactcttgattattatggcctctctgagtcttctgtactgaataagtcaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgttcgagcgtcatttcaaccatcaagccccaggcttgtgttggggacctgcggctgccgcaggccctgaaaaccagtggcgggctcgctgtcacaccgggcgtagtagattttatctcgctctgggcgtgctgcgggttccggccgttaaaaaacccttttaacccaaggttgacctcggatcaggtaggaatacccgctgaacttaagcatatcaataagcggagga

SEQ ID NO: 109 - DNA, 558 bp, unidentified
  misc_feature 1..558: note = Kingdom: Fungi, Phylum: Ascomycota, Class: Sordariomycetes, Order: Sordariales, Family: Chaetomiaceae, Genus: Chaetomium
  source 1..558: mol_type = other DNA
tcgttggtgaccagcggagggatcattacagagttgcaaaactccctaaaccattgtgaacgttacctaaaccgttgcttcggcgggcggccccggggtttaccccccgggcgcccctgggccccaccgcgggcgcccgccggaggtcaccaaactcttgataatttatggcctctctgagtcttctgtactgaataagtcaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgttcgagcgtcatttcaaccatcaagccccgggcttgtgttggggacctgcggctgccgcaggccctgaaaagcagtggcgggctcgctgtcacaccgagcgtagtagcatatatctcgctctgggcgtgctgcgggttccggccgttaaaccaccttttaacccaaggttgacctcggatcaggtaggaagacccgctgaacttaagcatat

SEQ ID NO: 110 - DNA, 507 bp, unidentified
  misc_feature 1..507: note = Kingdom: Fungi
  source 1..507: mol_type = other DNA
ctttgtgaccttcatacctgttgcttcggcggcgcgcctctcggggcgtgcccgccggcattatcagaatctctgttcgaacccgacgatacttctgagtgttctaagcgaactgttaaaactttcaacaacggatctcttggctccagcatcgatgaagaacgcagcgaaacgcgatatgtaatgtgaattgcagaattcagtgaatcatcgaatctttgaacgcacatggcgccttccagtatcctgggaggcatgcctgtccgagcgtcgtttcaaccctcgagcccccgtggcccggcgttggggacctgcccaggcagtccccgaaaaccagtggcggacccgacgggcccttcctttgcgtagtaacatctgcctcgcatcgggagcccccgggctatccggcctctaaacccccctcaagcccgctccggcggcaccaaggttgacctcggatcaggtaggaatacccgctgaacttaagcatatcaataagcggaggaa

SEQ ID NO: 111 - DNA, 475 bp, unidentified
  misc_feature 1..475: note = Kingdom: Fungi, Phylum: Basidiomycota, Class: Tremellomycetes, Order: Tremellales, Family: Incertae sedis, Genus: Cryptococcus
  source 1..475: mol_type = other DNA
gcctttaccggctggtagggtttggacggtctctttagggagccggcctaatctcacacaccgtgaactgtggcttcggccatttacacaaactgttagtaatgaatgtcatatcataacaaacataaaacttttaacaacggatctcttggctctcgcatcgatgaagaacgcagcgaattgcgataagtaatgtgaattgcagaattcagtgaatcatcgaatctttgaacgcaccttgcgccctttggtattccgaagggcatgcctgtttgagtgtcatgaaacctcaccccatttaggtttttgcctgagtggtcggtggattgggtgttgccgatatactggctcgcctgaaaagcataagcgccttggatgtaatacgtttcatccttctgggtggctgataaccccacatatctcatgatctggcctcaaatcaggtagggctacccgctgaacttaagcatatcaa

SEQ ID NO: 112 - DNA, 499 bp, unidentified
  misc_feature 1..499: note = Kingdom: Fungi, Phylum: Basidiomycota, Class: Tremellomycetes, Order: Tremellales, Family: Incertae sedis, Genus: Cryptococcus
  source 1..499: mol_type = other DNA
ccctttgtgaccttcatacctgttgcttcggcggcgcgcctctcggggcgtgcccgccggcattatcagaatctctgttcgaacccgacnatacttctgagtgttctaagcgaactgttaaaactttcaacaacggatctcttggctccagcatcgatgaagaacgcagcgaaacgcgatatgtaatgtgaattgcagaattcagtgaatcatcgaatctttgaacgcacatggcgccttccagtatcctgggaggcatgcctgtccgagcgtcgtttcaaccctcgagcccccgtggcccggcgttggggacctgcccaggcagtccccgaaaaccagtggcggacccgacgggcccttcctttgcgtagtaacatctgcctcgcatcgggagcccccgggctatccggcctctaaacccccctcaagcccgctccggcggcaccaaggttgacctcggatcaggtaggaatacccgctgaacttaagcatatcaata

SEQ ID NO: 113 - DNA, 169 bp, unidentified
  misc_feature 1..169: note = Kingdom: Fungi
  source 1..169: mol_type = other DNA
taaccctttgttgtccgactctgttgcctccggggcgaccctgccttcgggcgggggctccgggtggacacttcaaactcttgcgtaactttgcagtctgagtaaacttaattaataaattaaaacttttaacaacggatctcttggttctggcatcgatgaagaacgc

SEQ ID NO: 114 - DNA, 1443 bp, unidentified
  misc_feature 1..1443: note = Kingdom: Fungi, Phylum: Ascomycota, Class: Dothideomycetes, Order: Capnodiales, Family: Cladosporiaceae, Genus: Cladosporium
  source 1..1443: mol_type = other DNA
tcttggtcatttagaggaagtaaaagtcgtaacaaggtctccgtaggtgaacctgcggagggatcattacaagtgaccccggtctaaccaccgggatgttcataaccctttgttgtccgactctgttgcctccggggcgaccctgccttcgggcgggggctccgggtggacacttcaaactcttgcgtaactttgcagtctgagtaaacttaattaataaattaaaacttttaacaacggatctcttggttctggcatcgatgaagaacgcagcgaaatgcgataagtaatgtgaattgcagaattcagtgaatcatcgaatctttgaacgcacattgcgccccctggtattccggggggcatgcctgttcgagcgtcatttcaccactcaagcctcgcttggtattgggcatcgcggtccgccgcgtgcctcaaatcgaccggctgggtcttctgtcccctaagcgttgtggaaactattcgctaaagggtgttcgggaggctacgccgtaaaacaaccccatttctaaggttgacctcggatcaggtagggatacccgctgaacttaagcatatcaataagcggaggaaaagaaaccaacagggattgctctagtaacggcgagtgaagcagcaatagctcaaatttgaaatctggcgtcttcgacgtccgagttgtaatttgtagaggatgcttctgagtaaccaccgacctaagttccttggaacaggacgtcatagagggtgagaatcccgtatgcggtcggaaaggtgctctatacgtagctccttcgacgagtcgagttgtttgggaatgcagctctaaatgggaggtaaatttcttctaaagctaaatattggccagagaccgatagcgcacaagtagagtgatcgaaagatgaaaagcactttggaaagagagttaaaaagcacgtgaaattgttaaaagggaagggattgcaaccagacttgctcgcggtgttccgccggtcttctgaccggtctactcgccgcgttgcaggccagcatcgtctggtgccgctggataagacttgaggaatgtagctccttcgggagtgttatagcctcttgtgatgcagcgagcgccgggcgaggtccgcgcttcggctaggatgctggcgtaatggtcgtaatccgcccgtcttgaaacacggaccaaggagtctaacatctatgcgagtgttcgggtgtcaaacccctacgcgtaatgaaagtgaacggaggtgagaaccgcaaggtgcatcatcgaccgatcctgatgtcttcggatggatttgagtaagagcatagctgttgggacccgaaagatggtgaactatgcctgaatagggtgaagccagaggaaactctggtggaggctcgcagcggttctgacgtgcaaatcgatcgtcaaatttgggtataggggcgaaagactaatcg

SEQ ID NO: 115 - DNA, 489 bp, unidentified
  misc_feature 1..489: note = Kingdom: Fungi, Phylum: Ascomycota, Class: Dothideomycetes, Order: Capnodiales, Family: Cladosporiaceae, Genus: Cladosporium
  source 1..489: mol_type = other DNA
ccgggatgttcataaccctttgttgtccgactctgttgcctccggggcgaccctgccttcgggcgggggctccgggtggacacttcaaactcttgcgtaactttgcagtctgagtaaacttaattaataaattaaaacttttaacaacggatctcttggttctggcatcgatgaagaacgcagcgaaatgcgataagtaatgtgaattgcagaattcagtgaatcatcgaatctttgaacgcacattgcgccccctggtattccggggggcatgcctgttcgagcgtcatttcaccactcaagcctcgcttggtattgggcaacgcggtccgccgcgtgcctcaaatcgaccggctgggtcttctgtcccctaagcgttgtggaaactattcgctaaagggtgttcgggaggctacgccgtaaaacaaccccatttctaaggttgacctcggatcaggtagggatacccgctgaacttaagcatatcaa